(19) United States
(12) Patent Application Publication
(10) Pub. No.: US 2019/0167771 A1
(43) Pub. Date: Jun. 6, 2019

(54) CONTROL ANGIOGENESIS BY REGULATING PHOSPHORYLATION OF SERYL-tRNA SYNTHETASE (SerRS)

(71) Applicant: The Scripps Research Institute, La Jolla, CA (US)

(72) Inventors: Xiang-Lei Yang, San Diego, CA (US); Yi Shi, Tianjin (CN); Ze Liu, San Dego, CA (US)

(21) Appl. No.: 16/325,509

(22) PCT Filed: Aug. 14, 2017
(86) PCT No.: PCT/US17/46754

§ 371 Date: Feb. 14, 2019

Related U.S. Application Data
(60) Provisional application No. 62/375,592, filed on Aug. 16, 2016.

(51) Int. Cl. A61K 38/53; A61P 35/00

(52) U.S. Cl. CPC A61K 38/53; C12Y 601/01011; A61P 35/00

(57) ABSTRACT

Disclosed herein are methods and compositions for modulating angiogenesis, and reducing tumor progression by regulating phoosprylation, of seryl-tRNA synthase (SerRS). Also disclosed are related compositions and methods for treating diseases such as cancer.

### REFERENCE TO SEQUENCE LISTING

The present application is being filed along with a Sequence Listing in electronic format. The Sequence Listing is provided as a file entitled PCTSEQLISTING.TXT, created Aug. 9, 2017, which is 56 Kb in size. The information in the electronic format of the Sequence Listing is incorporated herein by reference in its entirety

### STATEMENT REGARDING FEDERALLY SPONSORED R&D

This invention was made with government support under R01 GM088278 and NS085092 awarded by National Institutes of Health. The government has certain rights in the invention.

### BACKGROUND

The present disclosure relates to the fields of molecular biology and medicine. Disclosed herein includes compositions and methods for regulating angiogenesis and tumor progression in a subject through regulating phosphorylation of seryl-tRNA synthetase (SerRS), and related compositions and methods for treating diseases such as cancer.

SerRS is a member of the aminoacyl-tRNA synthetase family responsible for charging serine onto its cognate tRNA to generate substrates for protein biosynthesis. Studies have suggested a role of SerRS in vascular development independent of its aminoacylation activity.

### SUMMARY

Disclosed herein is a method of reducing tumor progression in a subject, where the method comprises: administering a composition comprising a mutant seryl-tRNA synthetase (SerRS) protein to a subject in need, wherein the mutant SerRS protein is a phosphorylation-deficient mutant SerRS protein, whereby tumor progression is reduced in the subject.

In some embodiments, the composition is a pharmaceutical composition. In some embodiments, the mutant SerRS protein has a decreased level of phosphorylation by ataxia telangiectasia mutated kinase (ATM), ataxia telangiectasia and Rad3-related kinase (ATR), or both. In some embodiments, the maximum level of phosphorylation of the mutant SerRS protein is less than 50% of that of the corresponding wildtype SerRS protein. In some embodiments, the maximum level of phosphorylation of the mutant SerRS protein is less than 10% of that of the corresponding wildtype SerRS protein.

In some embodiments, the mutant SerRS protein comprises an amino acid substitution at one or more of residues T22, X79, S86, X101, X142, S217, 5241, S255, S258, S262, S368, S394, S396, T214, T501, X220, Y248, and Y263 relative to the corresponding wildtype SerRS protein, wherein X is serine, tyrosine, or threonine. In some embodiments, the mutant SerRS protein comprises an amino acid substitution at residue S101, S241, or both relative to the corresponding wildtype SerRS protein. In some embodiments, the mutant SerRS protein comprises an amino acid substitution X101A, S241A, or both relative to the corresponding wildtype SerRS protein, wherein X is serine or threonine. In some embodiments, the mutant SerRS protein comprises an amino acid deletion at one or more of residues T122, X79, S86, X101, X142, S217, S241, S255, S258, S262, S368, S394, S396, T214, T501, X220, Y248, and Y263 relative to the corresponding wildtype SerRS protein, wherein X is serine, tyrosine or threonine. In some embodiments, the mutant SerRS protein comprises an amino acid deletion at residue X101, S241, or both, wherein X is serine or threonine.

In some embodiments, the mutant SerRS protein is a vertebrate SerRS protein. In some embodiments, the mutant SerRS protein is a human SerRS protein. In some embodiments, the mutant SerRS protein comprises an amino acid sequence having at least 90% identity to the amino acid sequence set forth in SEQ ID NO: 1, SEQ ID NO: 42, SEQ ID NO: 44, or SEQ ID NO: 46, and comprise an amino acid deletion at one or both of residues X101 and S241 of SEQ ID NO: 1, SEQ ID NO: 42, SEQ ID NO: 44, or SEQ JD NO: 46, wherein X is serine or threonine. In some embodiments, the mutant SerRS protein comprises an amino acid sequence having at least 90% identity to the amino acid sequence set forth in SEQ ID NO: 1 and comprises an amino acid substitution at one or both of residues S101 and S241 of SEQ ID NO: 1, wherein the amino acid substitution is selected from the group consisting of serine-to-alanine, serine-to-glycine, serine-to-lysine, serine-to-arginine, serine-to-asparagine, serine-to-glutamine, serine-to-histidine, serine-to-cysteine, serine-to-valine, serine-to-leucine, serine-to-isoleucine, serine-to-proline, serine-to-methionine, serine-to-tryptophan, and serine-to-phenylalanine. In some embodiments, the mutant SerRS protein comprises an amino acid sequence having at least 90% identity to the amino acid sequence set forth in SEQ ID NO: 1 and comprises an amino acid substitution at one or both of residues S101 and S241 of SEQ ID NO: 1, wherein the amino acid substitution is serine-to-alanine or serine-to-glycine. In some embodiments, the mutant SerRS protein comprises an amino acid sequence set forth in SEQ ID NO: 2, SEQ ID NO: 3, or SEQ ID NO: 4.

In some embodiments, the reduction of tumor progression is achieved by reducing angiogenesis in the subject. In some embodiments, the angiogenesis is hypoxia-induced angiogenesis. In some embodiments, the tumor progression is metastasis. In some embodiments, the tumor is solid tumor. In some embodiments, the solid tumor is sarcomas, carcinomas, lymphomas, or a combination thereof. In some embodiments, the tumor is hematological malignancy. In some embodiments, the tumor is cervical cancer, colon cancer, liver cancer, prostate cancer, melanoma, ovarian cancer, lung cancer, renal cell carcinoma, Schwannoma, mesothelioma, acute myeloid leukemia, multiple myeloma, non-Hodgkin lymphoma, or a combination thereof. In some embodiments, the phosphorylation-deficient mutant SerRS protein represses transcription of vascular endothelial growth factor (VEGF) in the subject. In some embodiments, the VEGF is VEGFA. In some embodiments, the tumor progression in the subject is reduced by at least 50% as compared to subjects received no treatment.

Also disclosed herein is a mutant seryl-tRNA synthetase (SerRS) protein, where the mutant SerRS protein is phosphorylation-deficient. In some embodiments, the mutant SerRS protein comprises an amino acid substitution at one or more of residues T22, X79, S86, X101, X142, S217, S241, S255, S258, S262, S368, S394, S396, T214, T501, X220, Y248, and Y263 relative to the corresponding wild type SerRS protein, wherein X is serine, tyrosine or threonine. In some embodiments, the mutant SerRS protein comprises a amino acid substitution at X101, S241, or both relative to the corresponding wildtype SerRS protein, wherein X is serine or threonine. In some embodiments, the mutant SerRS protein comprises an amino acid substitution X101A, S241A, or both relative to the corresponding wildtype SerRS protein, wherein X is serine or threonine. In some embodiments, the mutant SerRS protein comprises an amino acid deletion at one or more of residues T22, X79, S86, X101, X142, S217, S241, S255, S258, S262, S368, S394, S396, T214, T501, X220, Y248, and Y263 relative to the corresponding wildtype SerRS protein, wherein X is serine, tyrosine, or threonine. In some embodiments, the mutant SerRS comprises an amino acid deletion at Serine 101, Serine 241, or both relative to the corresponding wildtype SerRS protein.

In some embodiments, the mutant SerRS protein is a vertebrate protein. In some embodiments, the mutant SerRS protein is a human protein.

In some embodiments, the mutant SerRS protein comprises an amino acid sequence having at least 90% identity to the amino acid sequence set forth in SEQ ID NO:1, SEQ ID NO: 42, SEQ ID NO: 44, or SEQ ID NO: 46, and comprises an amino acid deletion at one or both of residues X101 and S241, wherein X is serine or threonine.

In some embodiments, the mutant SerRS protein comprises an amino acid sequence having at least 90% identity to the amino acid sequence set forth in SEQ ID NO: 1 and comprises an amino acid substitution at one or both of residue S101 and S241 in SEQ ID NO: 1, wherein the amino acid substitution is selected from serine-to-alanine, serine-to-glycine, serine-to-lysine, serine-to-arginine, serine-to-asparagine, serine-to-glutamine, serine-to-histidine, serine-to-cysteine, serine-to-valine, serine-to-leucine, serine-to-isoleucine, serine-to-proline, serine-to-methionine, serine-to-tryptophan, and serine-to-phenylalanine.

In some embodiments, the mutant SerRS protein comprises an amino acid sequence having at least 90% identity to the amino acid sequence set forth in SEQ ID NO: 1 and comprises an amino acid substitution at one or both of residue S101 and S241 in SEQ ID NO: 1, wherein the amino acid substitution is serine-to-alanine or serine-to-glycine. In some embodiments, the mutant SerRS protein comprises an amino acid sequence set forth in SEQ ID NO: 2, SEQ ID NO: 3, or SEQ ID NO: 4.

Also disclosed herein is a mutant seryl-tRNA synthetase (SerRS) protein, where the mutant SerRS protein is deficiency in repressing VEGF transcription as compared to the corresponding wildtype SerRS protein, or is effective in stimulating VEGF transcription.

In some embodiments, the mutant SerRS protein comprises an amino acid substitution at one or more of residues T22, X79, S86, X101, X142, S217, S241, 5255, S258, S262, S368, S394, S396, T214, T501, X220, Y248, and Y263 relative to the corresponding wild type SerRS protein, wherein X is serine, tyrosine or threonine. In some embodiments, the mutant SerRS protein comprises an amino acid substitution at residue X101, S241, or both relative to the corresponding wildtype SerRS protein, wherein X is serine or threonine. In some embodiments, the mutant SerRS protein comprises an amino acid substitution X101D, S241D, or both relative to the corresponding wildtype SerRS protein, wherein X is serine or threonine.

In some embodiments, the mutant SerRS protein is a vertebrate protein. In some embodiments, the mutant SerRS protein is a human protein.

In some embodiments, the mutant SerRS protein comprises an amino acid sequence having at least 90% identity to the amino acid sequence set forth in SEQ ID NO: 1, SEQ ID NO: 42, SEQ ID NO: 44, or SEQ ID NO: 46, and comprises an amino acid substitution at one or both of amino acid residues X101 and S241 in SEQ ID NO: 1, SEQ ID NO: 42, SEQ ID NO: 44, SEQ ID NO: 46, wherein X is serine or threonine and wherein the amino acid substitution is serine-to-aspartic acid, serine-to-glutamic acid, threonine-to-aspartic acid or threonine-to-glutamic acid. In some embodiments, the mutant SerRS protein comprises an amino acid sequence of SEQ ID NO: 5 or SEQ ID NO: 6.

In some embodiments, the mutant SerRS protein does not repress VEGF transcription. In some embodiments, the mutant SerRS protein stimulates VEGF transcription.

Also disclosed herein is a pharmaceutical composition. In some embodiments, the pharmaceutical composition comprises one or more of the mutant SerRS proteins disclosed herein; and a pharmaceutically acceptable excipient.

Also disclosed herein is a method of promoting angiogenesis in a subject. In some embodiments, the method comprises: administering a composition comprising a mutant seryl-tRNA synthetase (SerRS) protein to a subject in need, wherein the mutant SerRS protein is deficiency in repressing VEGF transcription as compared to the corresponding wildtype SerRS protein or is effective in stimulating VEGF transcription, whereby angiogenesis is promoted in the subject. In some embodiments, the composition is a pharmaceutical composition. In some embodiments, the subject suffers from one or more of ischemic heart disease, cardiovascular diseases, and neurological diseases.

In some embodiments, the repression of VEGF transcription by the mutant SerRS protein is less than 50% of the repression of VEGF transcription by the corresponding wildtype SerRS protein. In some embodiments, the mutant SerRS protein does not repress VEGF transcription. In some embodiments, the mutant SerRS stimulates VEGF transcription.

In some embodiments, the mutant SerRS protein comprises an amino acid substitution at one or more of residues T22, X79, 886, X101, X142, S217, S241, S255, S258, S262, S368, S394, S396, T214, T501, X220, Y248, and Y263 relative to the corresponding wild type SerRS protein, wherein X is serine, tyrosine or threonine.

In some embodiments, the mutant SerRS protein comprises an amino acid substitution at X101, S241, or both relative to the corresponding wildtype SerRS protein, wherein X is serine or threonine. In some embodiments, the mutant SerRS protein comprises an amino acid substitution X101D, S241D, or both relative to the corresponding wildtype SerRS protein, wherein X is serine or threonine. In some embodiments, the mutant SerRS protein is a vertebrate protein. In some embodiments, the mutant SerRS protein is a human protein.

In some embodiments, the mutant SerRS protein comprises an amino acid sequence having at least 90% identity to the amino acid sequence set forth in SEQ ID NO: 1, SEQ ID NO: 42, SEQ ID NO: 44, or SEQ ID NO: 46, and comprises an amino acid substitution on one or both of residues X101 and S241 in SEQ ID NO: 1, SEQ ID NO: 42, SEQ ID NO: 44, or SEQ ID NO: 46, wherein X is serine or threonine and wherein the amino acid substitution is serine-to-aspartic acid, serine-to-glutamic acid, threonine-to-aspartic acid or threonine-to-glutamic acid. In some embodiments, the mutant SerRS protein comprises an amino acid sequence of SEQ ID NO: 5 or SEQ ID NO: 6.

Also disclosed herein is a method of reducing angiogenesis in a subject. In some embodiments, the method comprises: administering a composition comprising a seryl-tRNA synthetase (SerRS) phosphorylation inhibitor to a subject in need, whereby angiogenesis is reduced in the subject. In some embodiments, the composition is a pharmaceutical composition. In some embodiments, the SerRS phosphorylation inhibitor is an inhibitor for ataxia telangiectasia mutated kinase (ATM), ataxia telangiectasia and Rad3-related kinase (ATR), or both. In some embodiments, the SerRS phosphorylation inhibitor is an ATM inhibitor. In some embodiments, the SerRS phosphorylation inhibitor is an ATR inhibitor.

### BRIEF DESCRIPTION OF THE DRAWINGS

FIGS. 1A-G show SerRS is important for hypoxia induced VEGFA expression and is phosphorylated by ATM and ATR kinases under hypoxia at serine 101 and serine 241 residues. In FIG. 1A, HEK293 cells were transfected with shRNAs targeting SerRS (sh-SerRS) or GlyRS (sh-GlyRS), or nonspecific control shRNA (sh-Control). 48 hours post-transfection, cells were cultured under hypoxia or normoxia conditions for 12 hours. Immunoblot of cell lysates with anti-SerRS, anti-GlyRS and anti-β-actin antibodies (left). VEGFA expression levels were measured by qRT-PCR (right) and relative induction of VEGFA transcription under hypoxia were plotted (right, inset) (means±SEM. from four independent experiments; *P<0.01, **P<0.0001). FIG. 1B shows alignment of sequences flanking serine 101 and serine 241 (shaded) of human SerRS with the same regions of other invertebrate and vertebrate SerRS sequences. The conserved ATM/ATR substrate motif residues are underlined. In FIG. 1C, recombinant his6-tagged human SerRS or GlyRS were incubated with γ-³²P-ATP and HEK293 cell nuclear extracts which are treated with or without double-stranded DNA oligoes mimicking DNA damage stimuli to activate ATM/ATR/DNA-PK kinases. Recombinant proteins were then purified by Ni-NTA beads and subjected to SDS-PAGE and autoradiography. In FIG. 1D, recombinant his6-tagged two human aminoacyl tRNA synthetases (AARS): TyrRS and GlyRS, wild type SerRS, or SerRS mutants with single or double substitutions of serine 101 and serine 241 with alanine (S101A, S241A, and S101A/S241A) were treated as described in FIG. 1C and phosphorylated recombinant proteins were immunoblotted with antibodies as indicated after purification by Ni-NTA beads. In FIG. 1E, HEK293 cells were cultured under hypoxia for 3, 6, and 12 hours. Phosphorylated SerRS was immunoblotted with specific anti-p-SQ antibody after SerRS proteins were immunoprecipitated (IP). Cell lysates were immunoblotted with indicated antibodies for known ATM and ATR substrates (Chk1 and P53). In FIG. 1F, Flag-tagged wild type and mutant SerRS constructs were transfected into HEK293 cells. 24 hours post-transfection, cells were treated with hypoxia stress for 12 hours and ectopically expressed SerRS was then purified by immunoprecipitation (IP) using anti-Flag antibody and immunoblot (IB) with anti-P-SQ antibody and anti-Flag antibody. In FIG. 1G, HEK293 cells were transfected with siRNA against ATM (si-ATM) or ATR (si-ATR) and treated with hypoxia stress for 12 hours. Phosphorylated SerRS were immunoblotted as described in FIG. 1E. Cell lysates were immunoblotted with anti-ATM and anti-ATR antibodies.

FIGS. 2A-G show that SerRS phosphorylation at serine 101 and serine 241 inhibits its function in repressing VEGFA expressing and vascular development. In FIG. 2A, wild type SerRS (SerRS^{WT}), its mutants with double substitutions of serine 101 and 241 with alanine or aspartic acid residues (SerRS^{S101A/S241A} and SerRS^{S101D/S241D}), or empty vector were transfected into HEK293 cells. The expression levels of SerRS proteins was measured by immunoblot (bottom) and VEGFA expression was determined by qRT-PCR (means±SEM from three independent experiments; ***P<0.0001). In FIGS. 2B-D, the roles of SerRS^{S101A/S241A} and SerRS^{S101D/S241D} in regulating vascular development in vivo in zebrafish were examined by co-injection of SerRS^{S101A/S241A} and SerRS^{S101D/S241D} mRNAs into one-cell-stage embryos whose endogenous SerRS was knocked down by injection of antisense morpholino (SerRS-MO) that led to abnormal high expression of Vegfa and hypervascularization (shown by short bold arrows in FIG. 2C). At 3 day post fertilization (dpf), embryos were harvested and Vegfa expression levels were measured by qRT-PCR (means±SEM, n=125-211; *P<0.01, **P<0.001) (B). The development of intersegmental vessel (ISV) at 3 dpf was examined (C) and the statistics of ISV abnormal branching rescued by SerRS^{WT} or mutant SerRS mRNA injections was analyzed (D; χ²-test,* P>0.05 vs SerRS^{WT}, **P>0.1 vs SerRS-MO, ***P<1×10⁻²⁸ vs control-MO). In FIG. 2E, the binding affinities of SerRS^{WT} or SerRS^{S101D/S241D} with ³²P labeled DNA fragments corresponding to SerRS binding site on human VEGFA promoter were examined by EMSA. In FIG. 2F, the binding of SerRS^{WT}, SerRS^{S101A/S241A}, or SerRS^{S101D/S241D} on VEGFA promoter in HEK293 cells was examined by chromatin immunoprecipitation (ChIP) and qPCR (means±SEM from two independent experiments; ***P<0.0001). In FIG. 2G, the binding of endogenous SerRS on VEGFA promoter during hypoxia course was monitored by ChIP (means±SEM from three independent experiments; *P<0.05, **P<0.005 vs normal).

FIGS. 3A-C show that SerRS phosphorylation by ATM/ATR is an important pathway that regulates VEGFA induction under hypoxia. In FIG. 3A, HEK293 cells were pretreated with specific ATM inhibitor KU-55933 (5 μM) or specific ATR inhibitor VE-821 (5 μM) before stressed under hypoxia for another 12 hours. VEGFA mRNA levels were then measured by qRT-PCR (means±SEM from two independent experiments; *P<0.05, **P<0.0001). In FIG. 3B, HEK293 cells were transiently transfected with SerRS^{WT} or SerRS^{S101A/S241A} constructs or empty control vector. 24 hours post-transfection, VEGFA mRNA induction by 12-hour-hypoxia treatment was monitored by qRT-PCR (means±SEM from four independent experiments; *P<0.05, **P<0.01, ***P<0.005). In FIG. 3C, HEK293 cells were transiently transfected with indicated constructs. 36 hours post-transfection, VEGFA mRNA induction by 12-hour-hypoxia treatment was monitored by qRT-PCR (means±SEM from four independent experiments; *P<0.0, **P<0.001, ***P<0.0001). The protein levels of SerRS, HIF-1α, and β-actin were examined by western blot (bottom).

FIGS. 4A-C show that SerRS inactivation by phosphorylation at serine 101 and serine 241 is important for angiogenesis under hypoxia. In FIG. 4A, mouse 3B11 endothelial cells were stably transfected with mouse wild type SerRS or mutant SerRS and the expression of SerRS were examined by immunoblot with anti-SerRS antibody and quantified by the density of the bands. In FIGS. 4B-C, Matrigel plug angiogenesis assay was performed with stably transfected 3B11 cells in C3H/HeJ mice. Matrigel plugs (dash lines enclosed regions) excised 14 days after implantation were analyzed by immunohistochemistry for CD31 (FIG. 4B) and microvessel density was quantified (FIG. 4C) (n=10-15).

FIGS. 5A-F show that Phosphorylation-deficient SerRS can strongly suppress tumor angiogenesis and tumor growth. In FIG. 5A, human breast cancer cells MDA-MB-231 were stably transfected with human wild type (SerRS^{WT}) and mutant SerRS (SerRS^{AA}, SerRS^{DD}). The expression of SerRS was monitored by immunoblot. In FIGS. 5B-C, the engineered MDA-MB-231 cells (10⁶ per mouse) were implanted into mouse fat pad in mammal glands to form tumor xenografts. 14 days later, tumor xenografts were excised and subjected to immunohistochemistry for CD31 (FIG. 5B) and subsequent quantification of blood vessels (FIG. 5C) (n=5-6). In FIGS. 5D-F, MDA-MB-231 cells (10⁶ per mouse) stably transfected with SerRS^{WT}, SerRS^{AA}, HIF1-specific shRNA (HIF^{KD}), both SerRS^{AA} and HIF^{KD} or empty vector were implanted into mouse fat pad in mammal glands. The sizes of the tumor xenografts were measured until the mice were sacrificed on 35 days (FIG. 5D) and the tumor angiogenesis was measured by immunohistochemistry for CD31 (FIG. 5E) and VEGFA (FIG. 5F) (n=4-10). Scale bars represent 100 μm.

FIG. 6 shows an illustrative schematic diagram of ATM/ATR-SerRS pathway in hypoxia induced angiogenesis.

FIGS. 7A-D show that SerRS is phosphorylated by ATM/ATR kinases under hypoxia and UV irradiation, related to FIGS. 1A-G. FIG. 7A is an immunoblot shown that SerRS protein levels were not changed in HEK293 under hypoxia stress for 12 hours. In FIG. 7B, recombinant wild type and mutant SerRS proteins were incubated with HEK293 nuclear extracts in buffers containing γ-³²P-ATP and double-stranded DNA oligoes that activated ATM/ATR/DNA-PK kinases. His6-tagged SerRS proteins were then purified by Ni-NTA and subjected to SDS-PAGE and autoradiography. In FIG. 7C, HEK293 cells were pretreated with specific ATM inhibitor KU-55933 and ATR inhibitor VE-821 for one hour and then cultured under hypoxia condition for 12 hours. Cell lysates were subjected to IP with anti-SerRS antibody followed by immunoblot (IB) with anti-P-SQ antibody for detecting phosphorylated SerRS (P-SerRS). Phosphorylation of known ATM/ATR substrates (Chk1 and Chk2) was also immunoblotted. In FIG. 7D, HEK293 cells were exposed to 50 J/cm² UV light and cell lysates were subjected to IP and IB as described in FIG. 7C.

FIGS. 8A-D show that SerRS phosphorylation at serine 101 and serine 241 does not affect its nuclear localization and its interaction with SIRT2, related to FIGS. 2A-G. In FIG. 8A, HEK293 cells were cultured under hypoxia for 12 hours and subjected to cell fractionation. The cytosolic fractions (Cy), nuclear fractions (Nu), and the whole cell lysates (WCL) were examined by immunoblot with antibodies against SerRS, nuclear protein Lamin A/C, and cytosolic protein α-tubulin. In FIG. 8B, HEK293 cells were transfected with Flag-tagged SerRS^{WT}, SerRS^{S101A/S241A}, or SerRS^{S101D/S241D} and subjected to cell fractionation and IB with anti-Flag, anti-Lamin A/C, and anti-α-tubulin antibodies. In FIG. 8C, HEK293 cells were cultured under hypoxia for 6 hours and 12 hours. Cells were then lysed and subjected to IP with anti-SerRS antibody and IB with both anti-SerRS and anti-SIRT2 antibodies. In FIG. 8D, HEK293 cells were co-transfected with V5-tagged SIRT2 and Flag-tagged wild type or mutant SerRS. 24 hours post-transfection, cell lysates were subjected to IP with anti-Flag antibody and IB with anti-V5 antibody.

FIG. 9 shows images from an Matrigel plug angiogenesis assay with mouse 3B11 cells, related to FIGS. 4A-F. The hypoxic environment in the matrigel plugs (encircled in dash lines) were examined by immunohistochemistry using anti-HIF-1α antibody.

FIG. 10 shows how modification on potential phosphorylation sites on SerRS affected VEGFA expression.

FIG. 11 shows a sequence alignment for human, mouse, zebrafish, and frog SerRS proteins. Various phosphorylation sites on human SerRS (for example, T22, S79, S86, S101, S142, T214, S217, Y220, Y248, S255, S258, S262, Y263, T501, and S241) and their corresponding amino acid residues in mouse, zebrafish and frog SerRS proteins are show in bold and highlighted.

FIG. 12 shows the binding of endogenous SerRS, c-Myc, and Hif1α on VEGFA promoter in HEK293 cells during hypoxia course monitored by chromatin IP (ChIP) (means±SEM from three independent experiments; **P<0.005 vs 0 h).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

### General Techniques

The practice of the techniques described herein may employ, unless otherwise indicated, conventional techniques and descriptions of organic chemistry, polymer technology, molecular biology (including recombinant techniques), cell biology, biochemistry, sequencing technology, and micro- and nano-fabrication which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, Gabriel, Stephens, Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Stryer, Biochemistry (4th Ed.) (1995) W.H. Freeman, New York N.Y.; Gait, Oligonucleotide Synthesis: A Practical Approach (2002) IRL Press, London; Nelson and Cox, Lehninger, Principles of Biochemistry (2000) 3rd Ed., W. H. Freeman Pub., New York, N.Y.; Berg, et al., Biochemistry (2002) 5th Ed., W.H. Freeman Pub., New York, N.Y., Jaeger, Introduction to Microelectronic Fabrication (2002) 2nd Ed., Prentice Hall, and Madou, Fundamentals of Microfabrication (2002) all of which are herein incorporated in their entireties by reference for all purposes.

### Some Definitions

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, N.Y. 1994). All publications mentioned herein are incorporated by reference for the purpose of describing and disclosing devices, formulations and methodologies that may be used in connection with the presently described methods and disclosures.

For purposes of the present disclosure, the following terms are defined below.

The terms “polypeptide”. “oligopeptide”, “peptide,” and “protein” are used interchangeably herein to refer to polymers of amino acids of any length, e.g., at least 5, 6, 7, 8, 9, 10, 20, 30, 40, 50, 100, 200, 300, 400, 500, 1,000 or more amino acids. The polymer may be linear or branched, it may include, for example, modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The terms “polynucleotide,” “oligonucleotide,” “nucleic acid” and “nucleic acid molecule” are used interchangeably herein to refer to a polymeric form of nucleotides of any length, e.g., at least 8, 9, 10, 20, 30, 40, 50, 100, 200, 300, 400, 500, 1,000 or more nucleotides, and may include ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. Thus, these terms include, for example, 3′-deoxy-2′,5′-DNA, oligodeoxyribonucleotide N3′ to P5′ phosphoramidates, 2′-O-alkyl-substituted RNA, hybrids between DNA and RNA or between PNAs and DNA or RNA, and also include known types of modifications, for example, labels, alkylation, “caps,” substitution of one or more of the nucleotides with an analog, intemucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g., nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

As used herein, “sequence identity” or “identity” or “homology” in the context of two protein sequences or two nucleotide sequences includes reference to the amino acid residues or the nucleotides in the two sequences which are the same when aligned for maximum correspondence over a specified comparison window. The portion of the amino acid sequence or nucleotide sequence in the comparison window may comprise additions or deletions (i.e., gaps) as compared to the reference sequence for optimal alignment of the two sequences. When percentage of sequence identity is used in reference to proteins it is recognized that residue positions which are not identical often differ by conservative amino acid substitutions, where amino acids are substituted for other amino acid residues with similar chemical properties (e.g. charge or hydrophobicity) and therefore do not change the functional properties of the molecule. Where sequences differ in conservative substitutions, the percentage sequence identity may be adjusted upwards to correct for the conservative nature of the substitutions. Sequences, which differ by such conservative substitutions are said to have “sequence similarity” or “similarity”. Means for making these adjustments are well known to persons of skill in the art. The percentage is calculated by determining the number of positions at which the identical amino acid or nucleic acid base residue occurs in both sequences to yield the number of matched positions, dividing the number of matched positions by the total number of positions in the window of comparison and multiplying the result by 100 to yield the percentage of sequence identity. Typically this involves scoring a conservative substitution as a partial rather than a full mismatch, thereby increasing the percentage sequence identity. Thus, for example, where an identical amino acid is given a score of 1 and a non-conservative substitution is give a score of zero, a conservative substitution is given a score between 0 and 1. The scoring of conservative substitutions is calculated, e.g. according to the algorithm of Meyers and Miller (Computer Applic. Biol. Sci., 1998, 4, 11-17).

As used herein, the term “homologue” is used to refer to a nucleic acid or protein which differs from a naturally occurring nucleic acid (i.e., the “prototype” or “wild-type” nucleic acid) or protein by minor modifications to the naturally occurring nucleic acid or amino acid, but which maintains the basic nucleotide or protein structure of the naturally occurring form. Such changes include, but are not limited to: changes in one or a few nucleotides, including deletions (e.g., a truncated version of the nucleic acid) insertions and/or substitutions. A homologue can have enhanced, decreased, or substantially similar properties as compared to the naturally occurring nucleic acid. A homologue can be complementary or matched to the naturally occurring nucleic acid. Homologues can be produced using techniques known in the art for the production of nucleic acids including, but not limited to, recombinant DNA techniques, chemical synthesis, or any combination thereof.

As used herein, “complementary or matched” means that two nucleic acid sequences have at least 50% sequence identity. For example, the two nucleic acid sequences can have at least 60%, 70%, 80%, 90%, 95%, 96%, 97%, 98%, 99% or 100% of sequence identity. “Complementary or matched” also means that two nucleic acid sequences can hybridize under low, middle and/or high stringency condition(s).

As used herein, “substantially complementary or substantially matched” means that two nucleic acid sequences have at least 90% sequence identity. For example, the two nucleic acid sequences can have at least 95%, 96%, 97%, 98%, 99% or 100% of sequence identity. Alternatively, “substantially complementary or substantially matched” means that two nucleic acid sequences can hybridize under high stringency condition(s).

As used herein, the term “subject” is an animal, such as a vertebrate (for example zebrafish), preferably a mammal. The term “mammal” is defined as an individual belonging to the class Mammalia and includes, without limitation, humans, domestic and farm animals, and zoo, sports, or pet animals, such as sheep, dogs, horses, cats or cows. In some embodiments, the subject is mouse or rat. In some embodiments, the subject is human.

As used herein, the term “treatment” refers to an intervention made in response to a disease, disorder or physiological condition manifested by a patient, particularly a patient suffering from one or more angiogenesis-related diseases, and/or cancer. The aim of treatment may include, but is not limited to, one or more of the alleviation or prevention of symptoms, slowing or stopping the progression or worsening of a disease, disorder, or condition and the remission of the disease, disorder or condition. In some embodiments, “treatment” refers to therapeutic treatment and/or prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing disease symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

The pharmaceutically acceptable or appropriate carrier may include other compounds known to be beneficial to an impaired situation of the GI tract, (e.g., antioxidants, such as Vitamin C, Vitamin E, Selenium or Zinc); or a food composition. The food composition can be, but is not limited to, milk, yoghurt, curd, cheese, fermented milks, milk based fermented products, ice-creams, fermented cereal based products, milk based powders, infant formulae, tablets, liquid bacterial suspensions, dried oral supplement, or wet oral supplement.

A therapeutic agent or a protective agent may comprise a “drug.” As used herein, a “drug” refers to a therapeutic agent or a diagnostic agent and includes any substance, other than food, used in the prevention, diagnosis, alleviation, treatment, or cure of a disease. Stedman's Medical Dictionary, 25th Edition (1990). The drug can include any substance disclosed in at least one of: The Merck Index, 12th Edition (1996); Pei-Show Juo, Concise Dictionary of Biomedicine and Molecular Biology, (1996); U.S. Pharmacopeia Dictionary, 2000 Edition; and Physician's Desk Reference, 2001 Edition. In some embodiments, the therapeutic agent is one of the embodiments of the compositions described herein.

In some embodiments, the drug used in the therapeutic system will often be placed on, embedded, encapsulated or otherwise incorporated into a delivery matrix. The delivery matrix may be included in or on either the first skeletal structure or the second cushioning structure, or both. The delivery matrix, in turn, comprises either a biodegradable or a non-biodegradable material. The delivery matrix may include, although it is not limited to, a polymer. Examples of biodegradable polymers include protein, hydrogel, polyglycolic acid (PGA), polylactic acid (PLA), poly(L-lactic acid) (PLLA), poly(L-glycolic acid) (PLGA), polyglycolide, poly-L-lactide, poly-D-lactide, poly(amino acids), polydioxanone, polycaprolactone, polygluconate, polylactic acid-polyethylene oxide copolymers, modified cellulose, collagen, polyorthoesters, polyhydroxybutyrate, polyanhydride, polyphosphoester, poly(alpha-hydroxy acid), and combinations thereof. Non-biodegradable polymers may comprise silicone, acrylates, polyethylenes, polyurethane, polyurethane, hydrogel, polyester (e.g., DACRON® from E. I. Du Pont de Nemours and Company, Wilmington, Del.), polypropylene, polytetrafluoroethylene (PTFE), expanded PTFE (ePTFE), polyether ether ketone (PEEK), nylon, extruded collagen, polymer foam, silicone rubber, polyethylene terephthalate, ultra-high molecular weight polyethylene, polycarbonate urethane, polyurethane, polyimides, stainless steel, nickel-titanium alloy (e.g., Nitinol), titanium, stainless steel, cobalt-chrome alloy (e.g., ELGILOY® from Elgin Specialty Metals, Elgin, Ill.; CONICHROME® from Carpenter Metals Corp., Wyomissing, Pa.). In one embodiment, the hydrogel may comprise poly(alkyleneoxides), such as poly(ethyleneoxide), also known as polyethyleneglycols or PEGs.

The term “comprising” as used herein is synonymous with “including,” “containing,” or “characterized by,” and is inclusive or open-ended and does not exclude additional, unrecited elements or method steps.

A tumor, also known as a neoplasm, is generally refers to an abnormal mass of tissue which may be, for example, solid or non-solid. Tumors can be, for example, benign (that is, not cancerous), pre-maglignant (that is, pre-cancerous), or maliganant (that is, cancerous). The term “solid tumor” as used herein refers to an abnormal mass of tissue that usually does not contain cysts or liquid areas. Solid tumors may be benign, pre-galignant, or malignant. Different types of solid tumors sometimes are named for the type of cells that form them. Solid tumor can occur in various places, for example, bones, muscle, and organs. Examples of solid tumors include, but are not limited to, sarcomas, carcinomas, lymphomas, and combinations thereof. Sarcomas are generally known as tumors in a blood vessel, bone, fat tissue, ligament, lymph vessel, muscle or tendon, for example Ewing sarcoma, osteosarcoma, and Rhabdomyosarcoma. Carcinomas are generally known as tumors that form in epithelia cells, for example epithelia cells found in skin, glands and the linings of organs (including but not limited to bladder, ureters, and kidneys). Non-limiting examples of carcinomas include adrenocortical carcinoma. Non-solid tumors are sometime referred to as dispersed tumors, for example, tumors in the blood (also known as leukemia). Non-limiting examples of non-solid tumors include hematological malignancy, leukemias, lymphomas (for example, Hodgkin's disease, Non-Hodgkin's lymphoma). Examples of tumors include, but are not limited to, cervical cancer, colon cancer, liver cancer, prostate cancer, melanoma, ovarian cancer, lung cancer, renal cell carcinoma, Schwannoma, mesothelioma, acute myeloid leukemia, multiple myeloma, non-Hodgkin lymphoma, or a combination thereof.

Throughout this disclosure, various aspects are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present disclosure will become apparent from the following specification taken in conjunction with the accompanying drawings.

In the following description, numerous specific details are set forth to provide a more thorough understanding of the present disclosure. However, it will be apparent to one of skill in the art that the methods of the present disclosure may be practiced without one or more of these specific details. In other instances, well-known features and procedures well known to those of skill in the art have not been described in order to avoid obscuring the disclosure.

### SerRS Proteins and Polynucleotides

Seryl-tRNA synthetase (SerRS; also known as Serine-tRNA ligase) is an enzyme that belongs to the class II aminoacyl tRNA synthetase (aaRS) family. aaRS is an enzyme that attaches the appropriate amino acid onto its tRNA. It does so by catalyzing the esterification of a specific cognate amino acid or its precursor to one of all its compatible cognate tRNAs to form an aminoacyl-tRNA. SerRS catalyzes the aminoacylation reaction that charges serine onto its cognate tRNA for protein synthesis. This evolutionarily conserved essential reaction happens in two steps: (1) serine is activated by ATP to form serine-adenylate (Ser-AMP) as the enzyme-bound reaction intermediate; and (2) the seryl moiety on Ser-AMP is transferred to the 3′ of the cognate tRNA to generate the final product Ser-tRNA^{Ser} to be delivered to the ribosome. As described herein, it is a surprise for SerRS to be identified as a transcriptional suppressor of angiogenesis, a hallmark in cancer development.

The vertebrate SerRS enzyme is encoded by the SARS gene, which is evolutionarily related to bacterial and yeast counterparts. Non-limiting examples of the vertebrate SerRS proteins include human SerRS, mouse SerRS, zebrafish SerRS, and Frog SerRS. The coding sequence (CDS) of the human, mouse, zebrafish, and frog SARS gene is shown in SEQ ID NO: 39, 41, 43, and 45, respectively. Also disclosed herein are nucleotide sequences that comprise, or consist of, a SerRS nucleotide sequence having at least 70%, at least 75%, at least 80%, at least 85%, at least 90%, at least 95%, at least 98%, or at least 99%, sequence identity to SEQ ID NO: 39, SEQ ID NO: 41, SEQ ID NO: 43, or SEQ ID NO: 45. In some embodiments, the SerRS nucleotide sequence is, or is about, 100% identical to SEQ ID NO: 39, SEQ ID NO: 41, SEQ ID NO: 43, or SEQ ID NO: 45. In some embodiments, the SerRS nucleotide sequence comprises or consists of the nucleotide sequence of SEQ ID NO: 40 which encodes SerRS^{S101A/S241A} protein.

The amino acid sequence of wildtype human SerRS protein is shown below (SEQ ID NO: 1). The amino acid sequence of wildtype mouse, zebrafish and frog SerRS protein is provided in SEQ ID NO: 42, 44, and 46, respectively. Also disclosed herein are proteins that comprise, or consist of, a SerRS protein sequence having at least 70%, at least 75%, at least 80%, at least 85%, at least 90%, at least 95%, at least 98%, or at least 99%, sequence identity to SEQ ID NO: 1, SEQ ID NO: 42, SEQ ID NO: 44, or SEQ ID NO: 46. In some embodiments, the SerRS protein sequence is, or is about, 100% identical to SEQ ID NO: 1, SEQ ID NO: 42, SEQ ID NO; 44, or SEQ ID NO: 46.

(SEQ ID NO. 1)
MVLDLDLFRVDKGGDPALIRETQEKRFKDPGLVDQLVKADSEWRRCRFRA
DNLNKLKNLCSKTIGEKMKKKEPVGDDESVPENVLSFDDLTADALANLKV
SQIKKVRLLIDEAILKCDAERIKLEAERFENLREIGNLLHPSVPISNDED
VDNKVERIWGDCTVRKKYSHVDLVVMVDGFEGEKGAVVAGSRGYFLKGVL
VFLEQALIQYALRTLGSRGYIPIYTPFFMRKEVMQEVAQLSQFDEELYKV
IGKGSEKSDDNSYDEKYLIATSEQPIAALHRDEWLRPEDLPIKYAGLSTC
FRQEVGSHGRDTRGIFRVHQFEKIEQFVYSSPHDNKSWEMFEEMITTAEE
FYQSLGIPYHIVNIVSGSLNHAASKKLDLEAWFPGSGAFRELVSCSNCTD
YQARRLRIRYGQTKKMMDKVEFVHMLNATMCATTRTICAILENYQTEKGI
TVPEKLKEFMPPGLQELIPFVKPAPIEQEPSKKQKKQHEGSKKKAAARDV
TLENRLQNMEVTDA

SerRS Proteins with Modified Phosphorylation Capabilities and Polynucleotides Thereof

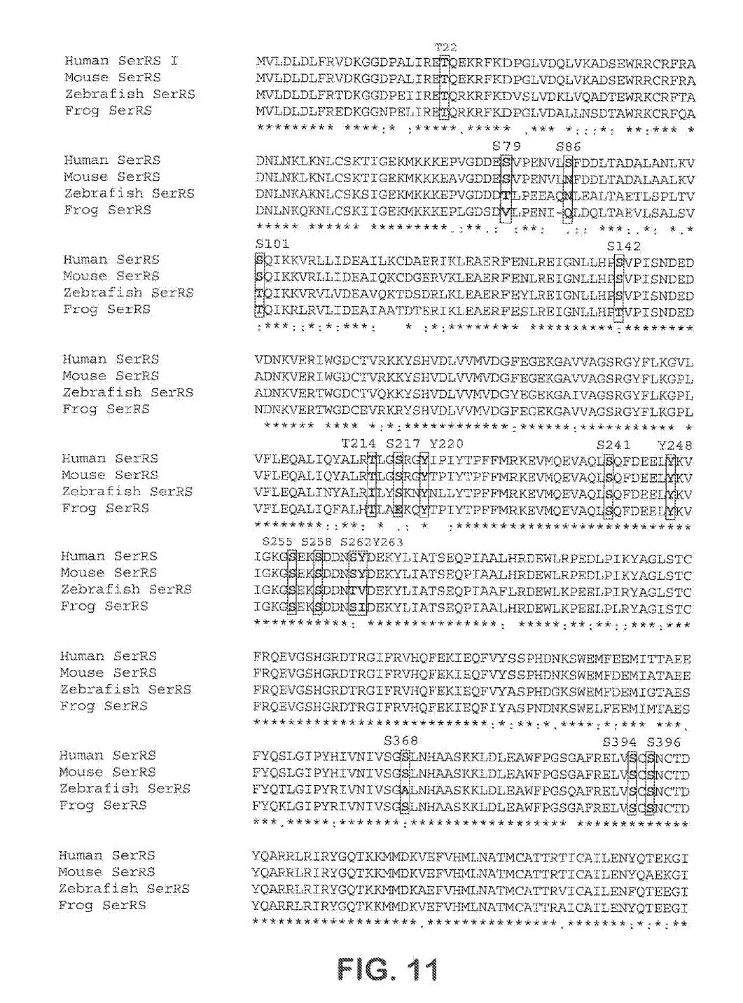

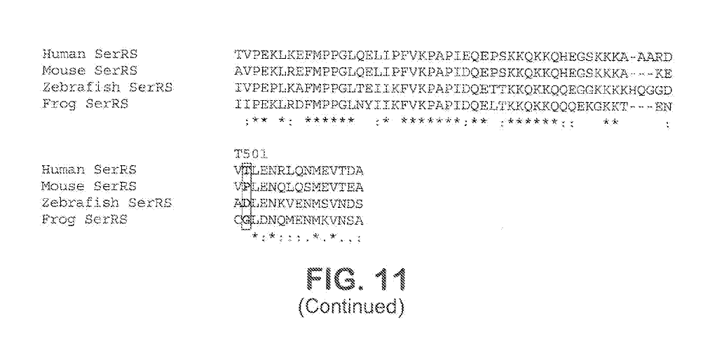

Various phosphorylation sites have been found in SerRS proteins. For example, non-limiting phosphorylation sites in the wildtype human SerRS protein (SEQ ID NO: 1) include T22, S79, S86, S101, S142, S217, S241, S255, S258, S262, S368, S394, S396, T214, T501, Y220, Y248, and Y263. These serine (S), threonine (T) and tyrosine (Y) phosphorylation sites have been found to be highly conserved in vertebrate SerRS proteins, but may vary in the non-human SerRS proteins. For example, as exemplified in FIG. 11, in some vertebrates, one or more of the serine at these phosphorylation sites may be threonine, tyrosine, alanine, or valine in the SerRS protein; one or more of the threonine at these phosphorylation sites may be serine, tyrosine, alanine, or valine in the SerRS protein; and one or more of the tyrosine at these phosphorylation sites in human SerRS protein may be threonine, serine, alanine, or valine in the SerRS protein. For example, in frog and zebrafish SerRS protein, the corresponding residue to S101 in human SerRS protein is T, and in frog SerRS protein, the corresponding residue to S142 in human SerRS protein is T (see FIG. 11). In the present disclosure, the position of an amino acid in a SerRS protein is referred as the position of the corresponding amino acid in the human SerRS protein. For example, sequence alignment of one or more SerRS proteins of interest and the wildtype human SerRS protein (e.g., the one shown in FIG. 11) can be used to determine the position of one or more amino acids in the SerRS protein of interest. In some embodiments, the SerRS proteins disclosed herein are capable of being phosphorylated, for example by ataxia telangiectasia mutated kinase (ATM), ataxia telangiectasia and Rad3-related kinase (ATR), or both. Without being bound by any particular theory, it is believed that the extent of phosphorylation of a SerRS protein can be modulated (for example, reduced or enhanced) by making amino acid substitutions, deletions, additions, or combinations thereof at or near one or more of the phosphorylation sites on the SerRS protein. For example, variant SerRS proteins (for example, mutant SerRS proteins) can be generated by making amino acid substitutions, deletions, additions, or combinations thereof at or near one or more of the phosphorylation sites on the corresponding parent SerRS protein (for example, a wildtype SerRS protein).

Some embodiments disclosed herein provide variant SerRSs proteins (for example, mutant SerRS proteins) that are phosphorylation-deficient as compared to the corresponding parent SerRS protein (for example, a wildtype SerRS protein). As disclosed herein, a variant SerRS protein is considered to be phosphorylation-deficient if the maximum level of phosphorylation of the variant SerRS protein is less than 90%, 85%, 80%, 75%, 70%, 65%, 60% 50%, 45%, 40%, 35%, 30%, 25%, 20%, 15%, 10%, 5%, 4%, 3%, 2%, or 1% of that of the corresponding parent SerRS protein (for example, a wildtype SerRS protein) or that of the human wildtype SerRS protein (for example, a SerRS protein having the sequence of SEQ ID NO: 1). In some embodiments, the maximum level of phosphorylation of the variant SerRS protein is, or is about, 90%, 85%, 80%, 75%, 65%, 60%, 50%, 45%, 40%, 35%, 30%. 25%, 20%, 15%, 10%, 5%, 4%, 3%, 2%, 1%, 0.5%, or a range between any two of these values, of that of the corresponding parent SerRS protein (for example, a wildtype SerRS protein). In some embodiments, the maximum level of phosphorylation of the variant SerRS protein is, or is about, 90%, 85%, 80%, 75%, 65%, 60%, 50%, 45%, 40%, 35%, 30%, 25%, 20%, 15%, 10%, 5%, 4%, 3%, 2%, 1%, 0.5%, or a range between any two of these values, of that of human wildtype SerRS protein (for example, the SerRS protein having the sequence of SEQ ID NO: 1). In some embodiments, the variant SerRS protein cannot be phosphorylated. Also as disclosed herein, a variant SerRS protein is considered to be phosphorylation-deficient if the average level of phosphorylation of the variant SerRS protein is less than 90%, 85%, 80%, 75%, 70%, 65%, 60% 50%, 45%, 40%, 35%, 30%, 25%, 20%, 15%, 10%, 5%, 4%, 3%, 2%, or 1% of that of the corresponding parent SerRS protein (for example, a wildtype SerRS protein) or that of the human wildtype SerRS protein (for example, a SerRS protein having the sequence of SEQ ID NO: 1). In some embodiments, the average level of phosphorylation of the variant SerRS protein is, or is about, 90%, 85%, 80%, 75%, 65%, 60%, 50%, 45%, 40%, 35%, 30%, 25%, 20%, 15%, 10%, 5%, 4%, 3%, 2%, 1%, 0.5%, or a range between any two of these values, of that of the corresponding parent SerRS protein (for example, a wildtype SerRS protein). In some embodiments, the average level of phosphorylation of the variant SerRS protein is, or is about, 90%, 85%, 80%, 75%, 65%, 60%, 50%, 45%, 40%, 35%, 30%, 25%, 20%, 15%, 10%, 5%, 4%, 3%, 2%, 1%, 0.5%, or a range between any two of these values, of that of human wildtype SerRS protein (for example, a SerRS protein having the sequence of SEQ ID NO: 1).

In some embodiments, the variant SerRS protein comprise an amino acid substitution at one or more of residue position 22, 79, 86, 101, 142, 217, 241, 255, 258, 262, 368, 394, 396, 214, 501, 220, 248, and 263 corresponding to the relative parent SerRS protein or a wildtype SerRS protein (e.g., a human wildtype SerRS protein). For example, the variant SerRS protein comprise an amino acid substitution at one or more of residue corresponding to T22, S79, S86, S101, S142, S217, S241, S255, S258, S262, S368, S394, S396, T214, T501, Y220, Y248, and Y263 of human wildtype SerRS protein. In some embodiments, the variant SerRS protein comprise an amino acid substitution at one or more of residues T22, S79 (or T79), S86, S101 (or TI 01), S142 (or T142), S217, S241, S255, S258, S262 (or 1262), S368, S394, S396, T214, T501, Y220 (or T220), Y248, and Y263 relative to the corresponding parent SerRS protein or a wildtype SerRS protein (e.g., a human wildtype SerRS protein). The amino acid substitution can be, for example, serine-to-alanine, serine-to-glycine, serine-to-lysine, serine-to-arginine, serine-to-asparagine, serine-to-glutamine, serine-to-histidine, serine-to-cysteine, serine-to-valine, serine-to-leucine, serine-to-isoleucine, serine-to-proline, serine-to-methionine, serine-to-tryptophan, serine-to-phenylalanine, threonine-to-alanine, threonine-to-glycine, threonine-to-lysine, threonine-to-arginine, threonine-to-asparagine, threonine-to-glutamine, threonine-to-histidine, threonine-to-cysteine, threonine-to-valine, threonine-to-leucine, threonine-to-isoleucine, threonine-to-proline, threonine-to-methionine, threonine-to-tryptophan, threonine-to-phenylalanine, tyrosine-to-alanine, tyrosine-to-glycine, tyrosine-to-lysine, tyrosine-to-arginine, tyrosine-to-asparagine, tyrosine-to-glutamine, tyrosine-to-histidine, tyrosine-to-cysteine, tyrosine-to-valine, tyrosine-to-leucine, tyrosine-to-isoleucine, tyrosine-to-proline, tyrosine-to-methionine, tyrosine-to-tryptophan, and tyrosine-to-phenylalanine. As disclosed herein, the variant SerRS protein can comprise one, two, three, four, five, six, seven, eight, nine, ten, or more, amino acid substitutions as compared to the corresponding parent SerRS protein or a wildtype SerRS protein (e.g., a human wildtype SerRS protein). As disclosed herein, the variant SerRS protein can be, or be about, 70%, 75%, 80%, 85%, 90%, 95%. 98%, 99%, or more identical in sequence as compared to the corresponding parent SerRS protein or a wildtype SerRS protein (e.g., a human wildtype SerRS protein). In some embodiments, the parent SerRS protein is a human SerRS protein. In some embodiments, the parent SerRS protein is a human wildtype SerRS protein (for example, the SerRS protein having the sequence of SEQ 1D NO: 1). In some embodiments, the variant SerRS protein comprises an amino acid substitution at Serine 101 (S101), Serine 241 (S241), or both relative to the corresponding parent SerRS protein (for example, human wildtype SerRS protein (e.g., the SerRS protein having the sequence of SEQ ID NO: 1), and a human variant SerRS protein).

In some embodiments, the variant SerRS protein comprise an amino acid deletion at one or more of residue position 22, 79, 86, 101, 142, 217, 241, 255, 258, 262, 368, 394, 396, 214, 501, 220, 248, and 263 corresponding to the relative parent SerRS protein or a wildtype SerRS protein (e.g., a human wildtype SerRS protein). For example, the variant SerRS protein comprise an amino acid deletion at one or more of residue corresponding to T22, S79, S86, S101, S142, S217, S241, S255, S258, S262, S368, S394, S396, T214, T501, Y220, Y248, and Y263 of human wildtype SerRS protein. In some embodiments, the variant SerRS protein comprises an amino acid deletion at one or more of residues T22, S79, S86, S101 (or T101), S142, S217, S241, S255, S258, S262, S368, S394, S396, T214, T501, Y220, Y248, and Y263 relative to the corresponding parent SerRS protein (for example, human wildtype SerRS protein (e.g., the SerRS protein having the sequence of SEQ ID NO: 1)). As disclosed herein, the variant SerRS protein can comprise one, two, three, four, five, six, seven, eight, nine, ten, or more, amino acid deletions as compared to the corresponding parent SerRS protein. As disclosed herein, the variant SerRS protein can be about 70%, 75%, 80%, 85%, 90%, 95%, 98%, 99%, or more identical in sequence as compared to the corresponding parent SerRS protein. In some embodiments, the parent SerRS protein is a human SerRS protein. In some embodiments, the parent SerRS protein is a human wildtype SerRS protein (for example, the SerRS protein having the sequence of SEQ ID NO: 1). In some embodiments, the variant SerRS protein comprises an amino acid deletion at serine 101 (S101), threonine 101 (T101) or serine 241 (S241), or both relative to the corresponding parent SerRS protein (for example, human wildtype SerRS protein (SEQ ID NO: 1)).

As disclosed herein, the parent SerRS protein can be a vertebrate protein, for example a mammalian protein (including but not limited to a human protein). In some embodiments, the variant SerRS protein is a vertebrate protein, for example a human protein.

As a non-limiting example, a human SerRS protein (for example, the human wildtype SerRS protein having the sequence of SEQ ID NO: 1) can be modified to reduce its ability to be phosphorylated. For example, one or more of residues T22, S79, S86, S101 (or T01), S142, S217, S241, S255, S258, S262, S368, S394, S396, T214, T501, Y220, Y248, and Y263 of SEQ ID NO: 1 can be substituted, deleted, or both, to generate mutant human SerRS proteins whose maximum or average level of phosphorylation is reduced as compared to the parent human SerRS protein (including but not limited to the human wildtype SerRS protein). In some embodiments, the mutant SerRS protein comprises or consists of an amino acid sequence having at least 90%, at least 95%, at least 98%, at least 99%, identity to the amino acid sequence set forth in SEQ ID NO:1 and comprises an amino acid deletion at one or more of residues T22, S79, S86, S101 (or T101), S142, S217, S241, S255, S258, S262, S368, S394, S396, T214, T501, Y220, Y248, and Y263 of SEQ ID NO: 1. In some embodiments, the amino acid deletion is at one or both of S101 and S241. In some embodiments, the mutant SerRS protein comprises or consists of an amino acid sequence having at least 80%, at least 85%, at least 90%, at least 95%, at least 98%, or at least 99% sequence identity to the amino acid sequence set forth in SEQ ID NO:1 and comprises an amino acid substitution at one or more of residues T22, S79, S86, S101 (or T101), S142, S217, S241, S255, S258, S262, S368, S394, S396, T214, T501, Y220, Y248, and Y263 of SEQ ID NO: 1. In some embodiments, the amino acid substitution is at one or both of S101 and S241. In some embodiments, the amino acid substitution is one or more of serine-to-alanine, serine-to-glycine, serine-to-lysine, serine-to-arginine, serine-to-asparagine, serine-to-glutamine, serine-to-histidine, serine-to-cysteine, serine-to-valine, serine-to-leucine, serine-to-isoleucine, serine-to-proline, serine-to-methionine, serine-to-tryptophan, serine-to-phenylalanine, threonine-to-alanine, threonine-to-glycine, threonine-to-lysine, threonine-to-arginine, threonine-to-asparagine, threonine-to-glutamine, threonine-to-histidine, threonine-to-cysteine, threonine-to-valine, threonine-to-leucine, threonine-to-isoleucine, threonine-to-proline, threonine-to-methionine, threonine-to-tryptophan, threonine-to-phenylalanine, tyrosine-to-alanine, tyrosine-to-glycine, tyrosine-to-lysine, tyrosine-to-arginine, tyrosine-to-asparagine, tyrosine-to-glutamine, tyrosine-to-histidine, tyrosine-to-cysteine, tyrosine-to-valine, tyrosine-to-leucine, tyrosine-to-isoleucine, tyrosine-to-proline, tyrosine-to-methionine, tyrosine-to-tryptophan, and tyrosine-to-phenylalanine. In some embodiments, the mutant SerRS protein comprises an amino acid sequence having at least 80%, at least 85%, at least 90%, at least 95%, at least 98%, at least 99%, identity to the amino acid sequence set forth in SEQ ID NO: 1 and comprises an amino acid substitution at one or both of residue S101 and S241 in SEQ ID NO: 1, wherein the amino acid substitution is serine-to-alanine or serine-to-glycine. Non-limiting examples of mutant SerRS proteins includes proteins comprising, or consisting of, an amino acid sequence set forth in SEQ ID NO: 2 (human SerRS^{S101A} mutant), SEQ ID NO: 3 (human SerRS^{S241A} mutant) or SEQ ID NO: 4 (human SerRS^{S101A/S241A} mutant). In some embodiments, the mutant SerRS protein is at least 80%, at least 85%, at least 90%, at least 95%, at least 98%, at least 99%, identical in sequence as compared to the sequence set forth in SEQ ID NO: 2, SEQ ID NO: 3 or SEQ ID NO: 4.

In some embodiments, the variant SerRS protein comprise an amino acid substitution at one or more of residue position 22, 79, 86, 101, 142, 217, 241, 255, 258, 262, 368, 394, 396, 214, 501, 220, 248, and 263 corresponding to the relative parent SerRS protein or a wildtype SerRS protein (e.g., a human wildtype SerRS protein) and an amino acid deletion at one or more of residue positions 22, 79, 86, 101, 142, 217, 241, 255, 258, 262, 368, 394, 396, 214, 501, 220, 248, and 263 corresponding to the relative parent SerRS protein or a wildtype SerRS protein (e.g., a human wildtype SerRS protein). For example, the variant SerRS protein comprise an amino acid substitution at one or more of residue corresponding to T22, S79, S86, S101, S142, S217, S241, S255, S258, S262, S368, S394, S396, T214, T501, Y220, Y248, and Y263 of human wildtype SerRS protein and an amino acid deletion at one or more of residue corresponding to T22, S79, S86, S101, S142, S217, S241, S255, S258, S262, S368, S394, S396, T214, T501, Y220, Y248, and Y263 of human wildtype SerRS protein. In some embodiments, the phosphorylation-deficient variant SerRS protein at least one amino acid deletion and at least one amino acid substitution at residues T22, S79, S86, S101 (or T101), S142, S217, S241, S255, S258, S262, S368, S394, S396; T214, T501, Y220, Y248, and Y263 relative to the corresponding parent SerRS protein. In some embodiments, the phosphorylation-deficient variant SerRS protein at least one amino acid deletion and at least one amino acid substitution at residues T22, S79, S86, S101 (or T101), S142, S217, S241, S255, S258, 5262, S368, S394, S396; T214, T501, Y220, Y248, and Y263 relative to human wildtype SerRS protein (for example, the SerRS protein having the sequence of SEQ ID NO: 1) or a variant thereof.

Some embodiments disclosed herein provide variant SerRS proteins (for example, mutant SerRS proteins) that are constitutively phosphorylated or mimic phosphorylated SerRS proteins. In some embodiments, the variant SerRS protein cannot be dephosphorylated. In some embodiments, the variant SerRS protein is deficient in repressing VEGF transcription as compared to the corresponding parent SerRS protein. For example, a mutant SerRS protein can be deficient in repressing VEGF transcription as compared to the corresponding parent SerRS protein (e.g., the corresponding wildtype SerRS protein) or variant thereof. For example, the extent that the variant SerRS protein represses VEGF transcription can be, or be about, 90%, 85%, 80%, 75%, 70%, 60%, 50%, 40%, 30%, 20%, 10%, 5%, 4%, 3%, 2%, 1%, or a range between any two of these values, of that of the corresponding parent SerRS protein (e.g., a wildtype SerRS protein). In some embodiments, the extent that the variant SerRS protein can repress VEGF transcription is less than 90%, 85%, 80%, 75%, 70%, 60%, 50%, 40%, 30%, 20%, 10%, 5%, 4%, 3%, 2%, or 1%, of that of the corresponding parent SerRS protein. In some embodiments, the extent that the variant SerRS protein can repress VEGF transcription is less than 90%, 85%, 80%, 75%, 70%, 60%, 50%, 40%, 30%, 20%, 10%, 5%, 4%, 3%, 2%, or 1%, of that of wildtype human SerRS protein (for example, the SerRS protein having the sequence of SEQ ID NO: 1) or a variant thereof. In some embodiments, the variant SerRS protein does not repress VEGF transcription. In some embodiments, the variant SerRS protein repress VEGF transcription by no more than 50%, 40%, 30%, 20%, 10%, 5%, 4%, 3%, 2%, or 1%.

In some embodiments, the variant SerRS protein comprise an amino acid substitution at one or more of residues T22, S79 (or T79), S86, S101 (or T101), S142 (or T142), S217, S241, S255, S258, S262, S368, S394, S396, T214, T501, Y220 (or T220), Y248, and Y263 relative to the corresponding parent SerRS protein (e.g., a wildtype SerRS protein). As disclosed herein, the variant SerRS protein can comprise one, two, three, four, five, six, seven, eight, nine, ten, or more, amino acid substitutions as compared to the corresponding parent SerRS protein. As disclosed herein, the variant SerRS protein can be about 80%, 85%, 90%, 95%, 98%, 99%, or more identical in sequence as compared to the corresponding parent SerRS protein. In some embodiments, the parent SerRS protein is a human SerRS protein. In some embodiments, the parent SerRS protein is human wildtype SerRS protein (for example, the SerRS protein having the sequence of SEQ ID NO: 1) or a variant thereof. In some embodiments, the variant SerRS protein comprises an amino acid substitution at Serine 101 (S101), Serine 241 (S241), or both relative to the corresponding parent SerRS protein (for example, human wildtype SerRS protein (SEQ ID NO: 1) or a variant thereof).

As disclosed herein, the parent SerRS protein can be a vertebrate protein, for example a mammalian protein (including but not limited to a human, mouse, zebrafish, or frog protein). In some embodiments, the variant SerRS protein is a vertebrate protein, for example a human, mouse, zebrafish, or frog protein.

As a non-limiting example, human wildtype SerRS protein (for example, the SerRS protein having the sequence of SEQ ID NO: 1) can be modified to enhance the extent of its phosphorylation. For example, one or more of residues T22, S79, S86, S101, S142, S217, S241, S255, S258, S262, S368, S394, S396, T1214, T501, Y220, Y248, and Y263 of SEQ ID NO: 1 can be substituted to generate human mutant SerRS proteins with reduced ability to be phosphorylated. In some embodiments, the mutant SerRS protein comprises or consists of an amino acid sequence having at least 80%, at least 85%, at least 90%, at least 95%, at least 98%, at least 99%, identity to the amino acid sequence set forth in SEQ ID NO:1 and comprises an amino acid substitution at one or more of residues T22, S79, S86, S101, S142, S217, S241, S255, S258, S262, S368, S394, S396, T214, T501, Y220, Y248, and Y263 of SEQ ID NO: 1. In some embodiments, the amino acid substitution is at one or both of S101 and S241. In some embodiments, the amino acid substitution is serine-to-aspartic acid or serine-to-glutamic acid. In some embodiments, the mutant SerRS protein comprises an amino acid sequence having at least 80%, at least 85%, at least 90%, at least 95%, at least 98%, at least 99%, identity to the amino acid sequence set forth in SEQ ID NO: 1 and comprises an amino acid substitution at one or both of residue S101 and S241 in SEQ ID NO: 1, wherein the amino acid substitution is serine-to-aspartic acid or serine-to-glutamic acid. Non-limiting examples of mutant SerRS proteins includes proteins comprising, or consisting of, an amino acid sequence set forth in SEQ ID NO: 5 (human SerRS^{S241D} mutant) or SEQ ID NO: 6 (human SerRS^{S241E} mutant). In some embodiments, the mutant SerRS protein is at least 90%, at least 95%, at least 98%, at least 99%, identical in sequence as compared to the sequence set forth in SEQ ID NO: 5 or SEQ ID NO: 6.

In some embodiments, the parent SerRS protein is not a naturally occurring protein. For example, the parent SerRS can be a chimeric protein comprising sequences from two, three, four, five, six, seven, eight, nine, ten, or more different SerRS proteins. In some embodiments, the parent SerRS is a chimeric protein comprising sequences from human SerRS protein and one or more other mammalian SerRS proteins (for example, mouse SerRS protein and rat SerRS protein). In some embodiments, the parent SerRS is a chimeric protein comprising sequences from human SerRS protein and one or more vertebrate SerRS proteins (for example, mouse SerRS protein, rat SerRS protein, Zebrafish SerRS protein, or frog SerRS protein). In some embodiments, the parent SerRS is a chimeric protein comprising sequences from human SerRS protein and one or more invertebrate SerRS proteins (for example, yeast SerRS protein and E. coli SerRS protein). In some embodiments, the parent SerRS is a chimeric protein comprising sequences from human SerRS protein and one or more plant SerRS proteins (for example, Arabidopsis thaliana SerRS protein). It has been shown that protein sequences are evolutionarily conserved for SerRS proteins. In some embodiments, the parent SerRS protein comprises a one or more consensus sequences obtained by aligning parts or the entire sequence of two or more different SerRS proteins. For example, a consensus sequence can be built by aligning human, yeast and E. coli SerRS sequences. As another example, a consensus sequence can be built by aligning two or more vertebrate SerRS sequences (including but not limited to mouse, human, frog, and/or zebrafish SerRS sequences). One or more parts of this consensus sequence (for example, the conserved regions identified) can be used to replace the corresponding sequence in the wildtype human SerRS to generate a parent SerRS protein. In some embodiments, a protein comprising, or consisting of, the consensus sequence is used as the parent SerRS protein.

The variant SerRS proteins disclosed herein may contain conservative amino acid substitution(s) at one or more locations along their sequences as compared to a reference SerRS protein. Non-limiting examples of the reference SerRS protein include the corresponding parent SerRS protein, human wildtype SerRS protein (for example, the SerRS protein having the sequence of SEQ ID NO: 1) or a variant thereof. As used herein, a “conservative amino acid substitution” is one in which the amino acid residue is replaced with an amino acid residue having a similar side chain. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. In some embodiments, replacement of a leucine with an isoleucine or valine, an aspartate with a glutamate, a threonine with a serine, or a similar replacement of an amino acid with a structurally related amino acid is not expected to have a major effect on the properties of the resulting variant polypeptide. Whether an amino acid change results in a functional polypeptide can readily be determined by assaying its activity, as described herein. Exemplary conservative amino acid substitutions are shown in Table 1. Amino acid substitutions falling within the scope of the present disclosure, are, in general, accomplished by selecting substitutions that do not differ significantly in their effect on maintaining (a) the structure of the peptide backbone in the area of the substitution, (b) the charge or hydrophobicity of the molecule at the target site, (c) the bulk of the side chain, or (d) the biological function. After the substitutions are introduced, the variants are screened for biological activity.

TABLE 1
Exemplary conservative amino acid substitution

| Original | Exemplary conservative |
|---|---|
| Residue | Substitution |
| Ala | Val, Leu, Ile |
| Arg | Lys, Gln, Asn |
| Asn | Gln, His, Lys, Arg |
| Asp | Glu |
| Cys | Ser |
| Gln | Asn, His, Lys |
| Glu | Asp, Lys |
| Gly | Pro |
| His | Asn, Gln, Lys, Arg |
| Ile | Leu, Val, Met, Ala, Phe, |
| | Norleu |
| Leu | Norleu, Ile, Val, Met, Ala, |
| | Phe |
| Lys | Arg, Gln, Asn |
| Met | Leu, Ile, Phe |
| Phe | Leu, Val, Ile, Ala |
| Pro | Gly |
| Ser | Thr |
| Thr | Ser |
| Trp | Tyr |
| Tyr | Trp, Phe, Thr, Ser |
| Val | Ile, Leu, Met, Phe, Ala, |
| | Norleu |

Also disclosed herein are polynucleotide sequences comprising, or consisting of, coding sequence of any of the SerRS proteins (including the wildtype and variant SerRS proteins) disclosed herein.

### Expression of SerRS Proteins

SerRS proteins suitable for the embodiments of the present disclosure can be produced, for example, by recombinant DNA technology in various host cells. For example, expression vectors capable of expressing eukaryotic proteins (e.g., viral vectors, shuttle vectors, and bacterial plasmids) can be used to express the recombinant SerRS proteins. In some embodiments, the host cells can be bacterial, fungal, plant, yeast, insect or vertebrate cells (including but not limited to mammalian cells). The term “host cell” includes both the cells, progeny of the cells and protoplasts created from the cells that are used to produce a SerRS protein according to the disclosure. In some embodiments, the host cells are prokaryotic cells, for example bacteria host cells.

As a non-limiting example, to produce the SerRS protein with the recombinant DNA technology, a DNA construct comprising nucleic acid encoding the amino acid sequence of the SerRS protein can be constructed and transferred into, for example, an E. coli host cell. The vector may be any vector which when introduced into an E. coli host cell can be integrated into the host cell genome and can be replicated. The nucleic acid encoding the SerRS can be operably linked to a suitable promoter, which shows transcriptional activity in E. coli host cell. The promoter may be derived from genes encoding proteins either homologous or heterologous to the host cell. As used herein, an “inducible promoter” may refer to a promoter that is active under environmental or developmental regulation.

In some embodiments, the SerRS coding sequence can be operably linked to a signal sequence. In some embodiments, the expression vector may also include a termination sequence. In some embodiments, the termination sequence and the promoter sequence can be derived from the same source. In another embodiment, the termination sequence can be homologous to the host cell.

In some embodiments, the expression vector includes one or more selectable markers. Examples of representative selectable markers include ones that confer antimicrobial resistance (e.g., hygromycin and phleomycin). In some embodiments, nutritional selective markers including those markers known in the art as amdS, argB, and pyr4, can be used as the selectable marker.

An expression vector comprising a DNA construct with a polynucleotide encoding the SerRS may be any vector which is capable of replicating autonomously in a given host organism or of integrating into the DNA of the host. In some embodiments, the expression vector can be a plasmid or a viral construct.

In some embodiments, two types of expression vectors for obtaining expression of genes are contemplated. For example, the first expression vector may comprise DNA sequences in which the promoter, SerRS-coding region, and terminator all originate from the gene to be expressed. In some embodiments, gene truncation can be obtained by deleting undesired DNA sequences (e.g., DNA encoding unwanted domains) to leave the domain to be expressed under control of its own transcriptional and translational regulatory sequences. The second type of expression vector may be preassembled and contains sequences needed for high-level transcription and a selectable marker. In some embodiments, the coding region for the SARS gene or part thereof can be inserted into this general-purpose expression vector such that it is under the transcriptional control of the expression construct promoter and terminator sequences. In some embodiments, genes or part thereof may be inserted downstream of a strong promoter.

Methods used to ligate the DNA construct comprising a polynucleotide encoding the SerRS, a promoter, a terminator and other sequences and to insert them into a suitable vector are well known in the art. Linking can be generally accomplished by ligation at convenient restriction sites. If such sites do not exist, the synthetic oligonucleotide linkers are used in accordance with conventional practice (Bennett & Lasure, More Gene Manipulations in Fungi, Academic Press, San Diego (1991) pp 70-76). Additionally, vectors can be constructed using known recombination techniques (e.g., Invitrogen Life Technologies, Gateway Technology).

Introduction of a DNA construct or vector into a host cell includes techniques such as transformation; electroporation; nuclear microinjection; transduction; transfection, (e.g., lipofection mediated and DEAE-Dextrin mediated transfection); incubation with calcium phosphate DNA precipitate; high velocity bombardment with DNA-coated microprojectiles; and protoplast fusion. General transformation techniques are known in the art (see, e.g., Campbell et al., (1989) Curr. Genet. 16:53-56).

In some embodiments, genetically stable transformants can be constructed with vector systems whereby the nucleic acid encoding SerRS is stably integrated into a host strain chromosome. Transformants can then be purified by known techniques.

### Methods and Compositions for Reducing Tumor Progression

Disclosed herein are methods and compositions for reducing tumor progression. The method, in some embodiments, comprises, administering a composition comprising a mutant SerRS protein to a subject in need, wherein the mutant SerRS protein is a phosphorylation-deficient mutant SerRS protein, whereby tumor progression is reduced in the subject. For example, the maximum and/or average level of phosphorylation of the mutant SerRS protein is, or is about, 50%, 40%, 30%, 20%, 10%, 5%, 3%, 1%, or a range between any two of these values, of that of the corresponding parent SerRS protein or that of the corresponding wildtype SerRS protein. In some embodiments, the maximum and/or average level of phosphorylation of the mutant SerRS protein is less than 50%, less than 40%, less than 30%, less than 20%, less than 10%, less than 5%, less than 3%, less than 1%, of that of the corresponding parent SerRS protein or that of the corresponding wildtype SerRS protein. In some embodiments, the maximum and/or average level of phosphorylation of the mutant SerRS protein is less than 50%, less than 40%, less than 30%, less than 20%, less than 10%, less than 5%, less than 3%, less than 1%, of that of human wildtype SerRS protein (for example, the SerRS protein having the sequence of SEQ ID NO: 1).

The composition can be, for example, a pharmaceutical composition. In some embodiments, the pharmaceutical composition comprises one or more inhibitors of hypoxia-inducible factor (HIF), for example one or more inhibitor of HIF-1. In some embodiments, the pharmaceutical composition does not comprise any inhibitors of HIF, for example HIF-1 inhibitors. In some embodiments, tumor progression is reduced by 10%, 20%, 30%, 40%, 50%, 60%, 70%, 80%, 90%, 95%, 98%, 99%, or a range between any two of these values, as compared to the subjects received no treatment. Without being bound by any particular theory, it is believed that phosphorylated SerRS proteins can repress transcription of vascular endothelial growth factor (VEGF) in the subject, which can lead to a reduction in angiogenesis. In some embodiments, the reduction in tumor progression is achieved by reducing angiogenesis in the subject. For example, the angiogenesis in the subject may be reduced by 10%, 20%, 30%, 40%, 50%, 60%, 70%, 80%, 90%, 95%, 98%, 99%, or a range between any two of these values as compared to the subjects received no treatment. In some embodiments, the angiogenesis in the subject is reduced by at least 10%, at least 20%, at least 30%, at least 40%, at least 50%, at least 60%, at least 70%, at least 80%, at least 90%, at least 95%, at least 98%, or at least 99%, as compared to the subjects received no treatment. In some embodiments, the angiogenesis in the subject is reduced by more than 10%, more than 20%, more than 30%, more than 40%, more than 50%, more than 60%, more than 70%, more than 80%, more than 90%, more than 95%, more than 98%, or more than 99%, as compared to the subjects received no treatment. In some embodiments, the angiogenesis is hypoxia-induced angiogenesis. In some embodiments, the tumor progression is metastasis. In some embodiments, the solid tumor is sarcomas, carcinomas, lymphomas, or a combination thereof. In some embodiments, the tumor is hematological malignancy. In some embodiments, the tumor is cervical cancer, colon cancer, liver cancer, prostate cancer, melanoma, ovarian cancer, lung cancer, renal cell carcinoma, Schwannoma, mesothelioma, acute myeloid leukemia, multiple myeloma, non-Hodgkin lymphoma, or a combination thereof. In some embodiments, the tumor progression in the subject is reduced by at least 10%, at least 20%, at least 30%, at least 40%, at least 50%, at least 60%, at least 70%, at least 80%, at least 90%, at least 95%, at least 98%, or at least 99%, as compared to the subjects received no treatment. In some embodiments, the tumor progression in the subject is reduced by more than 10%, more than 20%, more than 30%, more than 40%, more than 50%, more than 60%, more than 70%, more than 80%, more than 90%, more than 95%, more than 98%, or more than 99%, as compared to the subjects received no treatment.

The methods disclosed herein may be used for treating or ameliorating a solid tumor or a hematological malignancy, for example, a cancer that is selected from the group consisting of breast cancer, cervical cancer, colon cancer, liver cancer, prostate cancer, melanoma, ovarian cancer, lung cancer, renal cell carcinoma, Schwannoma, mesothelioma, acute myeloid leukemia, multiple myeloma, non-Hodgkin lymphoma, and a combination thereof.

In some embodiments, the mutant SerRS protein has a decreased level of phosphorylation by ataxia telangiectasia mutated kinase (ATM), ataxia telangiectasia and Rad3-related kinase (ATR), or both.

Any of the phosphorylation-deficient SerRS proteins disclosed herein can be used in the methods and compositions for reducing tumor progression. For example, the phosphorylation-deficient variant SerRS protein can comprise an amino acid substitution at one or more of residues T22, S79 (or T79), S86, S101 (or T101), S142 (or T142), S217, S241, S255, S258, S262, S368, S394, S396, T214, T501, Y220 (or T220), Y248, and Y263 relative to the corresponding parent SerRS protein, or the corresponding wildtype SerRS protein. In some embodiments, the phosphorylation-deficient variant SerRS protein comprises an amino acid substitution at one or more of residues T22, S79, S86, S101, S142, S217, S241, S255, S258, S262, S368, S394, S396; T214, T501, Y220, Y248, and Y263 relative to human wildtype SerRS protein (for example, the SerRS protein having the sequence of SEQ ID NO: 1). In some embodiments, the amino acid substitution is one or more of serine-to-alanine, serine-to-glycine, serine-to-lysine, serine-to-arginine, serine-to-asparagine, serine-to-glutamine, serine-to-histidine, serine-to-cysteine, serine-to-valine, serine-to-leucine, serine-to-isoleucine, serine-to-proline, serine-to-methionine, serine-to-tryptophan, serine-to-phenylalanine, threonine-to-alanine, threonine-to-glycine, threonine-to-lysine, threonine-to-arginine, threonine-to-asparagine, threonine-to-glutamine, threonine-to-histidine, threonine-to-cysteine, threonine-to-valine, threonine-to-leucine, threonine-to-isoleucine, threonine-to-proline, threonine-to-methionine, threonine-to-tryptophan, threonine-to-phenylalanine, tyrosine-to-alanine, tyrosine-to-glycine, tyrosine-to-lysine, tyrosine-to-arginine, tyrosine-to-asparagine, tyrosine-to-glutamine, tyrosine-to-histidine, tyrosine-to-cysteine, tyrosine-to-valine, tyrosine-to-leucine, tyrosine-to-isoleucine, tyrosine-to-proline, tyrosine-to-methionine, tyrosine-to-tryptophan, and tyrosine-to-phenylalanine. In some embodiments, the amino acid substitution is at one or more of residues S101 and S241. In some embodiments, the phosphorylation-deficient variant SerRS protein can comprise an amino acid substitution S101A, S241A, or both relative to the corresponding parent SerRS protein. In some embodiments, the phosphorylation-deficient variant SerRS protein can comprise an amino acid substitution S101A, S241A, or both relative to human wildtype SerRS protein (for example, the SerRS protein having the sequence of SEQ ID NO: 1). In some embodiments, the amino acid substitution is one or more of serine-to-alanine, serine-to-glycine, serine-to-lysine, serine-to-arginine, serine-to-asparagine, serine-to-glutamine, serine-to-histidine, serine-to-cysteine, serine-to-valine, serine-to-leucine, serine-to-isoleucine, serine-to-proline, serine-to-methionine, serine-to-tryptophan, serine-to-phenylalanine.

In some embodiments, the phosphorylation-deficient variant SerRS protein can comprise an amino acid deletion at one or more of residues T22, 579 (or T79), S86, S101 (or T101), S142 (or T142), S217, S241, S255, S258, S262, S368, S394, S396, T214, T501, Y220 (or T220), Y248, and Y263 relative to the corresponding parent SerRS protein or the corresponding wildtype SerRS protein. That is, in these embodiments, one or more amino acid residues T22, S79 (or T79), S86, S101 (or T101), S142 (or T142), S217, S241, S255, S258, S262, S368, S394, 5396, T214, T501, Y220 (or T220), Y248, and Y263 in the corresponding parent SerRS protein can be absent in the phosphorylation-deficient variant SerRS protein. In some embodiments, the phosphorylation-deficient SerRS protein comprises an amino acid deletion at one or more of residues T22, S79, S86, S101, S142, S217, S241, S255, S258, S262, S368, S394, S396; T214, T501; Y220, Y248, Y263 relative to human wildtype SerRS protein (for example, the SerRS protein having the sequence of SEQ 1D NO: 1). In some embodiments, the amino acid deletion is at one or more of residues S101 and S241.

In some embodiments, the phosphorylation-deficient variant SerRS protein at least one amino acid deletion and at least one amino acid substitution at residues 122, S79 (or T79), S86, S101 (or T101), S142 (or T142), S217, S241, S255, S258, S262, S368, S394, S396; T214, T501, Y220 (or T220), Y248, and Y263 relative to the corresponding parent SerRS protein or the corresponding wildtype SerRS protein. In some embodiments, the phosphorylation-deficient variant SerRS protein at least one amino acid deletion and at least one amino acid substitution at residues T22, 579, S86, S101, S142, S217, S241, S255, S258, S262, S368, S394, S396; T214, T501, Y220, Y248, and Y263 relative to the human wildtype SerRS protein.

In some embodiments, the phosphorylation-deficient variant SerRS protein comprises an amino acid sequence having at least 90% identity to the amino acid sequence set forth in SEQ ID NO:1 and comprise an amino acid deletion at one or both of residues S101 and S241 of SEQ ID NO: 1. In some embodiments, the phosphorylation-deficient variant SerRS protein comprises an amino acid sequence having at least 70%, at least 75%, at least 85%, at least 90%, at least 95%, at least 98%, or more sequence identity to the amino acid sequence set forth in SEQ ID NO: 1 and comprises an amino acid substitution at one or both of residues 5101 and S241 of SEQ ID NO: 1. The amino acid substitution can be, for example, serine-to-alanine, serine-to-glycine, serine-to-lysine, serine-to-arginine, serine-to-asparagine, serine-to-glutamine, serine-to-histidine, serine-to-cysteine, serine-to-valine, serine-to-leucine, serine-to-isoleucine, serine-to-proline, serine-to-methionine, serine-to-tryptophan, serine-to-phenylalanine, or a combination thereof.

In some embodiments, the phosphorylation-deficient variant SerRS protein comprises an amino acid sequence having at least 90% identity to the amino acid sequence set forth in SEQ ID NO: 1 and comprises an amino acid substitution at one or both of residues S101 and S241 of SEQ ID NO: 1, wherein the amino acid substitution is serine-to-alanine or serine-to-glycine. In some embodiments, the phosphorylation-deficient variant SerRS protein comprises, or consists of, an amino acid sequence having at least 70%, at least 75%, at least 85%, at least 90%, at least 95%, at least 98%, or more sequence identity to SEQ ID NO: 2, SEQ ID NO: 3 or SEQ ID NO: 4. In some embodiments, the phosphorylation-deficient variant SerRS protein is a vertebrate SerRS protein (for example, a human variant SerRS protein).

### Methods and Compositions for Regulating Angiogenesis

Methods and compositions for regulating angiogenesis are provided herein. The methods and compositions can be used in, for example, subjects that are suffering from or are at the risk of developing one or more angiogenesis-related diseases. Examples of angiogenesis-related diseases include, but are not limited to, cancer, arthritis, skin disorders (for example, skin aging, sunburns, wound healing, psoriasis, eczema, hemangioma, angiofibroma and Kaposi's sarcoma), eye diseases (for example, diabetic retinopathy, retrolental fibroplasia, macular degeneration, corneal vascularization, and neovascular glaucoma), and cardiovascular diseases.

### Methods and Compositions for Promoting Angiogenesis

In some embodiments, the methods and compositions are for promoting angiogenesis. For example, the method of promoting angiogenesis in a subject can comprise: administering a composition comprising a mutant SerRS protein to a subject in need, wherein the mutant SerRS protein is deficiency in repressing VEGF transcription as compared to the corresponding wildtype SerRS protein, or is effective in stimulating VEGF transcription. In some embodiments, the method of promoting angiogenesis in a subject can comprise: administering a composition comprising a mutant SerRS protein to a subject in need, wherein the mutant SerRS protein is deficiency in repressing VEGF transcription as compared to human wildtype SerRS protein (e.g. the SerRS protein having the sequence of SEQ ID NO: 1), whereby angiogenesis is promoted in the subject. In some embodiments, the method of promoting angiogenesis in a subject can comprise: administering a composition comprising a mutant SerRS protein to a subject in need, wherein the mutant SerRS protein stimulates VEGF transcription, whereby angiogenesis is promoted in the subject. The composition can be, for example, a pharmaceutical composition. The methods and compositions can be used in, for example, a subject suffers from one or more diseases or disorders that involve poor vascularisation or abnormal vasculature. In some embodiments, the subject suffers from or is at the risk of developing one or more of ischemic heart disease, cardiovascular diseases, and neurological diseases.

In some embodiments, the repression of VEGF transcription by the mutant SerRS protein is less than 70%, less than 60%, less than 50%, less than 40%, less than 30%, less than 20%, less than 10%, less than 5%, less than 3%, or less than 1% of the repression of VEGF transcription by the corresponding parent SerRS protein. In some embodiments, the repression of VEGF transcription by the mutant SerRS protein is, or is about, 70%, 60%, 50%, 40%, 30%, 20%, 10%, 5%, 3%. 1%, or a range between any two of these values, of the repression of VEGF transcription by the corresponding parent SerRS protein. In some embodiments, the repression of VEGF transcription by the mutant SerRS protein is less than 70%, less than 60%, less than 50%, less than 40%, less than 30%, less than 20%, less than 10%, less than 5%, less than 3%, or less than 1% of the repression of VEGF transcription by the corresponding wildtype SerRS protein. In some embodiments, the repression of VEGF transcription by the mutant SerRS protein is, or is about, 70%, 60%, 50%, 40%, 30%, 20%, 10%, 5%, 3%, 1%, or a range between any two of these values, of the repression of VEGF transcription by the corresponding wildtype SerRS protein. In some embodiments, the repression of VEGF transcription by the mutant SerRS protein is less than 70%, less than 60%, less than 50%, less than 40%, less than 30%, less than 20%, less than 10%, less than 5%, less than 3%, or less than 1% of the repression of VEGF transcription by human wildtype SerRS protein (for example, the SerRS protein having the sequence of SEQ ID NO: 1). In some embodiments, the repression of VEGF transcription by the mutant SerRS protein is, or is about, 70%, 60%, 50%, 40%, 30%. 20%, 10%, 5%, 3%, 1%, or a range between any two of these values, of the repression of VEGF transcription by human wildtype SerRS protein (e.g., the SerRS protein having the sequence of SEQ ID NO: 1). In some embodiments, the mutant SerRS protein does not repress VEGF transcription. In some embodiments, the mutant SerRS protein stimulates VEGF transcription.

Any of the variant SerRS proteins disclosed herein that is deficient in repressing VEGF transcription or any of the variant SerRS protein disclosed herein that can stimulate VEGF transcription can be used in the methods and compositions for promoting angiogenesis. In some embodiments, the variant SerRS protein comprise an amino acid substitution at one or more of residue position 22, 79, 86, 101, 142, 217, 241, 255, 258, 262, 368, 394, 396, 214, 501, 220, 248, and 263 corresponding to the relative parent SerRS protein or a wildtype SerRS protein (e.g., a human wildtype SerRS protein). For example, the variant SerRS protein comprise an amino acid substitution at one or more of residue corresponding to T22, S79, S86, S101, S142, S217, S241, S255, S258, S262, S368, S394, S396, T214, T501, Y220, Y248, and Y263 of human wildtype SerRS protein. In some embodiments, the variant SerRS protein can comprise an amino acid substitution at one or more of residues T22, S79 (or T79), S86, S101 (or T101), S142 (or T142), S217, S241, S255, S258, S262, S368, S394, S396; T214, T501, Y220 (or T220), Y248, and Y263 relative to the corresponding wild type SerRS protein. In some embodiments, the variant SerRS protein can comprise an amino acid substitution at one or more of residues T22, S79 (or T79), S86, S101 (or T101), S142 (or T142), 5217, S241, S255, S258, S262, S368, S394, S396; T214, T501, Y220 (or T220), Y248, and Y263 relative to the corresponding parent SerRS protein. In some embodiments, the variant SerRS protein can comprise an amino acid substitution at one or more of residues T22, S79, S86, S101, S142, S217, S241, S255, S258, S262, S368, S394, S396; T214, T501; Y220, Y248, and Y263 relative to human wild type SerRS protein (for example, the SerRS protein having the sequence of SEQ ID NO: 1). Non-limiting examples of the amino acid substitutions comprises serine-to-aspartic acid, serine-to-glutamic acid, threonine-to-aspartic acid, and threonine-to-glutamic acid. In some embodiments, the mutant SerRS protein comprises an amino acid substitution at S101 (or T101), S241, or both relative to the corresponding wildtype SerRS protein or the corresponding parent SerRS protein. In some embodiments, the mutant SerRS protein comprises an amino acid substitution at S101, S241, or both relative to human wildtype SerRS protein (for example, the SerRS protein having the sequence of SEQ ID NO: 1). In some embodiments, the mutant SerRS protein comprises an amino acid substitution S101D (or T101 D), S241D, or both relative to the corresponding wildtype SerRS protein. In some embodiments, the mutant SerRS protein comprises an amino acid substitution S101D, S241D, or both relative to human wildtype SerRS protein (for example, the SerRS protein having the sequence of SEQ ID NO: 1). The mutant SerRS protein can be, for example, a vertebrate protein (e.g., a mammalian protein (including but not limited to a mutant human protein)), a chimeric SerRS protein, or a variant of a parent SerRS having a consensus SerRS sequence.

In some embodiments, the variant SerRS proteins that is deficient in repressing VEGF transcription comprises an amino acid sequence having at least 80%, at least 85%, at least 90%, at least 95%, at least 98%, at least 99%, or more sequence identity to the amino acid sequence set forth in SEQ ID NO: 1 and comprises an amino acid substitution on one or both of residues S101 and S241 in SEQ ID NO: 1, wherein the amino acid substitution serine-to-aspartic acid or serine-to-glutamic acid. In some embodiments, the variant SerRS protein that is deficient in repressing VEGF transcription comprises, or consists of, an amino acid sequence that has at least 80%, at least 85%, at least 90%, at least 95%, at least 98%, at least 99% sequence identity to SEQ ID NO: 5 or SEQ ID NO: 6. In some embodiments, the variant SerRS proteins that can stimulate VEGF transcription comprises an amino acid sequence having at least 80%, at least 85%, at least 90%, at least 95%, at least 98%, at least 99%, or more sequence identity to the amino acid sequence set forth in SEQ ID NO: 1 and comprises an amino acid substitution on one or both of residues S101 and S241 in SEQ ID NO: 1, wherein the amino acid substitution serine-to-aspartic acid or serine-to-glutamic acid. In some embodiments, the variant SerRS protein that can stimulate VEGF transcription comprises, or consists of, an amino acid sequence that has at least 80%, at least 85%, at least 90%, at least 95%, at least 98%, at least 99% sequence identity to SEQ ID NO: 5 or SEQ ID NO: 6.

### Methods and Compositions for Reducing Angiogenesis

The methods and compositions for reducing angiogenesis are disclosed herein. For example, the method of reducing angiogenesis in a subject can comprise: administering a composition comprising a variant SerRS protein (e.g., a mutant SerRS protein) to a subject in need, wherein the variant SerRS protein is a phosphorylation-deficient, whereby angiogenesis is reduced in the subject. In some embodiments, the maximum and/or average level of phosphorylation of the variant SerRS protein is less than 50%, less than 40%, less than 30%, less than 20%, less than 10%, less than 5%, less than 3%, less than 1%, of that of the corresponding wildtype SerRS protein or that of the parent SerRS protein. In some embodiments, the maximum and/or average level of phosphorylation of the variant SerRS protein is less than 50%, less than 40%, less than 30%, less than 20%, less than 10%, less than 5%, less than 3%, less than 1%, of that of human wildtype SerRS protein (for example, the SerRS protein having the sequence of SEQ ID NO: 1).

The composition can be, for example, a pharmaceutical composition. In some embodiments, angiogenesis is reduced by 10%, 20%, 30%, 40%, 50%, 60%, 70%, 80%, 90%, 95%, 98%, 99%, or more as compared to the subjects received no treatment. Without being bound by any particular theory, it is believed that phosphorylated SerRS proteins can repress transcription of vascular endothelial growth factor (VEGF) in the subject, which can lead to a reduction in angiogenesis. In some embodiments, the reduction in angiogenesis can lead to reduction in tumor progression in a subject having tumor. Angiogenesis in the subject may be reduced, for example, by 10%, 20%, 30%, 40%, 50%, 60%, 70%, 80%, 90%, 95%, 98%, 99%, or a range between any two of these values, as compared to the subjects received no treatment. In some embodiments, the angiogenesis is hypoxia-induced angiogenesis. In some embodiments, angiogenesis in the subject is reduced by more than 10%, more than 20%, more than 30%, more than 40%, more than 50%, more than 60%, more than 70%, more than 80%, more than 90%, more than 95%, more than 98%, or more than 99%, as compared to the subjects received no treatment. In some embodiments, angiogenesis in the subject is reduced by at least 10%, at least 20%, at least 30%, at least 40%, at least 50%, at least 60%, at least 70%, at least 80%, at least 90%, at least 95%, at least 98%, or at least 99%, as compared to the subjects received no treatment.

In some embodiments, the mutant SerRS protein has a decreased level of phosphorylation by ataxia telangiectasia mutated kinase (ATM), ataxia telangiectasia and Rad3-related kinase (ATR), or both.

Any of the phosphorylation-deficient SerRS proteins disclosed herein can be used in the methods and compositions for reducing angiogenesis. In some embodiments, the variant SerRS protein comprise an amino acid substitution at one or more of residue position 22, 79, 86, 101, 142, 217, 241, 255, 258, 262, 368, 394, 396, 214, 501, 220, 248, and 263 corresponding to the relative parent SerRS protein or a wildtype SerRS protein (e.g., a human wildtype SerRS protein). For example, the variant SerRS protein comprise an amino acid substitution at one or more of residue corresponding to T22, S79, S86, S101, S142, S217, S241, S255, S258, S262, S368, S394, S396, T214, T501, Y220, Y248, and Y263 of human wildtype SerRS protein. In some embodiments, the phosphorylation-deficient variant SerRS protein can comprise an amino acid substitution at one or more of residues T22, S79 (or T79), S86, S101 (or TI 01), S142 (or T142), S217, S241, S255, S258, S262, S368, S394, S396, T214, T501, Y220 (or T220), Y248, and Y263 relative to the corresponding wildtype SerRS protein or the parent SerRS protein. In some embodiments, the phosphorylation-deficient variant SerRS protein comprises an amino acid substitution at one or more of residues T22, S79, S86, S101, S142, S217, S241, S255, S258, S262, S368, S394, S396; T214, T501, Y220, Y248, and Y263 relative to human wildtype SerRS protein (for example, SerRS protein having the sequence of SEQ ID NO: 1). Examples of the amino acid substitutions include, but are not limited to serine-to-alanine, serine-to-glycine, serine-to-lysine, serine-to-arginine, serine-to-asparagine, serine-to-glutamine, serine-to-histidine, serine-to-cysteine, serine-to-valine, serine-to-leucine, serine-to-isoleucine, serine-to-proline, serine-to-methionine, serine-to-tryptophan, serine-to-phenylalanine, threonine-to-alanine, threonine-to-glycine, threonine-to-lysine, threonine-to-arginine, threonine-to-asparagine, threonine-to-glutamine, threonine-to-histidine, threonine-to-cysteine, threonine-to-valine, threonine-to-leucine, threonine-to-isoleucine, threonine-to-proline, threonine-to-methionine, threonine-to-tryptophan, threonine-to-phenylalanine, tyrosine-to-alanine, tyrosine-to-glycine, tyrosine-to-lysine, tyrosine-to-arginine, tyrosine-to-asparagine, tyrosine-to-glutamine, tyrosine-to-histidine, tyrosine-to-cysteine, tyrosine-to-valine, tyrosine-to-leucine, tyrosine-to-isoleucine, tyrosine-to-proline, tyrosine-to-methionine, tyrosine-to-tryptophan, and tyrosine-to-phenylalanine. In some embodiments, the amino acid substitution is at one or more of residues S101 (or T101) and S241. In some embodiments, the phosphorylation-deficient variant SerRS protein can comprise an amino acid substitution S101A, S241A, or both relative to the corresponding wildtype SerRS protein or the parent SerRS protein. In some embodiments, the phosphorylation-deficient variant SerRS protein can comprise an amino acid substitution S101A, S241A, or both relative to human wildtype SerRS protein (for example, SerRS protein having the sequence of SEQ ID NO: 1). In some embodiments, the amino acid substitution is serine-to-alanine, serine-to-glycine, serine-to-lysine, serine-to-arginine, serine-to-asparagine, serine-to-glutamine, serine-to-histidine, serine-to-cysteine, serine-to-valine, serine-to-leucine, serine-to-isoleucine, serine-to-proline, serine-to-methionine, serine-to-tryptophan, serine-to-phenylalanine, or a combination thereof.

In some embodiments, the phosphorylation-deficient variant SerRS protein comprise an amino acid deletion at one or more of residue position 22, 79, 86, 101, 142, 217, 241, 255, 258, 262, 368, 394, 396, 214, 501, 220, 248, and 263 corresponding to the relative parent SerRS protein or a wildtype SerRS protein (e.g., a human wildtype SerRS protein). For example, the variant SerRS protein comprise an amino acid deletion at one or more of residue corresponding to T22, 579, S86, S101, S142, S217, S241, S255, S258, S262, S368, S394, S396, T214, T501, Y220, Y248, and Y263 of human wildtype SerRS protein. In some embodiments, the phosphorylation-deficient variant SerRS protein can comprise an amino acid deletion at one or more of residues T22, S79 (or T79), S86, S101 (or T101), S142 (or T142), S217, S241, S255, S258, S262, S368, S394, S396, T214, T501, Y220 (or T220), Y248, and Y263 relative to the corresponding wildtype SerRS protein or the parent SerRS protein. In other words, one or more amino acid residues T22, S79 (or T79), S86, S101 (or T101), S142 (or T142), S217, S241, S255, S258, S262, S368, S394, S396, T214, T501, Y220 (or T220), Y248, and Y263 in the corresponding wildtype SerRS protein or the parent SerRS protein are absent in the phosphorylation-deficient variant SerRS protein. In some embodiments, the phosphorylation-deficient SerRS protein comprises an amino acid deletion at one or more of residues T22, S79, S86, S101, S142, S217, S241, S255, S258, S262, S368, S394, S396, T214, T501, Y220, Y248, and Y263 relative to human wildtype SerRS protein (for example, SerRS protein having the sequence of SEQ ID NO: 1). In some embodiments, the amino acid deletion is at one or more of residues S101 and S241.

In some embodiments, the phosphorylation-deficient variant SerRS protein comprise one or more amino acid deletions and one or more of amino acid substitutions at residue position 22, 79, 86, 101, 142, 217, 241, 255, 258, 262, 368, 394, 396, 214, 501, 220, 248, and 263 corresponding to the relative parent SerRS protein or a wildtype SerRS protein (e.g., a human wildtype SerRS protein). For example, the variant SerRS protein comprise one or more amino acid deletions and one or more of amino acid substitutions at residue corresponding to T22, S79, S86, S101, S142, S217, S241, S255, S258, S262, S368, S394, S396, T214, T501, Y220, Y248, and Y263 of human wildtype SerRS protein. In some embodiments, the phosphorylation-deficient variant SerRS protein comprises one or more amino acid deletions and one or more amino acid substitutions at residues T22, S79, S86, 5101, S142, S217, S241, S255, S258, S262, S368, S394, S396; T214, T501, Y220, Y248, and Y263 relative to the corresponding wildtype SerRS protein or the parent SerRS protein. In some embodiments, the phosphorylation-deficient variant SerRS protein comprises one or more amino acid deletions and one or more amino acid substitutions at residues T22, S79, S86, 5101, S142, S217, S241, S255, S258, S262, S368, S394, S396, T214, T501, Y220, Y248, and Y263 relative to human wildtype SerRS protein (for example, SerRS protein having the sequence of SEQ ID NO: 1).

In some embodiments, the phosphorylation-deficient variant SerRS protein comprises an amino acid sequence having at least 80%, at least 85%, at least 90%, at least 95%, at least 98%, at least 99%, or more sequence identity to the amino acid sequence set forth in SEQ ID NO: 11 and comprise an amino acid deletion at one or both of residues S101 and S241 of SEQ ID NO: 1. In some embodiments, the phosphorylation-deficient variant SerRS protein comprises an amino acid sequence having at least 80%, at least 85%, at least 90%, at least 95%, at least 98%, at least 99%, or more sequence identity to the amino acid sequence set forth in SEQ ID NO: 1 and comprises an amino acid substitution at one or both of residues S101 and S241 of SEQ ID NO: 1. In some embodiments, the amino acid substitution is selected from the group consisting of serine-to-alanine, serine-to-glycine, serine-to-lysine, serine-to-arginine, serine-to-asparagine, serine-to-glutamine, serine-to-histidine, serine-to-cysteine, serine-to-valine, serine-to-leucine, serine-to-isoleucine, serine-to-proline, serine-to-methionine, serine-to-tryptophan, and serine-to-phenylalanine.

In some embodiments, the phosphorylation-deficient variant SerRS protein comprises an amino acid sequence having at least 80%, at least 85%, at least 90%, at least 95%, at least 98%, at least 99%, or more sequence identity to the amino acid sequence set forth in SEQ ID NO: 1 and comprises an amino acid substitution at one or both of residues S101 and S241 of SEQ ID NO: 1, wherein the amino acid substitution is serine-to-alanine or serine-to-glycine. In some embodiments, the phosphorylation-deficient variant SerRS protein comprises, or consists of, an amino acid sequence having at least 80%, at least 85%, at least 90%, at least 95%, at least 98%, at least 99%, or more sequence identity to SEQ ID NO: 2, SEQ ID NO: 3, or SEQ ID NO: 4. In some embodiments, the phosphorylation-deficient variant SerRS protein is a vertebrate variant SerRS protein (for example, a mammalian variant SerRS protein (including and not limited to a human variant SerRS protein)).

In some embodiments, the method for reducing angiogenesis in a subject comprises: administering a composition comprising a SerRS phosphorylation inhibitor to a subject in need, whereby angiogenesis is reduced in the subject. The method can further comprise identifying a subject in need, wherein the subject is suffering from or is at the risk of developing a disease or disorder having abnormally high vascularization. The composition can be, in some embodiments, a pharmaceutical composition.

The term “SerRS phosphorylation inhibitor” is used herein in a broad sense and includes any molecule that partially or fully blocks, inhibits or neutralizes the phosphorylation of SerRS. In some embodiments, it can reduce, prevent, or abolish the phosphorylation of SerRS. The method/mechanism by which the phosphorylation of SerRS is inhibited is not limited in any way. In some embodiments, the SerRS phosphorylation inhibitor can act directly on SerRS, for example by binding to SerRS, to prevent or reduce phosphorylation of SerRS. In some embodiments, the SerRS phosphorylation inhibitor can act directly on a phosphorylase that can phosphorylate SerRS, for example by binding to the phosphorylase, to prevent or reduce phosphorylation of SerRS. In some embodiments, the SerRS phosphorylation inhibitor can interfere, preferably abolish or reduce, SerRS from interacting with a phosphorylase that can phosphorylate SerRS. In some embodiments, the SerRS phosphorylation inhibitor can modulate the expression level of a gene encoding a phosphorylase that can phosphorylate SerRS, for example, by inhibiting or reducing the transcription of the phosphorylase gene. In some embodiments, the SerRS phosphorylation inhibitor can modulate the levels the phosphorylase in cells by, for example, inhibiting or reducing the translation of the phosphorylase mRNA, or increasing the degradation of the phosphorylase mRNA or phosphorylase protein.

The types of SerRS phosphorylation inhibitors are not limited in any way. For example, the SerRS phosphorylation inhibitors can be small molecules, nucleic acids, antibodies, peptides, or any combination thereof. In some embodiments, the SerRS phosphorylation inhibitor can be a small molecule that binds to SerRS, a phosphorylase that phosphorylates SerRS, or both. In some embodiments, the SerRS phosphorylation inhibitor can be a molecule that blocks interaction of SerRS and one or more phosphorylases that phosphorylate SerRS. Non-limiting examples of the SerRS phosphorylation inhibitors include an inhibitor for ataxia telangiectasia mutated kinase (ATM), ataxia telangiectasia and Rad3-related kinase (ATR), or combinations thereof. In some embodiments, the SerRS phosphorylation inhibitor is an ATM inhibitor. In some embodiments, the SerRS phosphorylation inhibitor is an ATR inhibitor. In some embodiments, the SerRS phosphorylation inhibitor is a nucleic acid, for example, an anti-ATM small-hairpin RNA (shRNA), an ATM anti-sense RNA, an anti-ATR small-hairpin RNA (shRNA) or an ATR anti-sense RNA. In some embodiments, the SerRS phosphorylation inhibitor is ATR inhibitor VE-821. In some embodiments, the SerRS phosphorylation inhibitor is ATM inhibitor KU-55933.

The potential inhibitory or binding effect of a chemical compound on SerRS phosphorylation may be analyzed prior to its actual synthesis and testing by the use of computer modelling techniques. If the theoretical structure of the given compound suggests insufficient interaction and association between the phosphorylase and SerRS, synthesis and testing of the compound is obviated. However, if computer modelling indicates a strong interaction, the molecule may then be synthesized and tested for its ability to bind to SerRS and inhibit using a suitable assay. In this manner, synthesis of inoperative compounds may be avoided. An inhibitory or other binding compound of SerRS may be computationally evaluated and designed by means of a series of steps in which chemical entities or fragments are screened and selected for their ability to associate with the individual binding pockets or other areas of SerRS. One of skill in the art may use various methods to test chemical entities or fragments for their ability to associate with SerRS and more particularly with the phosphorylation sites of the SerRS. In some embodiments, known SerRS phosphorylation inhibitor, such as ATR inhibitor VE-821 and ATM inhibitor KU-55933, may be used as a starting point for designing compounds that inhibit SerRS phosphorylation.

### Pharmaceutical Compositions

Some embodiments disclosed herein provide pharmaceutical compositions comprising one or more variant SerRS proteins (for example, mutant SerRS proteins). In some embodiments, the variant SerRS protein is phosphorylation deficient. In some embodiments, the variant SerRS protein (for example, mutant SerRS proteins) is deficient in repressing VEGF transcription, for example, compared to the corresponding parent SerRS protein (for example, wildtype SerRS protein). Some embodiments disclosed herein provide pharmaceutical compositions comprising one or more SerRS phosphorylation inhibitors (for example, ATM inhibitors, ATR inhibitors, or both). The pharmaceutical composition can comprise one or more pharmaceutically acceptable excipient. The pharmaceutical composition can be used for the treatment of various disorders/diseases, including but not limited to, angiogenesis-related disorders/diseases, tumors, and cancers.

Also provided are pharmaceutically acceptable prodrugs of the pharmaceutical compositions, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the agent). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in Bundgaard. Design of Prodrugs (Elsevier Press, 1985).

Also provided are pharmaceutically active metabolites of the pharmaceutical compositions, and uses of such metabolites in the methods of the invention. A “pharmaceutically active metabolite” means a pharmacologically active product of metabolism in the body of a compound or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

Any suitable formulation of the compounds described herein can be prepared. See, generally, Remington's Pharmaceutical Sciences, (2000) Hoover, J. E. editor, 20th edition, Lippincott Williams and Wilkins Publishing Company, Easton, Pa., pages 780-857. A formulation is selected to be suitable for an appropriate route of administration. Some routes of administration are oral, parenteral, by inhalation, topical, rectal, nasal, buccal, vaginal, via an implanted reservoir, or other drug administration methods. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example, by a sufficiently basic compound such as an amine with a suitable acid, affording a physiologically acceptable anion. Alkali metal (e.g., sodium, potassium or lithium) or alkaline earth metal (e.g., calcium) salts of carboxylic acids also are made.

Where contemplated compounds are administered in a pharmacological composition, it is contemplated that the compounds can be formulated in admixture with a pharmaceutically acceptable excipient and/or carrier. For example, contemplated compounds can be administered orally as neutral compounds or as pharmaceutically acceptable salts, or intravenously in a physiological saline solution. Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

The pharmaceutical compositions as described herein are generally soluble in organic solvents such as chloroform, dichloromethane, ethyl acetate, ethanol, methanol, isopropanol, acetonitrile, glycerol, N,N-dimethylformamide, N,N-dimetheylaceatmide, dimethylsulfoxide, or any combination thereof. In one embodiment, the present invention provides formulations prepared by mixing an agent with a pharmaceutically acceptable carrier. In one aspect, the formulation may be prepared using a method comprising: a) dissolving a described agent in a water-soluble organic solvent, a non-ionic solvent, a water-soluble lipid, a cyclodextrin, a vitamin such as tocopherol, a fatty acid, a fatty acid ester, a phospholipid, or a combination thereof, to provide a solution; and b) adding saline or a buffer containing 1-10% carbohydrate solution. In one example, the carbohydrate comprises dextrose. The pharmaceutical compositions obtained using the present methods are stable and useful for animal and clinical applications.

Illustrative examples of water soluble organic solvents for use in the present methods include, but are not limited to, polyethylene glycol (PEG), alcohols, acetonitrile, N-methyl-2-pyrrolidone, N,N-dimethylformamide, N,N-dimethylacetamide, dimethyl sulfoxide, or a combination thereof. Examples of alcohols include but are not limited to methanol, ethanol, isopropanol, glycerol, or propylene glycol.

Illustrative examples of water soluble non-ionic surfactants for use in the present methods include, but are not limited to, CREMOPHOR® EL, polyethylene glycol modified CREMOPHOR® (polyoxyethyleneglyceroltriricinoleat 35), hydrogenated CREMOPHOR® RH40, hydrogenated CREMOPHOR® RH60, PEG-succinate, polysorbate 20, polysorbate 80, SOLUTOL HS (polyethylene glycol 660 12-hydroxystearate), sorbitan monooleate, poloxamer, LABRAFIL® (ethoxylated persic oil), LABRASOL® (capryl-caproyl macrogol-8-glyceride), GELUCIRE® (glycerol ester), SOFTIGEN® (PEG 6 caprylic glyceride), glycerin, glycol-polysorbate, or a combination thereof.

Illustrative examples of water soluble lipids for use in the present methods include, but are not limited to, vegetable oils, triglycerides, plant oils, or a combination thereof. Examples of lipid oils include, but are not limited to, castor oil, polyoxyl castor oil, corn oil, olive oil, cottonseed oil, peanut oil, peppermint oil, safflower oil, sesame oil, soybean oil, hydrogenated vegetable oil, hydrogenated soybean oil, a triglyceride of coconut oil, palm seed oil, and hydrogenated forms thereof, or a combination thereof.

Illustrative examples of fatty acids and fatty acid esters for use in the present methods include, but are not limited to, oleic acid, monoglycerides, diglycerides, a mono- or di-fatty acid ester of PEG, or a combination thereof.

Illustrative examples of cyclodextrins for use in the present methods include, but are not limited to, alpha-cyclodextrin, beta-cyclodextrin, hydroxypropyl-beta-cyclodextrin, or sulfobutyl ether-beta-cyclodextrin.

Illustrative examples of phospholipids for use in the present methods include, but are not limited to, soy phosphatidylcholine, or distearoyl phosphatidylglycerol, and hydrogenated forms thereof, or a combination thereof.

One of skill in the art may modify the formulations within the teachings of the specification to provide numerous formulations for a particular route of administration. For example, the compounds may be modified to render them more soluble in water or other vehicle. It is also well within the ordinary skill of the art to modify the route of administration and dosage regimen of a particular compound in order to manage the pharmacokinetics of the present compounds for maximum beneficial effect in a patient.

Pharmaceutical compositions disclosed herein, for example the compositions comprising a mutant SerRS protein that is phosphorylation-deficient, the compositions comprising a mutant SerRS protein that is deficient in repressing VEGF transcription, and the compositions comprising a mutant SerRS protein capable of stimulating VEGF transcription may be administered orally, parenterally, by inhalation, topically, rectally, nasally, buccally, vaginally, via an implanted reservoir, or other drug administration methods. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

A sterile injectable composition, such as a sterile injectable aqueous or oleaginous suspension, may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed include mannitol, water, Ringer's solution and isotonic sodium chloride solution. Suitable carriers and other pharmaceutical composition components are typically sterile.

In addition, sterile, fixed oils are conventionally employed as a solvent or suspending medium (e.g., synthetic mono- or diglycerides). Fatty acids, such as oleic acid and its glyceride derivatives, are useful in the preparation of injectables, as are pharmaceutically acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Various emulsifying agents or bioavailability enhancers which are commonly used in the manufacture of pharmaceutically acceptable solid, liquid, or other dosage forms can also be used for the purpose of formulation.

A composition for oral administration may be any orally acceptable dosage form including, but not limited to, tablets, capsules, emulsions and aqueous suspensions, dispersions and solutions. In the case of tablets for oral use, commonly used carriers include lactose and corn starch. Lubricating agents, such as magnesium stearate, can also be added. For oral administration in a capsule form, useful diluents include lactose and dried corn starch. When aqueous suspensions or emulsions are administered orally, the active ingredient can be suspended or dissolved in an oily phase combined with emulsifying or suspending agents. If needed, certain sweetening, flavoring, or coloring agents can be added. A nasal aerosol or inhalation compositions can be prepared according to techniques well-known in the art of pharmaceutical formulation and can be prepared as solutions in, for example saline, employing suitable preservatives (for example, benzyl alcohol), absorption promoters to enhance bioavailability, and/or other solubilizing or dispersing agents known in the art.

### EXAMPLES

Some aspects of the embodiments discussed above are disclosed in further detail in the following examples, which are not in any way intended to limit the scope of the present disclosure.

### Experimental Materials and Methods

The following experimental materials and methods were used for Examples 1-8 described below.

Cell Lines

HEK 293 cells, 3B11 cells, and MDA-MB-231 cells were purchased from American type culture collection (ATCC, Manassas, Va., USA) and cultured in Dulbecco's modified eagle medium (ThermoFisher Scientific, Grand Island, N.Y., USA) supplemented with heat inactivated fetal bovine serum (Omega Scientific, Tarzana, Calif., USA) to a final concentration of 10%. Transient transfections were performed using Lipofectamine 2000 (ThermoFisher Scientific). We established stable 3B11 cell lines and MDA-MB-231 cell lines expressing mouse or human SerRS mutants by pBabe-puro (Addgene, Cambridge, Mass., USA) retroviral infections and selection with puromycin (Sigma-Aldrich, St. Louis, Mo., USA). Hypoxic condition was achieved with a sealed hypoxia chamber (Stemcell Technologies, Vancouver, BC, Canada) in serum reduced (1%) medium.

Plasmid Constructs

Human and mouse full-length SerRS genes were cloned into the pFlag-CMV-2 vector (Sigma-Aldrich) and pBabe-puro vector (Addgene), and human SIRT2 genes into the pCDNA6-V5His-C vector (ThermoFisher Scientific). For mutations in SerRS, we performed site-directed mutagenesis PCR to obtain the SerRS^{S101A/S241A} and SerRS^{S101D/S241D} construct. The primer sequences for human SerRS mutant constructs are 5′ GAA AGT CGC ACA AAT CAA AAA AGT CCG ACT CCT CAT TG 3′ (SEQ ID NO: 7) and 5′ TGA TTT GTG CGA CTT TCA GGT TAG CTA AAG CGT C 3′ (SEQ ID NO: 8) for S101A: 5′ GAA AGT CGA CCA AAT CAA AAA AGT CCG ACT CCT CAT TG 3′ (SEQ ID NO: 9) and 5′ TGA TTT GGT CGA CTT TCA GGT TAG CTA AAG CGT C 3′ (SEQ ID NO: 10) for S101D; 5′ AGC TCG CAC AGT TTG ATG AAG AAC TTT ATA AGG 3′ (SEQ ID NO: 11) and 5′ AAC TGT GCG AGC TGT GCC ACC TCC TGC ATG ACC TCC 3′ (SEQ ID NO: 12) for S241A; 5′ AGC TCG ACC AGT TTG ATG AAG AAC TTT ATA AGG 3′ (SEQ ID NO: 13) and 5′ AAC TGG TCG AGC TGT GCC ACC TCC TGC ATG ACC TCC 3′ (SEQ ID NO: 14) for S241D. The primer sequences for mouse SerRS mutant constructs are 5′ GAA AGT CGC ACA GAT TAA AAA AGT CCG ACT CCT CAT TG 3′ (SEQ ID NO: 15) and 5′ TAA TCT GTG CGA CTT TCA GGG CAG CTA GCG CGT C 3′ (SEQ ID NO: 16) for S101A; 5′ GAA AGT CGA CCA GAT TAA AAA AGT CCG ACT CCT CAT TG 3′ (SEQ ID NO: 17) and 5′ TAA TCT GGT CGA CTT TCA GGG CAG CTA GCG CGT C 3′ (SEQ ID NO: 18) for S101D; 5′ CAG CTC GCC CAG TTT GAT GAA GAA CTT TAT AAG GTG 3′ (SEQ ID NO: 19) and 5′ CAA ACT GGG CGA GCT GGG CCA CTT CCT GCA TO 3′ (SEQ ID NO: 20) for S241A; 5′ CAG CTC GAC CAG TTT GAT GAA GAA CTT TAT AAG GTG 3′ (SEQ ID NO: 21) and 5′ CAA ACT GGT CGA GCT GGG CCA CTT CCT GCA TG 3′ (SEQ ID NO: 22) for S241D. The nucleotides shown in bold in the sequences of this paragraph encode the substituted residues.

For protein purification, human SerRS and its mutant genes were subeloned into pET-20b(+) plasmid (Novagen, Darmstadt, Germany) and overexpressed in E. coli. The recombinant C-terminal His6-tagged proteins were purified using Ni-NTA beads (Qiagen, Valencia, Calif. USA). The purities of the recombinant proteins were assessed by Coomassie blue staining following 4-12% Mini Gel (ThermoFisher Scientific) electrophoresis. Protein concentrations were determined using Bradford protein assay (BioRad, Hercules, Calif., USA).

RNAi 10134) DNA oligos encoding short-hairpin RNAs (shRNA) designed against human SerRS (5′ GGC ATA GGG ACC CAT CAT TGA 3′ (SEQ ID NO: 23) in 3′-UTR), GlyRS (5′ GCA TOG AGT ATC TCA CAA AGT 3′ (SEQ ID NO: 24) in the open reading frame), were inserted into the pLentiLox-hH1 plasmid, modified from the pLentiLox 3.7 plasmid to contain a H1 promoter (between Xba I and Xho I sites) to drive the shRNA expression. For non-targeting control shRNA, we used the sequence 5′ TAA GGC TAT GAA GAG ATA C 3′ (SEQ ID NO: 25). SiRNA duplexes against ATM and ATR were purchased from Cell Signaling Technology (Danvers, Mass., USA).

Real-Time PCR Assay

Total RNA was isolated from cells and zebrafish embryos by TRIzol Reagent (ThermoFisher Scientific). One gram of the total RNA from each sample was reversely transcribed to cDNA by M-MLV reverse transcriptase (Promega, Madison, Wis., USA). All real-time PCR reactions were performed using the StepOnePlus Real-Time PCR system (ThermoFisher Scientific) with SYBR Select Master Mix ThermoFisher Scientific). The primer pairs for the PCR reactions were: 5′ GAG GGC AGA ATC ATC ACG AAG 3′ (SEQ ID NO: 26) and 5′ TGT GCT GTA GGA AGC TCA TCT CTC 3′ (SEQ ID NO: 27) for human VEGFA; 5′ CGT CAC CAA CTG GGA CGA 3′ (SEQ ID NO: 28) and 5′ ATG GGG GAG GGC ATA CC 3′ (SEQ ID NO: 29) for human β-ACTIN; 5′ GGC TCT CCT CCA TCT GTC TGC 3′ (SEQ ID NO: 30) and 5′ CAG TGG TTT TCT TTC TTT CCT TTG 3′ (SEQ ID NO: 31) for zebrafish vegfa; 5′ TCA CCA CCA CAG CCG AAA GAG 3′ (SEQ ID NO: 32) and 5′ GTC AGC AAT GCC AG GTA CAT 3′ (SEQ ID NO: 33) for zebrafish β-actin. The PCR reaction program started at 95° C. for 10 min, followed by 45 cycles of 95° C. for 20 seconds and 60° C. for 1 min. Each experiment was carried out in triplicate. The VEGFA gene expression was normalized to that of β-ACTIN. Statistical analyses were performed with the software SigmaPlot (version 10.0). Student's t-test was used to analyze the changes between different groups.

In Vivo Studies in Zebrafish

Transgenic Tg (Fli1a: EGFP) fish were maintained as we described before (Ref). The fish embryos were kept at 28.5° C. before and after microinjection. The antisense morpholino (MOs) targeting SerRS was injected into the yolk of 1-cell stage embryos at the dosage of 4˜5 ng per embryo. The sequence of SerRS-MO is (Ref). The SerRS-MO (5′ AGG AGA ATG TGA ACA AAC CTG ACA C 3′ (SEQ ID NO: 34)) and standard control MO (5′ CCT CTT ACC TCA GTT ACA ATT TAT A 3′ (SEQ ID NO: 35)) were purchased from Gene Tools, LLC (Philomath, Oreg. USA). After injection, embryos were incubated in E3 embryo medium supplemented with 0.003% 1-phenyl-2-thiourea (PTU) at 28.5° C. to prevent pigment formation. Embryos were anesthetized with 0.168 mg mL⁻¹ tricaine (Sigma-Aldrich), mounted in 2% methylcellulose and photographed with a Nikon fluorescent microscope (AZ100) equipped with a Nikon CCD camera (Qimaging Retiga 2000R). All the experiments involving zebrafish had been conducted according to the guidelines established by the Institutional Animal Care and Use Committee (IACUC) at The Scripps Research Institute, IACUC approval number 09-0009. Statistical analyses were performed with the software SPSS Statistics 19. The rescue effects of different SerRS mutants on ISV development were analyzed with χ² test.

Immunoblotting and Immunoprecipitation

Cells were resuspended with lysis buffer (20 mM Tris-HCl (pH 7.5), 150 mM NaCl, 1 mM of EDTA, 1 mM EGTA, 1% Triton X-100, 2.5 mM sodium pyrophosphate, 1 mM beta-glycerophosphate, 1 mM Na₃VO₄, and protease inhibitor cocktail) on ice. Supernatants were incubated with indicated antibodies and protein-G-conjugated agarose beads (ThermoFisher Scientific) for at least 2 h. The beads were washed five times with wash buffer (same as the lysis buffer, except that Triton X-100 was reduced from 1% to 0.1%) and then subjected to SDS-PAGE and immunoblotting analysis with indicated antibodies. Protein samples from zebrafish were prepared using TRIzol Reagent (ThermoFisher Scientific). The monoclonal anti-Flag antibody for immunoprecipitation was purchased from Sigma-Aldrich. Custom-made rabbit anti-human SerRS antibody was raised against purified human recombinant SerRS and affinity-purified. Anti-ATM/ATR substrate p-SQ, anti-ATM, anti-p-ATM (serine 1981), anti-ATR, anti-SIRT2, anti-α-tubulin, anti-β-actin, anti-Lamin A/C, anti-P53, anti-p-P53 (serine 15), anti-RPA32, anti-p-RPA32 (serine 33), anti-CHK1, anti-p-CHK1 (serine 345), anti-CHK2, anti-p-CHK2 (threonine 68), and anti-HIF1β (ARNT) antibodies were purchased from Cell Signaling Technology. Anti-HIF1α antibody was purchased from Novus Biologicals (Littleton, Colo., USA). Anti-V5 and anti-GlyRS antibodies were purchased from ThermoFisher Scientific and Abnova (Walnut, Calif., USA), respectively.

Matrigel Plug Angiogenesis Assay

A total of 10⁶ of stably transfected 3B11 cells were resuspended in 100 μl of DMEM medium supplemented with 10% FBS and then mixed with 200 μl of ice-cold matrigel (BD Biosciences, San Jose, Calif., USA) liquid on ice. The 300 μl of cell and matrigel mixture was injected subcutaneously into a flank of C3H/HeJ mice (two injection sites per mouse and 5-6 mice in each group) (Jackson Laboratory). After inoculation for 14 days, the matrigel plugs were excised and frozen in Tissue-Tek® OCT compound for cryostat sectioning. All the mouse experiments were conducted according to the guidelines established by the Institutional Animal Care and Use Committee (IACUC) at The Scripps Research Institute, IACUC approval protocol number 13-0003.

Xenograft Tumor Model

100 of MDA-MB-231 cells stably transfected with vectors expressing wild type human SerRS, SerRS^{S101A/S241A}, or SerRS^{S101D/S241D} were subcutaneously injected into the mammal glands of 6-8-week-old female NOD.Cg-Prkdc^{scid}Il2rg^{tmJWjl}/SzJ mice (six mice in each group) (Jackson Laboratory). 14 days after injection, the tumor xenograft were separated from mice and frozen in Tissue-Tek® OCT compound for cryostat sectioning.

Immunohisiochemistry

5-μm sections from freshly frozen tumor xenograft and matrigel plugs were treated with acetone and 3% H₂O₂ to block endogenous peroxidase. After 3-5 times wash and goat serum block, the sections were incubated with an anti-CD31 antibody (1:3000; Cell Signaling Technology) overnight at 4° C. The blood vessels were counted in 5-10 random viable fields (120×) in tumor xenograft samples and microvessel density in matrigel plugs was quantified by measuring the CD31 staining density using Image J software. To detect the hypoxia, we incubated the slides with anti-HIF1α antibody (1:100; Novus Biologicals).

EMSA

The 27 bp DNA oligonucleotides corresponding to SerRS binding site on the VEGFA promoter (5′ GGC GGG GCG GAG CCA TGC GCC CCC CCC 3′ (SEQ ID NO: 36)) were synthesized, annealed, and [³²P]-labeled at the 5′ end by T4 DNA kinase (New England Biolabs, Ipswich, Mass., USA) before desalting using a sephadex G-25 spin column (GE Healthcare, Pittsburgh, Pa., USA). The labeled oligonucleotides (0.08 pmol) were incubated with recombinant SerRS at indicated concentrations in binding buffer (20 mM Tris-HCl, pH 8.0, 60 mM KCl, 5 mM MgCl₂, 0.1 mg ml⁻¹ BSA, 10 ng μl⁻¹ poly (dG-dC), 1 mM DTT) for 30 min at room temperature. The samples were loaded to 5% native polyacrylamide gel (17.5 cm in length) and underwent electrophoresis at 250 V in running buffer (25 mM Tris, pH 8.3, 190 mM glycine). Afterwards, the gel was dried and examined by autoradiography.

Cell Fractionation

The cytoplasmic and nuclear fractions were separated and extracted by using NE-PER® Nuclear and Cytoplasmic Extraction Kit (ThermoFisher Scientific). Exogenously expressed or endogenous SerRS proteins were detected by Western blot analysis using anti-flag polyclonal antibody (Sigma-Aldrich) or polyclonal anti-SerRS antibody.

Chromatin Immunoprecipitation (ChIP)

Cells were fixed with formaldehyde (1% final concentration) for 10 min at room temperature. The reaction was stopped by adding 125 mM of glycine. ChIP assays were performed according to the protocol of ChIP-IT Express Enzymatic kit (Active Motif) with affinity purified polyclonal anti-SerRS antibody. After 3 washes, ChIPed DNA was analyzed on the StepOnePlus Real-Time PCR system using SYBR Select Master Mix (Applied Biosystems). A primer set (5′-GGGCGGATGGGTAATTTTCA-3′ (SEQ ID NO: 37) and 5′-CTGCGGACGCCCAGTGAA-3′ (SEQ ID NO: 38)) targeting the VEGFA promoter was used.

### Example 1

### SerRS is Involved in the Hypoxia Response to Regulate VEGFA

This example shows that SerRS is involved in the hypoxia response to regulate VEGFA expression.

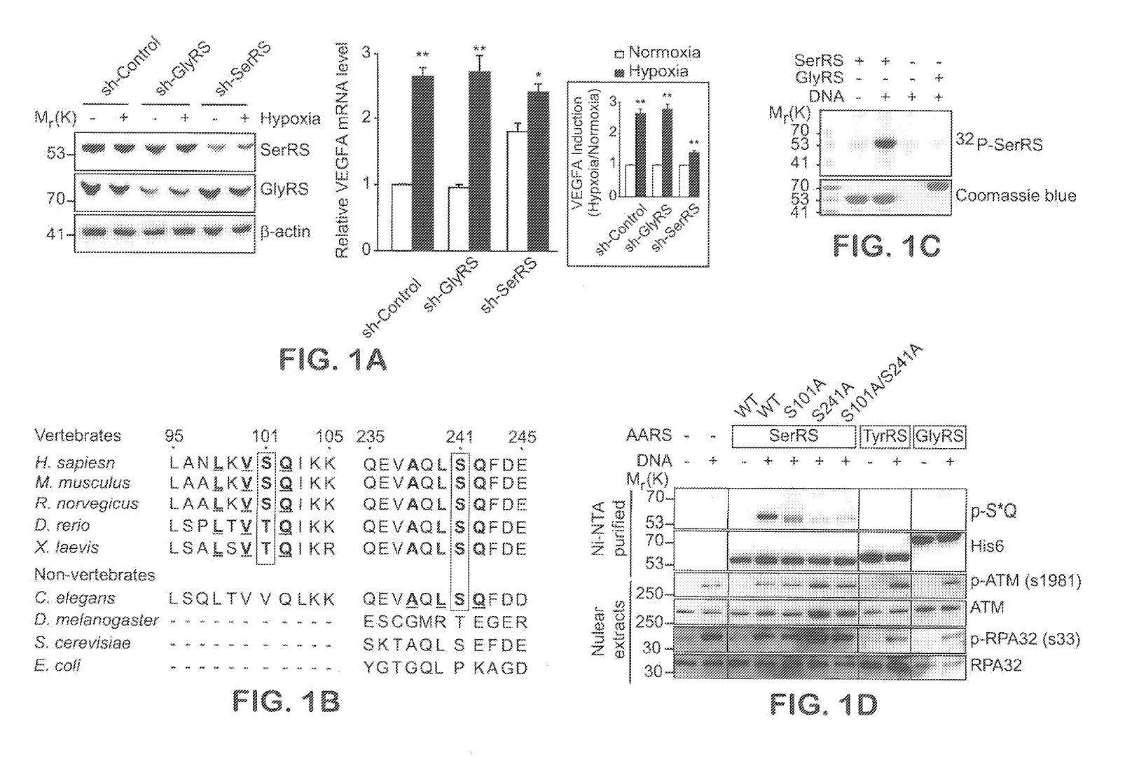

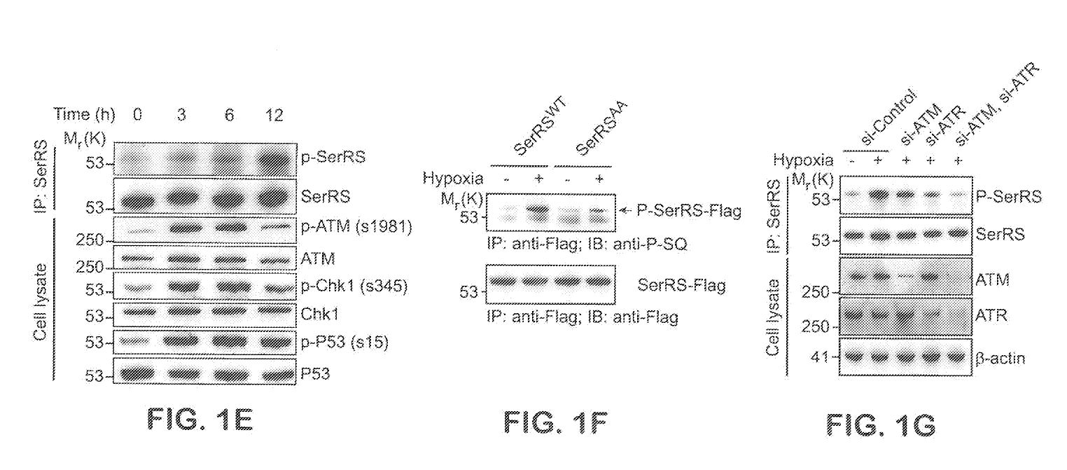

SerRS expression was knocked down in HEK293 cells with a short hairpin RNA (shRNA) targeting the 3′ untranslated region (3′-UTR) of the SerRS gene (FIG. 1A). At normal oxygen concentration (normoxia), as observed previously (Shi et al., 2014), VEGFA expression was upregulated upon knocking down of SerRS compared with control cells transfected with a nonspecific control shRNA (sh-control) or a shRNA targeting another aminoacyl-tRNA synthetase (sh-GlyRS) (FIG. 1A). However, under hypoxia, although VEGFA expression is significantly enhanced in control cells as expected, the hypoxia response in SerRS knocked down cells is greatly reduced (FIG. 1A and Inset), suggesting SerRS is involved in the hypoxia response to regulate VEGFA.

### Example 2

### SerRS is Involved in the Hypoxia Response to Regulate VEGFA

This example describe experiments testing whether the reduced VEGFA stimulation in SerRS knocked down cells is caused by inactivation of the role of SerRS in suppressing VEGFA by hypoxia.

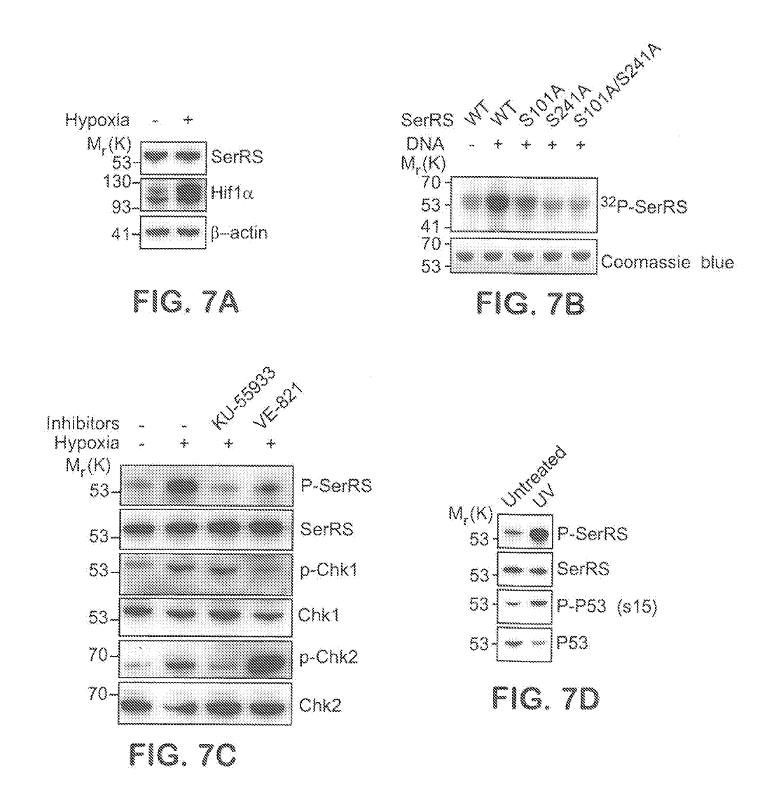

As shown in FIG. 7A, hypoxia does not affect the expression of SerRS. SerRS was investigated for potential posttranslational modifications. In a large-scale mass spectrometry study described in Matsuoka et al., 2007, SerRS was found to be phosphorylated at serine 241 (S241) by ATM/ATR kinases activated by DNA damage. In PhosphoSitePlus database (Hornbeck et al., 2015), another possible SerRS phosphorylation site serine 101 (S101) was also found. Both sites have the conserved ATM/ATR substrate motif with a serine or threonine followed by a glutamine and preceded by two hydrophobic residues (at −1 and −3 positions relative to the serine/threonine) (FIG. 1B). Multiple sequence alignment revealed strict conservation of S/T101 and S/T241 and flanking ATM/ATR substrate motif residues in vertebrate SerRS (FIG. 1B), concurrent with the role of SerRS in regulating vascular development and angiogenesis.

DNA fragment-induced SerRS phosphorylation was confirmed by ³²P-labelling in vitro. Double-stranded DNA oligonucleotides were added to the nuclear extract of HEK293 cells to mimic DNA damage to activate ATM/ATR. The “activated” nuclear extract specifically induced robust phosphorylation of the purified recombinant SerRS, but not GlyRS (FIG. 1C). SerRS phosphorylation was further verified by using specific phosphor-ATM/ATR substrate (p-SQ) antibody (FIG. 1D). To confirm the phosphorylation sites on SerRS, we substituted S101 and S241 with alanine separately to generate SerRS^{S101A} and SerRS^{S241A}) and simultaneously to generate SerRS^{S101A/S241A}, SerRS^{S101A} showed a decreased level of phosphorylation, whereas SerRS^{S241A} and SerRS^{S101A/S241A} almost had no phosphorylation in vitro as examined by both p-SQ antibody (FIG. 1D) and ³²P-labelling (FIG. 7B), suggesting that SerRS can be phosphorylated by ATM/ATR kinases at both S101 and S241, and that S241 is the major phosphorylation site on SerRS.

To confirm SerRS phosphorylation in cell, HEK293 cells were stimulated by stresses-including hypoxia and UV irradiation-that can activate ATM/ATR. Under hypoxia, phosphorylation of endogenous SerRS in HEK293 cells was detected within 12 hours (FIG. 1E). In hypoxic HEK293 cells, the exogenously expressed SerRS^{S101A/S241A} showed much weaker phosphorylation than wild type SerRS (SerRS^{WT}) (FIG. 1F), confirming that S241 and/or S101 are the major phosphorylation sites under hypoxia stress.

To further confirm that ATM and ATR are responsible for SerRS phosphorylation under hypoxia, ATM and ATR were knocked down either separately or simultaneously by siRNAs. Hypoxia-induced SerRS phosphorylation was greatly inhibited when either ATM or ATR was knocked down, and completely blocked when both kinases were knocked down simultaneously (FIG. 1G). Consistent with these results, SerRS phosphorylation under hypoxia could also be blocked by specific ATM and ATR inhibitor KU-55933 and VE-821, respectively (FIG. 7C). SerRS phosphorylation was also detected under UV irradiation (FIG. 7D).

### Example 3

### Phosphorylation Inactivates SerRS as a Transcriptional Repressor of VEGFA in Human Cells and in Zebrafish

This example shows that phosphorylation of SerRS leads to the loss of transcriptional repressor activity of SerRS.

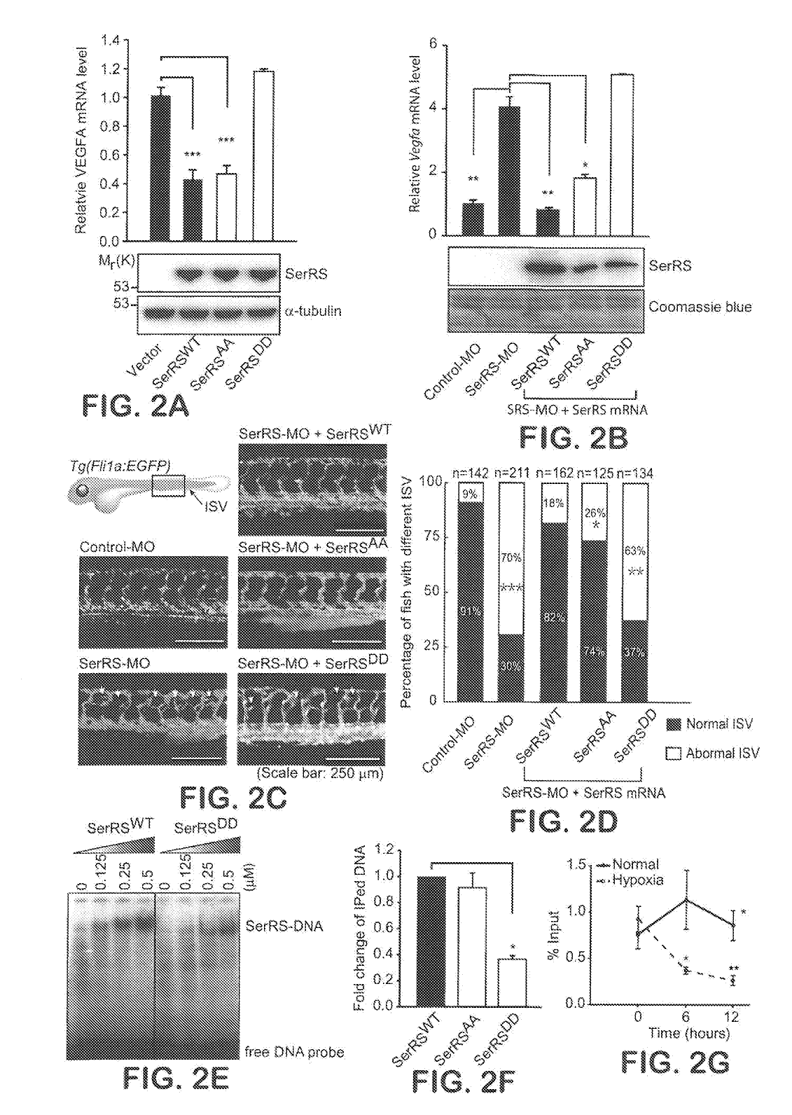

To understand if phosphorylation of SerRS affects its role as a transcriptional repressor of VEGFA, a mutant form of SerRS bearing double substitutions of S101 and S241 with aspartic acid residues (SerRS^{S101D/S241D} or SerRS^{S101D/S241D}) was generated to mimic the phosphorylated SerRS. In HEK293 cells, in contrast to SerRS^{WT} and SerRS^{S101A/S241A}, SerRS^{S101D/S241D} can no longer repress VEGFA transcription (FIG. 2A), suggesting that phosphorylation can completely inhibit the transcriptional repressor activity of SerRS.

To investigate the effect of SerRS phosphorylation in vivo, a previously established zebrafish system described in Fukui et al. 2009 and Xu et al. 2012 was used. In zebrafish embryos, endogenous SerRS expression was knocked down by an antisense morpholino (SerRS-MO), which led to a 4-fold increase in the mRNA level of Vegfa (FIG. 2B). This effect can be rescued or largely rescued by co-injection of human SerRS^{WT} mRNA or SerRS^{S101A/S241A} mRNA with SerRS-MO. However, as shown in FIG. 2B, co-injection of SerRS^{S101D/S241D} mRNA had no rescue effect at all, confirming that phosphorylation at S101 and S241 completely blocks the transcriptional repressor activity of SerRS in vivo.

### Example 4

### Phosphorylation Abolishes the Anti-Angiogenic Activity of SerRS in Zebrafish

In this example, the effect of SerRS phosphorylation on vascular development in zebrafish was examined.

FIGS. 2C and 2 show that knocking down SerRS by injecting SerRS-MO resulted in abnormal hyper-intersegmental vessel (ISV) branching phenotype in 69.7% (n=147 out of 211) of zebrafish embryos as expected. In contrast, only 9.2% (n=13 out of 142) of zebrafish embryos injected with a control morpholino (control-MO) exhibited the hyper-ISV phenotype. Co-injection of human SerRS^{S101A/S241A} mRNA largely rescued the abnormal ISV branching (26.4%, n=33 out of 125), which is comparable to the rescue effect of SerRS^{WT} mRNA (17.9%, n=29 out of 162) (FIGS. 2C and 2D). In contrast, SerRS^{S101D/S241D} could not rescue the abnormal ISV branching (62.7%, n=84 out of 134) (FIGS. 2C and 2D), confirming that SerRS phosphorylation blocks its anti-angiogenic activity in vivo.

### Example 5

### Phosphorylation Inactivates SerRS by Attenuating its DNA Binding Capacity

This example shows that phosphorylated SerRS has reduced DNA binding capacity.

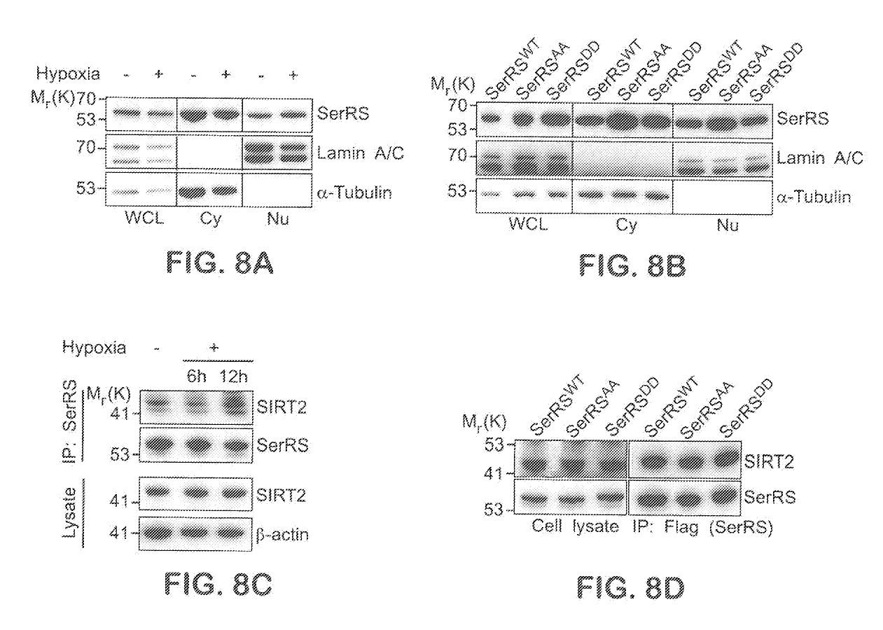

To explore the molecular mechanism of how SerRS phosphorylation inactivates its function as a transcriptional repressor, the effect of hypoxia on SerRS nuclear localization in HEK293 cells was examined. The result was negative (FIG. 8A). Consistently, similar cytoplasmic/nuclear distribution of exogenously expressed SerRS^{WT}, SerRS^{S101D/S241D}, and SerRS^{S101A/S241A} proteins was also found in HEK293 cells (FIG. 8B).

SIRT2 has been described in Shi et al. 2014 as a necessary co-factor for SerRS to epigenetically silence VEGFA expression. The interaction between SerRS and SIRT2 under hypoxia was examined. Similar amount of SIRT2 was co-immunoprecipitated with SerRS in HEK293 cells before and after the cells were cultured under hypoxia for 6 or 12 hours (FIG. 8C). Consistently, SIRT2 interacts with SerRS^{S101A/S241A} and SerRS^{S101D/S241D} as strong as with SerRS^{WT} (FIG. 8D), indicating that hypoxia does not affect the SerRS-SIRT2 interaction.

The effect of hypoxia on the interaction of SerRS with VEGFA promoter was also explored. As detected by electrophoresis mobility shift assay (EMSA), the direct binding between SerRS and a ³²P-labeled 27-bp DNA fragment previously identified as the SerRS binding site from the VEGFA promoter in Shi et al. 2014 was attenuated by the phosphor-mimicking mutant SerRS^{S101D/S241D} (FIG. 2E). In HEK293 cells, SerRS^{S101D/S241D} also showed a decreased binding on VEGFA promoter as determined by the chromatin immunoprecipitation assay (FIG. 2F). The assay was also performed with HEK293 cells during hypoxia, and showed a gradual decrease of the level of endogenous SerRS bound to the VEGFA promoter (FIG. 2G). These data show that hypoxia-induced phosphorylation blocks the transcriptional repressor activity of SerRS by weakening its DNA binding capacity.

### Example 6

### ATM/ATR-SerRS is a Key Pathway that Regulates Hypoxia-Induced Angiogenesis

This example describes the experiments studying to what extent the ATM/ATR-SerRS pathway contributes to the hypoxia-induced VEGFA expression.

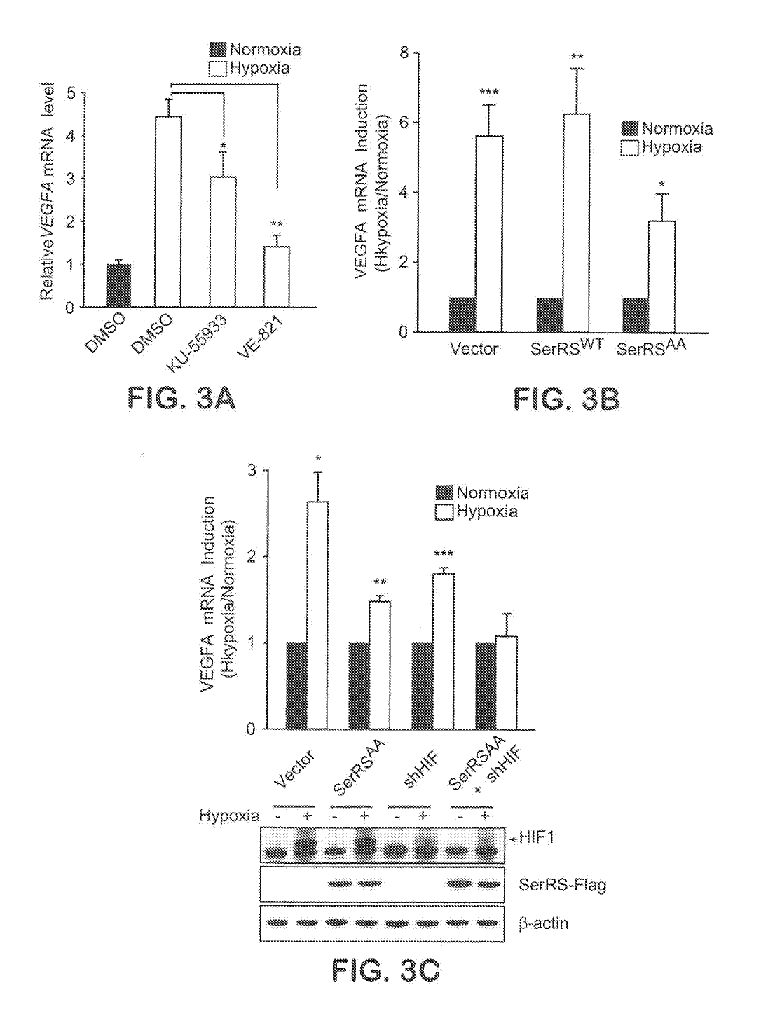

ATM or ATR was blocked in HEK293 cells by specific inhibitors. As shown in FIG. 3A, ATR inhibitor VE-821 dramatically inhibited VEGFA induction under hypoxia, whereas the effect of ATM inhibitor KU-55933 was less but still statistically significant, suggesting that ATM and ATR are important players in stimulating VEGFA expression during hypoxia.

ATM and ATR have many substrates, most of which are involved in DNA damage response. To test if SerRS is the main substrate that mediates the role of ATM/ATR to stimulate VEGFA expression under hypoxia, phosphorylation-deficient SerRS^{S101A/S241A} was introduced into HEK293 cells to block the ATM/ATR-SerRS pathway. Overexpression of SerRS^{S101A/S241A} significantly suppressed VEGFA induction, whereas overexpression of SerRS^{WT} had no effect (FIG. 3B). These results indicate that ATM/ATR-mediated SerRS phosphorylation to inactivate the transcriptional repressor of SerRS plays an important role in VEGFA induction under hypoxia.

### Example 7

### Blocking ATM/ATR-SerRS Pathway can Collaborate with HIF Knockdown to Achieve Complete Inhibition of Hypoxia-Induced VEGFA Expression

Although HIF is considered as the major hypoxia-induced transcriptional factor to promote VEGFA expression and angiogenesis, inhibiting HIF alone has not been able to completely block angiogenesis. Without being bound by any particular theory, it is believe that it is because of the involvement of HIF-independent pathways. See e.g., Lee and Lee, 2013, Mizukami et al., Mizukami et al. 2004. In view of the important role of the ATM/ATR-mediated SerRS phosphorylation in the hypoxia response, this example tests whether substantial or complete inhibition of VEGFA induction can be achieved by inhibiting HIF and, at the same time, blocking the ATM/ATR-SerRS pathway by expressing SerRS^{S101A/S241A}.

1 HEK293 cells were transfected with both shHIF-1α and shHIF-2α constructs together; however, HIF-2α was undetectable in the cells, consistent with its tissue specific expression pattern. As shown in FIG. 3C, knocking down HIF in HEK293 cells by shRNA (shHIF) could not completely block VEGFA induction by hypoxia. However, if we simultaneously express the constitutively active SerRS^{S101A/S241A} when HIF is knocked down, we completely inhibited VEGFA induction under hypoxia (FIG. 3C). This result not only demonstrated that the ATM/ATR-SerRS pathway is HIF-independent, but also suggested the potential of using SerRS^{S101A/S241A} in combination with HIF inhibition to achieve a complete suppression of hypoxia-induced angiogenesis. Further results showed that knockdown of HIF on top of overexpressing SerRS^{S101A/S241A} does not provide any additional efficacy, suggesting that SerRS^{S101A/S241A} can completely replace and overthrow the effect of HIF-1 inhibition.

### Example 8

### SerRS^{S101A/S241A} Bypasses Hypoxia Response and Strongly Inhibits Angiogenesis in Mice

This example describes experiments studying the activity of SerRS^{S101A/S241A} and the effect of SerRS phosphorylation in hypoxia-induced angiogenesis in mammal. It shows that on a murine model of triple negative human breast cancer, overexpression of a phosphorylation-deficient form of SerRS (SerRS^{S101A/S241A}) can suppress angiogenesis and tumor growth much more robustly than the knockdown of HIF-1, indicating that SerRS^{S101A/S241A} inhibits both HIF-dependent and HIF-independent hypoxia-response pathways

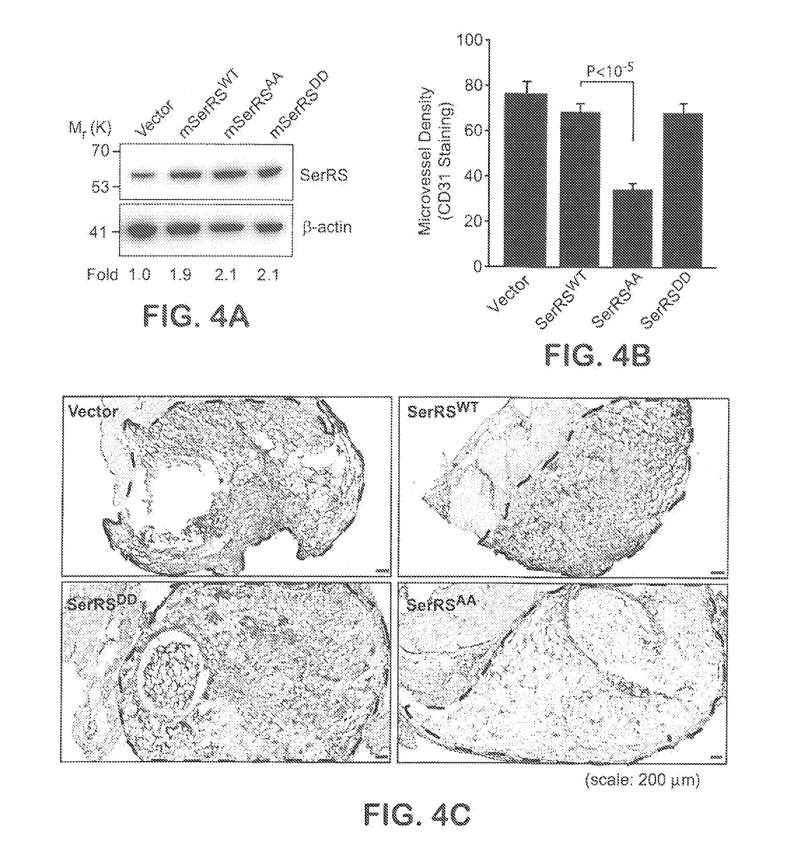

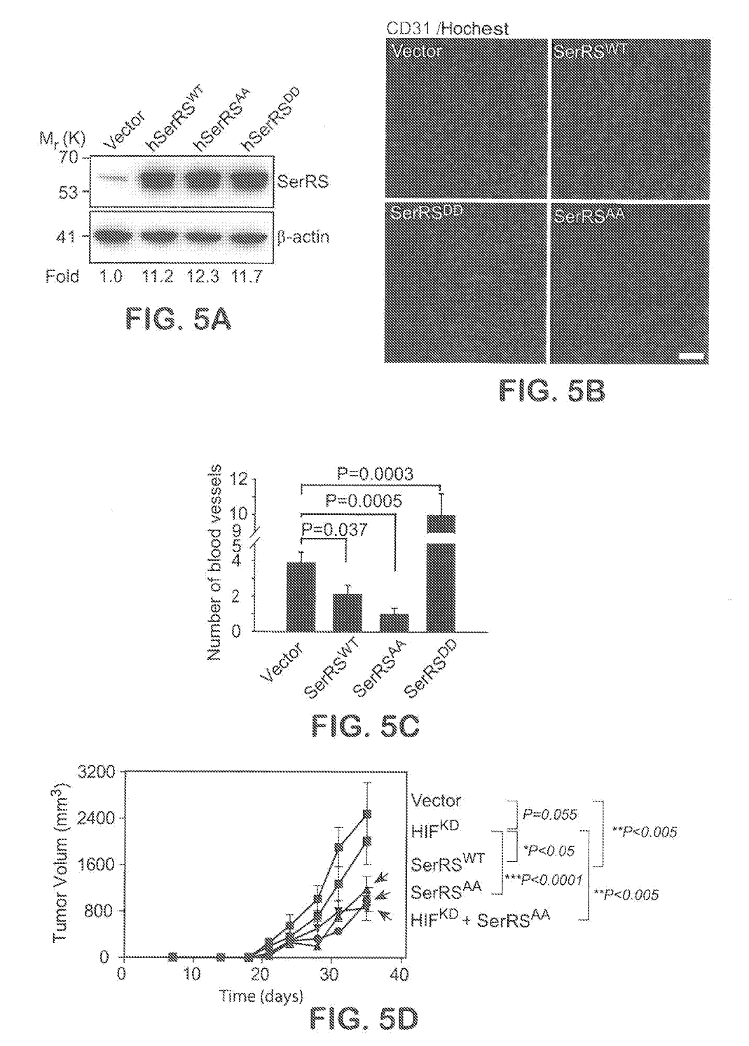

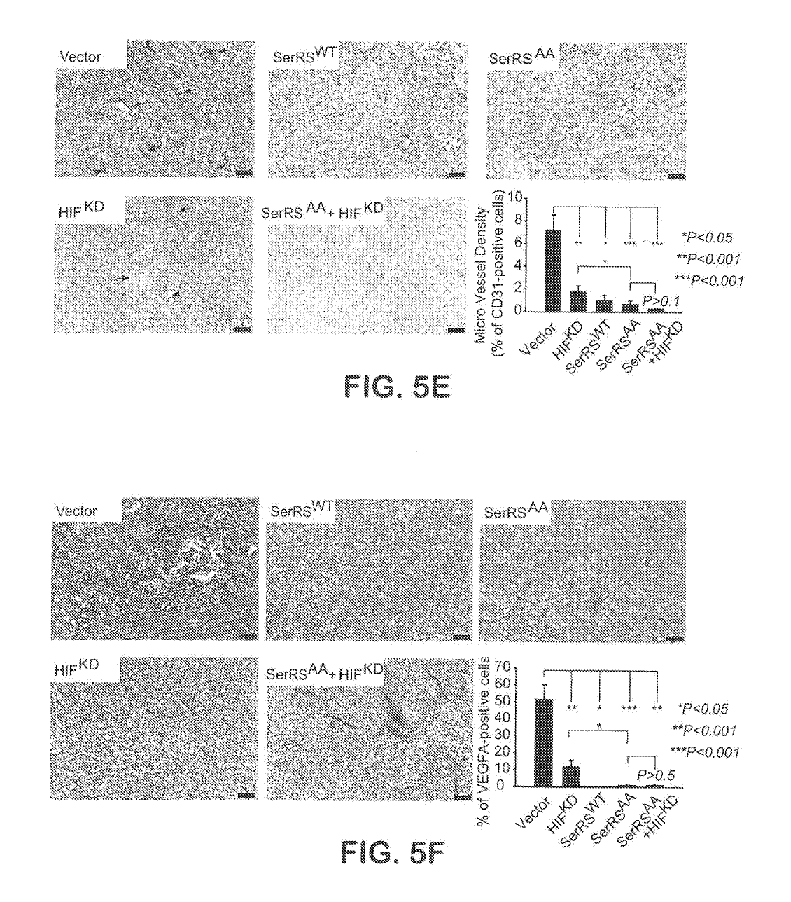

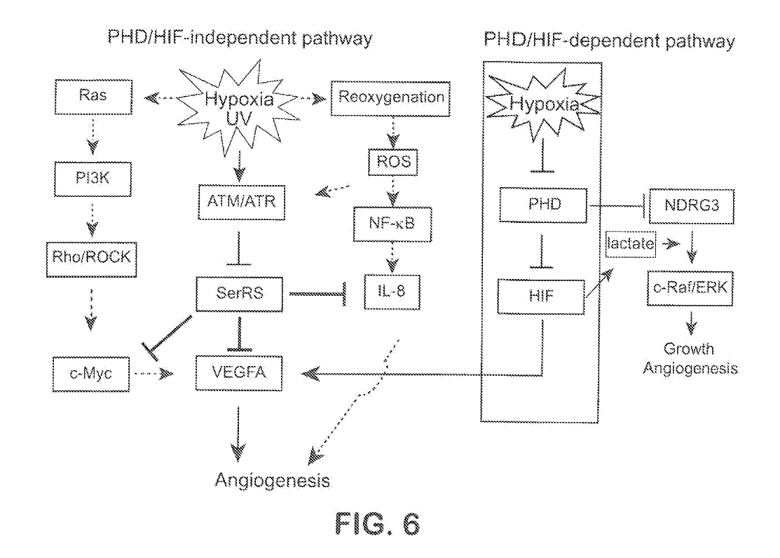

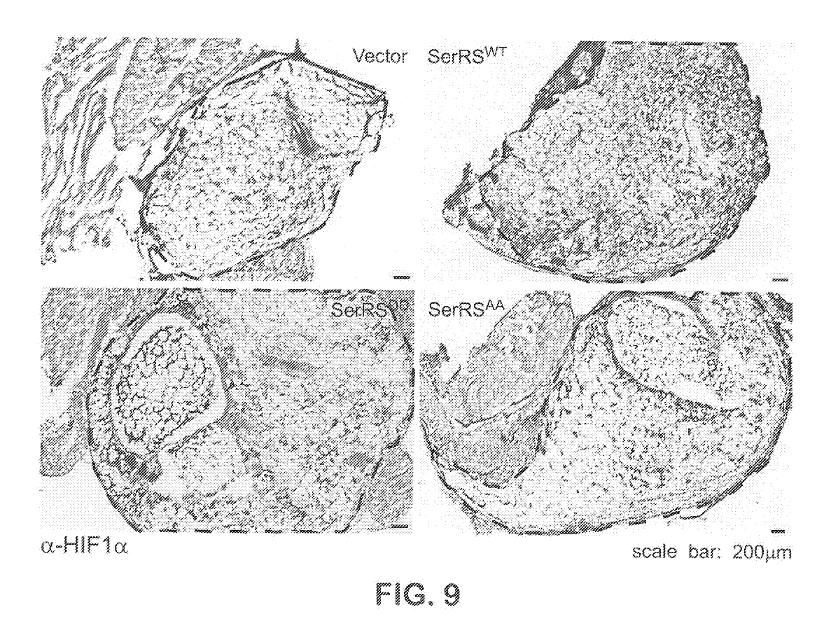

A matrigel plug angiogenesis assay was used. Mice endothelial 3B11 cells were stably transfected with mouse SerRS^{WT}, SerRS^{S101A/S241A}, or SerRS^{S101D/S241D} gene to achieve an expression level similar to that of the endogenous mouse SerRS (FIG. 4A). The engineered 3B11 cells were mixed with matrigel in vitro at low temperature. Each mixture was injected subcutaneously into mice to solidify into a plug, where a hypoxic environment would form prior to the induction of vasculature. Two weeks after the injection, by the elevated Hif-1α protein level, it was confirmed that a hypoxia environment occurred inside the matrigel plug (FIG. 9). At the same time, the microvasculature in the plug was evaluated by CD31 immuno-staining. The expression of SerRS^{WT}, like SerRS^{S101D/S241D}, did not suppress microvascular formation (FIGS. 4B and 4C), suggesting that the anti-angiogenic activity of SerRS^{WT} was inactivated under hypoxia. However, as shown in FIGS. 4B and 4C, the expression of the phosphorylation-deficient SerRS^{S101A/S241A} strongly suppressed the microvessel formation in the matrigel plug, demonstrating that SerRS phosphorylation/inactivation is important for hypoxia-induced angiogenesis in vivo.

Because hypoxia-induced angiogenesis is critical for solid tumor growth, experiments were performed to determine if hypoxia-induced SerRS phosphorylation/inactivation is important for tumor angiogenesis. Human breast cancer cells MDA-MB-231 were stably transfected with human SerRS^{WT}, SerRS^{S101A/S241A}, or SerRS^{S101D/S241D} gene to give high levels of overexpression (˜10-fold) compare to that of the endogenous protein (FIG. 4D). The engineered cells were implanted subcutaneously into the mammal glands of immune deficient NOD scid gamma (NSG) mice. Two weeks later, the vasculature in the tumor xenografts were examined by CD31 staining (FIGS. 4E and 4F). In this system, SerRS^{WT} suppressed tumor angiogenesis, possibly because the high expression level of SerRS saturated the phosphorylation capacity of ATM/ATR. Nevertheless, SerRS^{S101A/S241A} exhibited a much stronger inhibition of angiogenesis compared to SerRS^{WT} (FIGS. 4E and 4F). Interestingly, SerRS^{S101D/S241D} had a strong activity in promoting tumor angiogenesis. Possibly, overexpression of SerRS^{S101D/S241D} has sequestered SIRT2, which is known to have an anti-angiogenic function (Shi et al., 2014). These results demonstrated the critical role of phosphorylation of SerRS in hypoxia-induced tumor angiogenesis

### Example 9

### Identification of Phosphorylation Sites in SerRS

Human SerRS protein was studied for post translational modification. The gel band corresponding to the size of SerRS was destained, reduced (10 mM DTT), alkylated (55 mM idoacetamide) and digested with trypsin overnight before being analyzed by nano-LC-MS/MS. The raw data was searched against the custom sequence database which contains the sequence provided and the protein of interest was identified with 31 unique peptides and 62% sequence coverage. The MS/MS data was searched against the given sequence for possible phosphorylation on serine. Phosphorylation sites were found on S79, S86, S394, and S396 of the human SerRS protein.

### Example 10

### Effect on VEGFA Expression by Modification on Phosphorylation Sites on SerRS

In this example, wildtype human SerRS protein and a number of mutant human SerRS proteins were studied for their ability to affect VEGFA expression.

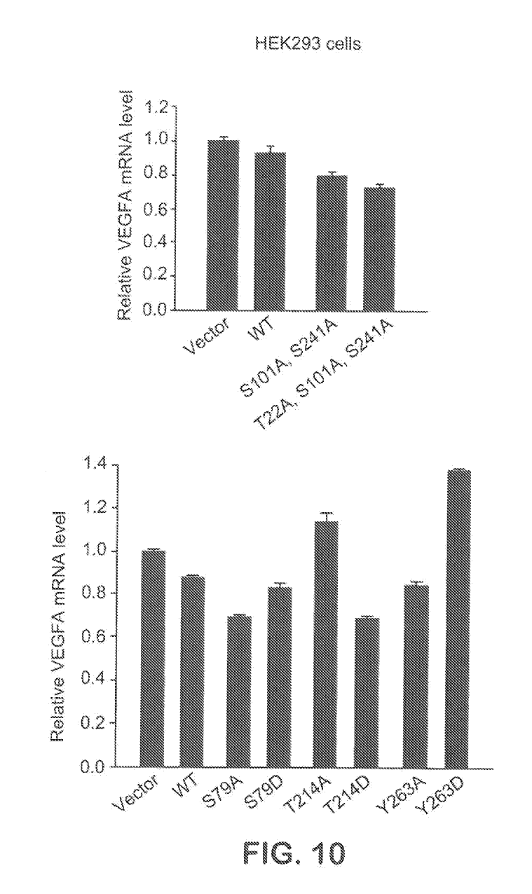

HEK293 cells were transfected with wild type (WT) SerRS or SerRS mutants. Potential phosphorylatable residues (Serine (S), Threonine (T) or Tyrosine (Y)) were substituted with alanine (A) or aspartic acid (D) to mimic unphosphorylated and phosphorylated state, respectively. At 24 hours post-transfection, cells were harvested and VEGFA expression levels were measured by qRT-PCR and relative VEGFA transcription were plotted after being normalized to 1-actin (means±: SEM). The results are shown in FIG. 10. As shown in FIG. 10, modification of phosphorylation sites on SerRS can alter SerRS' ability to regulate VEGFA expression.

### Example 11

### Binding of Endogenous SerRS on VEGFA Promoter During Hypoxia

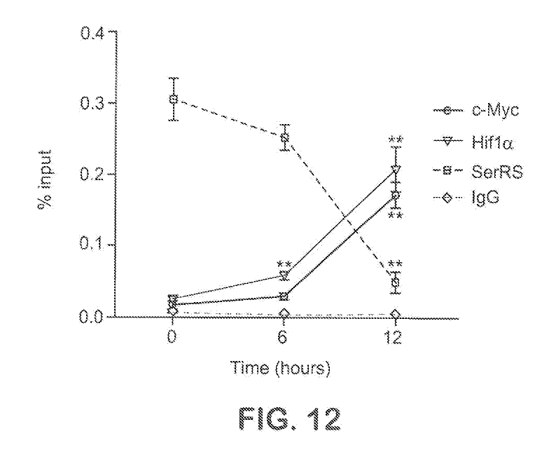

The effect of hypoxia on the binding of SerRS, c-Myc, and Hif1α to the VEGFA promoter in HEK293 cells was examined by chromatin IP (ChIP). As shown in FIG. 12, DNA binding of SerRS was reduced during hypoxia, which was concurrent with the increase of DNA binding of c-Myc and Hif1α.

The decrease in DNA binding of SerRS is thought to be caused by SerRS phosphorylation during hypoxia. The concurrent increase in DNA binding of Hif1α and c-Myc indicates that inactivation of SerRS may be required for the activation of both Myc and Hif1α.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least, one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of reducing tumor progression in a subject, comprising:
administering a composition comprising a mutant seryl-tRNA synthetase (SerRS) protein to a subject in need, wherein the mutant SerRS protein is a phosphorylation-deficient mutant SerRS protein,
whereby tumor progression is reduced in the subject.

2. (canceled)

3. The method of claim 1, wherein the mutant SerRS protein has a decreased level of phosphorylation by ataxia telangiectasia mutated kinase (ATM), ataxia telangiectasia and Rad3-related kinase (ATR), or both.

4. (canceled)

5. (canceled)

6. The method of claim 1, wherein the mutant SerRS protein comprises an amino acid substitution or an amino acid deletion at one or more of residues T22, X79, S86, X101, X142, S217, S241, S255, S258, S262, S368, S394, S396, T214, T501, X220, Y248, and Y263 relative to the corresponding wildtype SerRS protein, wherein X is serine, tyrosine or threonine.

7. (canceled)

8. The method of claim 6, wherein the mutant SerRS protein comprises an amino acid substitution X101A, S241A, or both relative to the corresponding wildtype SerRS protein, wherein X is serine or threonine.

9. (canceled)

10. (canceled)

11. The method of claim 1, wherein the mutant SerRS protein is a vertebrate SerRS protein.

12. (canceled)

13. The method of claim 1, wherein the mutant SerRS protein comprises are amino add sequence having at least 90% identity to the amino add sequence set forth in SEQ ID NO:1, SEQ ID NO: 42, SEQ ID NO: 44 or SEQ ID NO: 46, and comprise an amino acid deletion at one or both of residues X101 and S241 of SEQ ID NO: 1, SEQ ID NO: 42, SEQ ID NO: 44, or SEQ ID NO: 46, wherein X is serine or threonine.

14. The method of claim 1, wherein the mutant SerRS protein comprises an amino acid sequence having at least 90% identity to the amino add sequence set forth in SEQ ID NO: 1 and comprises an amino add substitution at one or both of residues S101 and S241 of SEQ ID NO: 1 wherein the amino acid substitution is selected from the group consisting of serine-to-arginine, serine-to-glycine, serine-to-lysine, serine-to-arginine, serine-to-asparagine, serine-to-glutamine, serine-to-histidine, serine-to-cysteine, serine-to-valine, serine-to-leucine, serine-to-isoleucine, serine-to-proline, serine-to-methionine, serine-to-tryptophan, and serine-to-phenylalanine.

15. (canceled)

16. The method of claim 1, wherein the mutant SerRS protein comprises an amino add sequence set forth in SEQ ID NO: 2, SEQ ID NO: 3, or SEQ ID NO:4.

17. The method of claim 1, wherein the reduction of tumor progression is achieved by reducing angiogenesis in the subject.

18. (canceled)

19. (canceled)

20. The method of claim 1, the tumor is solid tumor, or hematological malignancy.

21. (canceled)

22. (canceled)

23. (canceled)

24. The method of claim 1, wherein the phosphorylation-deficient mutant SerRS protein represses transcription of vascular endothelial growth factor (VEGF) in the subject.

25. (canceled)

26. The method of claim 1, wherein the tumor progression in the subject is reduced by at least 50% as compared to subjects received no treatment.

27. A mutant seryl-tRNA synthetase (SerRS) protein, wherein the mutant SerRS protein is phosphorylation-deficient, or is deficiency in repressing VEGF transcription as compared to the corresponding wildtype SerRS protein, or is effective in stimulating VEGF transcription, wherein the mutant SerRS protein comprises an amino acid substitution or an amino acid deletion at one or more of residues T22, X79, S86, X101, X142, S217, S241, S255, S258, S262, S368, S394, S396, T214, T501, X220, Y248, and Y263 relative to the corresponding wildtype SerRS protein, wherein X is serine, tyrosine or threonine.

28. (canceled)

29. The mutant SerRS protein of claim 27, wherein the mutant SerRS protein comprises an amino acid substitution at X101, S241, or both relative to the corresponding wildtype SerRS protein, wherein X is serine or threonine.

30. The mutant SerRS protein of claim 29, wherein the mutant SerRS protein comprises one of more of amino acid substitutions X101A, S241A, X101D, S241D relative to the corresponding wildtype SerRS protein, wherein X is serine or threonine.

31. (canceled)

32. (canceled)

33. The mutant SerRS protein of claim 27, wherein the mutant SerRS protein is a vertebrate protein.

34. (canceled)

35. The mutant SerRS protein of claim 27, wherein the mutant SerRS protein comprises an amino acid sequence having at least 90% identity to the amino add sequence set forth in SEQ ID NO: 1, SEQ ID NO: 42, SEQ ID NO: 44, or SEQ ID NO: 46, and comprises an amino acid deletion at one or both of residues X101 and S241, wherein X is serine or threonine.

36. The mutant SerRS protein of claim 27, wherein the mutant SerRS protein comprises an amino add sequence having at least 90% identity to the amino acid sequence set forth in SEQ ID NO: 1 and comprises an amino acid substitution at one or both of residue S101 and S241 in SEQ ID NO: 1, wherein the amino acid substitution is selected from serine-to-alanine, serine-to-glycine, serine-to-lysine, serine-to-arginine, serine-to-asparagine, serine-to-glutamine, serine-to-histidine, serine-to-cysteine, serine-to-valine, serine-to-leucine, serine-to-isoleucine, serine-to-proline, serine-to-methionine, serine-to-tryptophan, and serine-to-phenylalanine.

37. (canceled)

38. The mutant SerRS protein of claim 27, wherein the mutant SerRS protein comprises an amino acid sequence set forth in SEQ ID NO: 2, SEQ ID NO: 3, or SEQ ID NO: 4, wherein the mutant SerRS protein is deficiency in repressing VEGF transcription as compared to the corresponding wildtype SerRS protein, or is effective in stimulating VEGF transcription.

39.-67. (canceled)


SEQUENCE LISTING (46 sequences)

SEQ ID NO: 1 - protein, 514 aa, Homo Sapiens
MVLDLDLFRVDKGGDPALIRETQEKRFKDPGLVDQLVKADSEWRRCRFRADNLNKLKNLCSKTIGEKMKKKEPVGDDESVPENVLSFDDLTADALANLKVSQIKKVRLLIDEAILKCDAERIKLEAERFENLREIGNLLHPSVPISNDEDVDNKVERIWGDCTVRKKYSHVDLVVMVDGFEGEKGAVVAGSRGYFLKGVLVFLEQALIQYALRTLGSRGYIPIYTPFFMRKEVMQEVAQLSQFDEELYKVIGKGSEKSDDNSYDEKYLIATSEQPIAALHRDEWLRPEDLPIKYAGLSTCFRQEVGSHGRDTRGIFRVHQFEKIEQFVYSSPHDNKSWEMFEEMITTAEEFYQSLGIPYHIVNIVSGSLNHAASKKLDLEAWFPGSGAFRELVSCSNCTDYQARRLRIRYGQTKKMMDKVEFVHMLNATMCATTRTICAILENYQTEKGITVPEKLKEFMPPGLQELIPFVKPAPIEQEPSKKQKKQHEGSKKKAAARDVTLENRLQNMEVTDA

SEQ ID NO: 2 - protein, 514 aa, Homo sapiens
MVLDLDLFRVDKGGDPALIRETQEKRFKDPGLVDQLVKADSEWRRCRFRADNLNKLKNLCSKTIGEKMKKKEPVGDDESVPENVLSFDDLTADALANLKVAQIKKVRLLIDEAILKCDAERIKLEAERFENLREIGNLLHPSVPISNDEDVDNKVERIWGDCTVRKKYSHVDLVVMVDGFEGEKGAVVAGSRGYFLKGVLVFLEQALIQYALRTLGSRGYIPIYTPFFMRKEVMQEVAQLSQFDEELYKVIGKGSEKSDDNSYDEKYLIATSEQPIAALHRDEWLRPEDLPIKYAGLSTCFRQEVGSHGRDTRGIFRVHQFEKIEQFVYSSPHDNKSWEMFEEMITTAEEFYQSLGIPYHIVNIVSGSLNHAASKKLDLEAWFPGSGAFRELVSCSNCTDYQARRLRIRYGQTKKMMDKVEFVHMLNATMCATTRTICAILENYQTEKGITVPEKLKEFMPPGLQELIPFVKPAPIEQEPSKKQKKQHEGSKKKAAARDVTLENRLQNMEVTDA

SEQ ID NO: 3 - protein, 514 aa, Homo sapiens
MVLDLDLFRVDKGGDPALIRETQEKRFKDPGLVDQLVKADSEWRRCRFRADNLNKLKNLCSKTIGEKMKKKEPVGDDESVPENVLSFDDLTADALANLKVSQIKKVRLLIDEAILKCDAERIKLEAERFENLREIGNLLHPSVPISNDEDVDNKVERIWGDCTVRKKYSHVDLVVMVDGFEGEKGAVVAGSRGYFLKGVLVFLEQALIQYALRTLGSRGYIPIYTPFFMRKEVMQEVAQLAQFDEELYKVIGKGSEKSDDNSYDEKYLIATSEQPIAALHRDEWLRPEDLPIKYAGLSTCFRQEVGSHGRDTRGIFRVHQFEKIEQFVYSSPHDNKSWEMFEEMITTAEEFYQSLGIPYHIVNIVSGSLNHAASKKLDLEAWFPGSGAFRELVSCSNCTDYQARRLRIRYGQTKKMMDKVEFVHMLNATMCATTRTICAILENYQTEKGITVPEKLKEFMPPGLQELIPFVKPAPIEQEPSKKQKKQHEGSKKKAAARDVTLENRLQNMEVTDA

SEQ ID NO: 4 - protein, 514 aa, Homo sapiens
MVLDLDLFRVDKGGDPALIRETQEKRFKDPGLVDQLVKADSEWRRCRFRADNLNKLKNLCSKTIGEKMKKKEPVGDDESVPENVLSFDDLTADALANLKVAQIKKVRLLIDEAILKCDAERIKLEAERFENLREIGNLLHPSVPISNDEDVDNKVERIWGDCTVRKKYSHVDLVVMVDGFEGEKGAVVAGSRGYFLKGVLVFLEQALIQYALRTLGSRGYIPIYTPFFMRKEVMQEVAQLAQFDEELYKVIGKGSEKSDDNSYDEKYLIATSEQPIAALHRDEWLRPEDLPIKYAGLSTCFRQEVGSHGRDTRGIFRVHQFEKIEQFVYSSPHDNKSWEMFEEMITTAEEFYQSLGIPYHIVNIVSGSLNHAASKKLDLEAWFPGSGAFRELVSCSNCTDYQARRLRIRYGQTKKMMDKVEFVHMLNATMCATTRTICAILENYQTEKGITVPEKLKEFMPPGLQELIPFVKPAPIEQEPSKKQKKQHEGSKKKAAARDVTLENRLQNMEVTDA

SEQ ID NO: 5 - protein, 514 aa, Homo sapiens
MVLDLDLFRVDKGGDPALIRETQEKRFKDPGLVDQLVKADSEWRRCRFRADNLNKLKNLCSKTIGEKMKKKEPVGDDESVPENVLSFDDLTADALANLKVDQIKKVRLLIDEAILKCDAERIKLEAERFENLREIGNLLHPSVPISNDEDVDNKVERIWGDCTVRKKYSHVDLVVMVDGFEGEKGAVVAGSRGYFLKGVLVFLEQALIQYALRTLGSRGYIPIYTPFFMRKEVMQEVAQLDQFDEELYKVIGKGSEKSDDNSYDEKYLIATSEQPIAALHRDEWLRPEDLPIKYAGLSTCFRQEVGSHGRDTRGIFRVHQFEKIEQFVYSSPHDNKSWEMFEEMITTAEEFYQSLGIPYHIVNIVSGSLNHAASKKLDLEAWFPGSGAFRELVSCSNCTDYQARRLRIRYGQTKKMMDKVEFVHMLNATMCATTRTICAILENYQTEKGITVPEKLKEFMPPGLQELIPFVKPAPIEQEPSKKQKKQHEGSKKKAAARDVTLENRLQNMEVTDA

SEQ ID NO: 6 - protein, 514 aa, Homo sapiens
MVLDLDLFRVDKGGDPALIRETQEKRFKDPGLVDQLVKADSEWRRCRFRADNLNKLKNLCSKTIGEKMKKKEPVGDDESVPENVLSFDDLTADALANLKVEQIKKVRLLIDEAILKCDAERIKLEAERFENLREIGNLLHPSVPISNDEDVDNKVERIWGDCTVRKKYSHVDLVVMVDGFEGEKGAVVAGSRGYFLKGVLVFLEQALIQYALRTLGSRGYIPIYTPFFMRKEVMQEVAQLEQFDEELYKVIGKGSEKSDDNSYDEKYLIATSEQPIAALHRDEWLRPEDLPIKYAGLSTCFRQEVGSHGRDTRGIFRVHQFEKIEQFVYSSPHDNKSWEMFEEMITTAEEFYQSLGIPYHIVNIVSGSLNHAASKKLDLEAWFPGSGAFRELVSCSNCTDYQARRLRIRYGQTKKMMDKVEFVHMLNATMCATTRTICAILENYQTEKGITVPEKLKEFMPPGLQELIPFVKPAPIEQEPSKKQKKQHEGSKKKAAARDVTLENRLQNMEVTDA

SEQ ID NO: 7 - DNA, 38 bp, Artificial Sequence
  Synthetic oligonucleotide
gaaagtcgcacaaatcaaaaaagtccgactcctcattg

SEQ ID NO: 8 - DNA, 34 bp, Artificial Sequence
  Synthetic oligonucleotide
tgatttgtgcgactttcaggttagctaaagcgtc

SEQ ID NO: 9 - DNA, 38 bp, Artificial Sequence
  Synthetic oligonucleotide
gaaagtcgaccaaatcaaaaaagtccgactcctcattg

SEQ ID NO: 10 - DNA, 34 bp, Artificial Sequence
  Synthetic oligonucleotide
tgatttggtcgactttcaggttagctaaagcgtc

SEQ ID NO: 11 - DNA, 33 bp, Artificial Sequence
  Synthetic oligonucleotide
agctcgcacagtttgatgaagaactttataagg

SEQ ID NO: 12 - DNA, 36 bp, Artificial Sequence
  Synthetic oligonucleotide
aactgtgcgagctgtgccacctcctgcatgacctcc

SEQ ID NO: 13 - DNA, 33 bp, Artificial Sequence
  Synthetic oligonucleotide
agctcgaccagtttgatgaagaactttataagg

SEQ ID NO: 14 - DNA, 36 bp, Artificial Sequence
  Synthetic oligonucleotide
aactggtcgagctgtgccacctcctgcatgacctcc

SEQ ID NO: 15 - DNA, 38 bp, Artificial Sequence
  Synthetic oligonucleotide
gaaagtcgcacagattaaaaaagtccgactcctcattg

SEQ ID NO: 16 - DNA, 34 bp, Artificial Sequence
  Synthetic oligonucleotide
taatctgtgcgactttcagggcagctagcgcgtc

SEQ ID NO: 17 - DNA, 38 bp, Artificial Sequence
  Synthetic oligonucleotide
gaaagtcgaccagattaaaaaagtccgactcctcattg

SEQ ID NO: 18 - DNA, 34 bp, Artificial Sequence
  Synthetic oligonucleotide
taatctggtcgactttcagggcagctagcgcgtc

SEQ ID NO: 19 - DNA, 36 bp, Artificial Sequence
  Synthetic oligonucleotide
cagctcgcccagtttgatgaagaactttataaggtg

SEQ ID NO: 20 - DNA, 32 bp, Artificial Sequence
  Synthetic oligonucleotide
caaactgggcgagctgggccacttcctgcatg

SEQ ID NO: 21 - DNA, 36 bp, Artificial Sequence
  Synthetic oligonucleotide
cagctcgaccagtttgatgaagaactttataaggtg

SEQ ID NO: 22 - DNA, 32 bp, Artificial Sequence
  Synthetic oligonucleotide
caaactggtcgagctgggccacttcctgcatg

SEQ ID NO: 23 - DNA, 21 bp, Artificial Sequence
  Synthetic oligonucleotide
ggcatagggacccatcattga

SEQ ID NO: 24 - DNA, 21 bp, Artificial Sequence
  Synthetic oligonucleotide
gcatggagtatctcacaaagt

SEQ ID NO: 25 - DNA, 19 bp, Artificial Sequence
  Synthetic oligonucleotide
taaggctatgaagagatac

SEQ ID NO: 26 - DNA, 21 bp, Artificial Sequence
  Synthetic oligonucleotide
gagggcagaatcatcacgaag

SEQ ID NO: 27 - DNA, 24 bp, Artificial Sequence
  Synthetic oligonucleotide
tgtgctgtaggaagctcatctctc

SEQ ID NO: 28 - DNA, 18 bp, Artificial Sequence
  Synthetic oligonucleotide
cgtcaccaactgggacga

SEQ ID NO: 29 - DNA, 17 bp, Artificial Sequence
  Synthetic oligonucleotide
atgggggagggcatacc

SEQ ID NO: 30 - DNA, 21 bp, Artificial Sequence
  Synthetic oligonucleotide
ggctctcctccatctgtctgc

SEQ ID NO: 31 - DNA, 24 bp, Artificial Sequence
  Synthetic oligonucleotide
cagtggttttctttctttgctttg

SEQ ID NO: 32 - DNA, 21 bp, Artificial Sequence
  Synthetic oligonucleotide
tcaccaccacagccgaaagag

SEQ ID NO: 33 - DNA, 21 bp, Artificial Sequence
  Synthetic oligonucleotide
gtcagcaatgccagggtacat

SEQ ID NO: 34 - DNA, 25 bp, Artificial Sequence
  Synthetic oligonucleotide
aggagaatgtgaacaaacctgacac

SEQ ID NO: 35 - DNA, 25 bp, Artificial Sequence
  Synthetic oligonucleotide
cctcttacctcagttacaatttata

SEQ ID NO: 36 - DNA, 27 bp, Artificial Sequence
  Synthetic oligonucleotide
ggcggggcggagccatgcgcccccccc

SEQ ID NO: 37 - DNA, 20 bp, Artificial Sequence
  Synthetic oligonucleotide
gggcggatgggtaattttca

SEQ ID NO: 38 - DNA, 18 bp, Artificial Sequence
  Synthetic oligonucleotide
ctgcggacgcccagtgaa

SEQ ID NO: 39 - DNA, 1545 bp, Homo sapiens
atggtgctggatctggatttgtttcgggtggataaaggaggggacccagccctcatccgagagacgcaggagaagcgcttcaaggacccgggactagtggaccagctggtgaaggcagacagcgagtggcgacgatgtagatttcgggcagacaacttgaacaagctgaagaacctatgcagcaagacaatcggagagaaaatgaagaaaaaagagccagtgggagatgatgagtctgtcccagagaatgtgctgagtttcgatgaccttactgcagacgctttagctaacctgaaagtctcacaaatcaaaaaagtccgactcctcattgatgaagccatcctgaagtgtgacgcggagcggataaagttggaagcagagcggtttgagaacctccgagagattgggaaccttctgcacccttctgtacccatcagtaacgatgaggatgtggacaacaaagtagagaggatttggggtgattgtacagtcaggaagaagtactctcatgtggacctggtggtgatggtagatggctttgaaggcgaaaagggggccgtggtggctgggagtcgagggtacttcttgaagggggtcctggtgttcctggaacaggctctcatccagtatgcccttcgcaccttgggaagtcggggctacattcccatttataccccctttttcatgaggaaggaggtcatgcaggaggtggcacagctcagccagtttgatgaagaactttataaggtgattggcaaaggcagtgaaaagtctgatgacaactcctatgatgagaagtacctgattgccacctcagagcagcccattgctgccctgcaccgggatgagtggctccggccggaggacctgcccatcaagtatgctggcctgtctacctgcttccgtcaggaggtgggctcccatggccgtgacacccgtggcatcttccgagtccatcagtttgagaagattgaacagtttgtgtactcatcaccccatgacaacaagtcatgggagatgtttgaagagatgattaccaccgcagaggagttctaccagtccctggggattccttaccacattgtgaatattgtctcaggttctttgaatcatgctgccagtaagaagcttgacctggaggcctggtttccgggctcaggagccttccgtgagttggtctcctgttctaattgcacggattaccaggctcgccggcttcgaatccgatatgggcaaaccaagaagatgatggacaaggtggagtttgtccatatgctcaatgctaccatgtgcgccactacccgtaccatctgcgccatcctggagaactaccagacagagaagggcatcactgtgcctgagaaattgaaggagttcatgccgccaggactgcaagaactgatcccctttgtgaagcctgcgcccattgagcaggagccatcaaagaagcagaagaagcaacatgagggcagcaaaaagaaagcagcagcaagagacgtcaccctagaaaacaggctgcagaacatggaggtcaccgatgcttga

SEQ ID NO: 40 - DNA, 1545 bp, Homo sapiens
atggtgctggatctggatttgtttcgggtggataaaggaggggacccagccctcatccgagagacgcaggagaagcgcttcaaggacccgggactagtggaccagctggtgaaggcagacagcgagtggcgacgatgtagatttcgggcagacaacttgaacaagctgaagaacctatgcagcaagacaatcggagagaaaatgaagaaaaaagagccagtgggagatgatgagtctgtcccagagaatgtgctgagtttcgatgaccttactgcagacgctttagctaacctgaaagtcgcvcaaatcaaaaaagtccgactcctcattgatgaagccatcctgaagtgtgacgcggagcggataaagttggaagcagagcggtttgagaacctccgagagattgggaaccttctgcacccttctgtacccatcagtaacgatgaggatgtggacaacaaagtagagaggatttggggtgattgtacagtcaggaagaagtactctcatgtggacctggtggtgatggtagatggctttgaaggcgaaaagggggccgtggtggctgggagtcgagggtacttcttgaagggggtcctggtgttcctggaacaggctctcatccagtatgcccttcgcaccttgggaagtcggggctacattcccatttataccccctttttcatgaggaaggaggtcatgcaggaggtggcacagctcgcvcagtttgatgaagaactttataaggtgattggcaaaggcagtgaaaagtctgatgacaactcctatgatgagaagtacctgattgccacctcagagcagcccattgctgccctgcaccgggatgagtggctccggccggaggacctgcccatcaagtatgctggcctgtctacctgcttccgtcaggaggtgggctcccatggccgtgacacccgtggcatcttccgagtccatcagtttgagaagattgaacagtttgtgtactcatcaccccatgacaacaagtcatgggagatgtttgaagagatgattaccaccgcagaggagttctaccagtccctggggattccttaccacattgtgaatattgtctcaggttctttgaatcatgctgccagtaagaagcttgacctggaggcctggtttccgggctcaggagccttccgtgagttggtctcctgttctaattgcacggattaccaggctcgccggcttcgaatccgatatgggcaaaccaagaagatgatggacaaggtggagtttgtccatatgctcaatgctaccatgtgcgccactacccgtaccatctgcgccatcctggagaactaccagacagagaagggcatcactgtgcctgagaaattgaaggagttcatgccgccaggactgcaagaactgatcccctttgtgaagcctgcgcccattgagcaggagccatcaaagaagcagaagaagcaacatgagggcagcaaaaagaaagcagcagcaagagacgtcaccctagaaaacaggctgcagaacatggaggtcaccgatgcttga

SEQ ID NO: 41 - DNA, 1539 bp, Mus musculus
atggtgctggacctggatttgtttcgggtggataaaggaggggacccagccctcattcgagagacgcaggagaagcgcttcaaggacccggggctggtggaccagctggtgaaagcagacagtgagtggcgacgatgcagatttcgggcagacaacttgaacaagctgaagaatttatgcagcaaaactattggggagaaaatgaagaaaaaggaagcagtgggagacgacgagtccgtcccagagaacgtgctgaatttcgatgacctcactgcagacgcgctagctgccctgaaagtctcacagattaaaaaagtccgactcctcattgatgaagccatccagaagtgtgatggggagcgggtaaagctggaagcagagcgatttgagaacctccgcgagattgggaaccttctgcacccctctgtgcccattagtaatgatgaggacgcagacaacaaagtagaacgtatttggggagattgtacagtcaggaagaagtattcccatgtggacctggtggtgatggtagatggctttgaaggcgaaaagggagccgtggtggctggtagtcgggggtacttcctgaaggggcccctggtgttcctggagcaggcgcttatccaatatgcactgcgtaccttggggagtcggggctacactccaatctacacccccttcttcatgaggaaagaggtcatgcaggaagtggcccagctcagccagtttgatgaagaactttataaggtgattggcaaaggcagcgaaaagtcagatgacaactcctatgacgagaaatacttgattgccacctcagagcagcccatcgcggctctgcaccgggacgagtggctgcggccagaggatctgcccatcaagtacgctggcctctccacctgctttcgtcaggaagtgggctcgcatggccgtgacacccgtggtatcttccgagtccatcagtttgagaagattgagcagtttgtgtactcatcgccccatgacaataagtcgtgggagatgtttgatgagatgatcgccaccgcagaagaattctaccagtctttggggatcccttaccacattgtgaatattgtctcaggctccttgaatcacgctgccagtaagaagctcgacctggaggcctggttcccaggctcgggtgccttccgtgagttggtgtcctgttctaattgcacggattaccaagctcgccgcctgagaatccgatatgggcagaccaagaagatgatggacaaggtggagtttgtccatatgcttaatgctacaatgtgtgctaccacccggaccatctgcgccatcctggagaactaccaggcagagaagggcatcgctgtgccagagaagttgagggagttcatgccgccagggctccaagagctgatcccgtttgtgaagcctgcacccattgaccaggagccatctaagaagcagaagaagcaacatgaaggcagcaaaaagaaagcgaaagaggtccccctggagaaccagctgcagagcatggaggtcactgaggcctga

SEQ ID NO: 42 - protein, 512 aa, Mus musculus
MVLDLDLFRVDKGGDPALIRETQEKRFKDPGLVDQLVKADSEWRRCRFRADNLNKLKNLCSKTIGEKMKKKEAVGDDESVPENVLNFDDLTADALAALKVSQIKKVRLLIDEAIQKCDGERVKLEAERFENLREIGNLLHPSVPISNDEDADNKVERIWGDCTVRKKYSHVDLVVMVDGFEGEKGAVVAGSRGYFLKGPLVFLEQALIQYALRTLGSRGYTPIYTPFFMRKEVMQEVAQLSQFDEELYKVIGKGSEKSDDNSYDEKYLIATSEQPIAALHRDEWLRPEDLPIKYAGLSTCFRQEVGSHGRDTRGIFRVHQFEKIEQFVYSSPHDNKSWEMFDEMIATAEEFYQSLGIPYHIVNIVSGSLNHAASKKLDLEAWFPGSGAFRELVSCSNCTDYQARRLRIRYGQTKKMMDKVEFVHMLNATMCATTRTICAILENYQAEKGIAVPEKLREFMPPGLQELIPFVKPAPIDQEPSKKQKKQHEGSKKKAKEVPLENQLQSMEVTEA

SEQ ID NO: 43 - DNA, 1548 bp, Danio rerio
atggtgctcgatttagacctgtttcgcaccgacaaaggcggcgatcctgaaattatccgggaaactcagaggaaacggttcaaagatgtgtctctggtggataaactggtccaggcggacacagaatggagaaaatgtcgtttcacagcagataaccttaacaaggccaagaatctctgcagcaaatccatcggtgaaaagatgaagaagaaagagccagtaggggatgatgacactcttccagaagaggctcagaatctggaagccctcactgcagaaacgttatcgccgcttactgtgactcagataaagaaagtgcgggttctggtggatgaggctgtgcagaagacagacagtgaccggctgaagctggaggcagagcgctttgagtatctgcgagagatcggcaacctcctacatccctctgtgcccatcagcaacgatgaggatgctgataataaagtggagcgcacctggggtgactgcacggtgcagaagaagtactctcatgtggacctggtcgtcatggttgatggatatgagggggaaaaaggagccattgttgctggaagcagaggatactttctcaaggggcctttagtgttcttggagcaagctttgattaactatgcgctgcggatcctgtacagcaagaactacaacctcctgtacacacccttcttcatgaggaaagaagtcatgcaggaggtcgctcagctcagccagtttgacgaggagctctacaaggtgatcgggaaaggaagtgagaagtctgatgataacacagtggacgagaagtacttgattgccacatcagagcagccaatcgcagccttcctgagagatgagtggctgaagccagaagaacttcctatccgctacgctggcctctccacctgcttcagacaggaagtgggctctcatggcagagacacgcgcgggatcttcagggtccatcagtttgagaagattgagcagtttgtgtacgcctctcctcatgatggcaaatcctgggagatgtttgatgaaatgattggaaccgctgaatccttttatcaaacattaggaattccttatcgaattgtcaacatcgtgtcaggtgctttgaaccacgcagctagtaaaaagctggatttagaggcttggtttcctggttcccaggcttttagagagcttgtgtcatgctcaaactgtacagactatcaggctcgtcgcttgcggattcgatacgggcaaactaagaaaatgatggacaaggctgagtttgtgcacatgctcaatgccaccatgtgtgcgaccactcgtgtcatctgtgccatcctggagaacttccaaacagaggaaggcatcattgttccagaacccctcaaggcattcatgcctccaggtttaacagaaataattaagtttgtgaagccagcccccattgaccaggaaaccacaaagaagcagaagaaacagcaggaaggaggaaagaagaagaaacatcagggcggcgatgctgatctagagaacaaagtggagaacatgtctgtcaatgactcttag

SEQ ID NO: 44 - protein, 515 aa, Danio rerio
MVLDLDLFRTDKGGDPEIIRETQRKRFKDVSLVDKLVQADTEWRKCRFTADNLNKAKNLCSKSIGEKMKKKEPVGDDDTLPEEAQNLEALTAETLSPLTVTQIKKVRVLVDEAVQKTDSDRLKLEAERFEYLREIGNLLHPSVPISNDEDADNKVERTWGDCTVQKKYSHVDLVVMVDGYEGEKGAIVAGSRGYFLKGPLVFLEQALINYALRILYSKNYNLLYTPFFMRKEVMQEVAQLSQFDEELYKVIGKGSEKSDDNTVDEKYLIATSEQPIAAFLRDEWLKPEELPIRYAGLSTCFRQEVGSHGRDTRGIFRVHQFEKIEQFVYASPHDGKSWEMFDEMIGTAESFYQTLGIPYRIVNIVSGALNHAASKKLDLEAWFPGSQAFRELVSCSNCTDYQARRLRIRYGQTKKMMDKAEFVHMLNATMCATTRVICAILENFQTEEGIIVPEPLKAFMPPGLTEIIKFVKPAPIDQETTKKQKKQQEGGKKKKHQGGDADLENKVENMSVNDS

SEQ ID NO: 45 - DNA, 1536 bp, Xenopus tropicalis
atggttctagatttggatcttttccgggaggacaagggaggaaacccggagctcatcagagagactcagagaaagagatttaaggacccggggctggtggatgcattgctgaactcagacacggcctggagaaagtgcaggtttcaggcagacaatcttaataaacagaaaaatctttgcagcaaaatcatcggggagaaaatgaagaaaaaggagccgttgggagacagtgatgttcttcctgaaaatatccagcttgaccagctaactgctgaagttcttagtgctctgtcagttacacagataaaaagactccgggtcttaatagatgaagccatagcagcaactgacacagaacgtatcaagctggaggctgagaggtttgaaagtttacgtgagattggaaacctgcttcacccaacagtgcctatcagtaacgatgaggacaatgataataaggtggaacgcacttggggagactgtgaagttcgaaagagatactcacatgtggaccttgtggttatggtggatggctttgagggggaaaaaggagctgtagtagctggtagcagaggatatttcttaaagggtcctctggtgtttctagagcaggccctcatacagtttgctttgcataccctggcagaaaagcaatacacccccatatataccccatttttcatgagaaaagaggtcatgcaggaggtggctcagctcagtcaatttgatgaagaactgtacaaggtgataggtaaaggtagtgagaaatctgatgataactccatagatgagaagtacctaatagccacttcagaacagccaatagctgcattacatcgggatgagtggctgaagcctgaagagttgcctctacgatatgctggcatatcaacttgtttccgtcaggaagtgggctcccatggaagagacacaagaggcatatttagagtacaccagtttgagaagattgagcagtttatttatgcctcacctaatgataacaagtcctgggaattgtttgaagaaatgattatgacagctgaatcattttaccagaagcttggcattccctatcgtattgtgaatattgtttcaggctccttgaaccacgctgccagtaaaaagctggatttagaggcctggtttcctggctcaggtgcattcagagagctggtctcttgttcaaactgcactgactaccaagcccggcggctacggatccgatatgggcagacaaagaagatgatggacaaggtagagtttgtacacatgttaaatgccaccatgtgtgccacgacccgtgcaatttgtgcaattctagagaactatcaaaccgaggaagggataattataccagagaaactcagggactttatgcctccaggtctgaattatataataaagtttgtaaaaccagctccaattgaccaggaactcaccaaaaaacagaagaagcagcagcaggaaaaaggaaagaaaacagaaaattgtggtttagataatcaaatggagaacatgaaagttaattcagcttaa

SEQ ID NO: 46 - protein, 511 aa, Xenopus tropicalis
MVLDLDLFREDKGGNPELIRETQRKRFKDPGLVDALLNSDTAWRKCRFQADNLNKQKNLCSKIIGEKMKKKEPLGDSDVLPENIQLDQLTAEVLSALSVTQIKRLRVLIDEAIAATDTERIKLEAERFESLREIGNLLHPTVPISNDEDNDNKVERTWGDCEVRKRYSHVDLVVMVDGFEGEKGAVVAGSRGYFLKGPLVFLEQALIQFALHTLAEKQYTPIYTPFFMRKEVMQEVAQLSQFDEELYKVIGKGSEKSDDNSIDEKYLIATSEQPIAALHRDEWLKPEELPLRYAGISTCFRQEVGSHGRDTRGIFRVHQFEKIEQFIYASPNDNKSWELFEEMIMTAESFYQKLGIPYRIVNIVSGSLNHAASKKLDLEAWFPGSGAFRELVSCSNCTDYQARRLRIRYGQTKKMMDKVEFVHMLNATMCATTRAICAILENYQTEEGIIIPEKLRDFMPPGLNYIIKFVKPAPIDQELTKKQKKQQQEKGKKTENCGLDNQMENMKVNSA